(19) United States
(12) Patent Application Publication
(10) Pub. No.: US 2018/0078612 A1
(43) Pub. Date: Mar. 22, 2018

(54) INTRACELLULAR DELIVERY SYSTEM AND METHODS

(71) Applicants: REGENTS OF THE UNIVERSITY OF MINNESOTA, Minneapolis, MN (US); THE REGENTS OF THE UNIVERSITY OF CALIFORNIA, Oakland, CA (US)

(72) Inventors: Joseph A. Zasadzinski, Minneapolis, MN (US); Norbert O. Reich, Santa Barbara, CA (US); Gary B. Braun, La Jolla, CA (US); Demosthenes P. Morales, Santa Barbara, CA (US); Xiao Huang, Santa Barbara, CA (US)

(21) Appl. No.: 15/830,898

(22) Filed: Dec. 4, 2017

Related U.S. Application Data
(62) Division of application No. 14/727,373, filed on Jun. 1, 2015, now abandoned.
(60) Provisional application No. 62/005,588, filed on May 30, 2014.
(60) Provisional application No. 62/019,004, filed on Jun. 30, 2014.

(51) Int. Cl. A61K 38/17; C12N 15/113

(52) U.S. Cl. CPC A61K 38/1767; C12N 2310/14; A61K 47/48861; C12N 15/113

(57) ABSTRACT

This disclosure describes, in one aspect, a composition for intracellular delivery of a polypeptide. Generally, the composition includes a substrate that has a surface, a cleavable linker affixed to at least a portion of the surface, and a cargo polypeptide bound to the cleavable linker. In some embodiments, the cargo polypeptide can further include a targeting peptide. In another aspect, this disclosure describes a method for intracellular delivery of a polypeptide. Generally, the method includes administering to a subject any embodiments of the composition summarized above, and cleaving the cleavable linker, thereby releasing the cargo polypeptide from the substrate.

### CROSS-REFERENCE TO RELATED APPLICATION

This application claims priority to U.S. Provisional Patent Application Ser. No. 62/005,588, filed May 30, 2014 and U.S. Provisional Patent Application Ser. No. 62/019,004, filed Jun. 30, 2014, each of which is incorporated herein by reference.

### GOVERNMENT FUNDING

This invention was made with government support under R01 EB012637 awarded by the National Institutes of Health. The government has certain rights in the invention.

### SUMMARY

This disclosure describes, in one aspect, a composition for intracellular delivery of a polypeptide. Generally, the composition includes a substrate that has a surface, a cleavable linker affixed to at least a portion of the surface, and a cargo polypeptide bound to the cleavable linker. In some embodiments, the cargo polypeptide can further include a targeting peptide.

In some embodiments, the cleavable linker is cleaved upon exposure to near infrared radiation.

In some embodiments, the cargo polypeptide is reversibly bound to the cleavable linker.

In another aspect, this disclosure describes a method for intracellular delivery of a polypeptide. Generally, the method includes administering to a subject any embodiments of the composition summarized above, and cleaving the cleavable linker, thereby releasing the cargo polypeptide from the substrate.

In some embodiments, cleaving the cleavable linker can involve exposing the cleavable linker to near infrared radiation.

In another aspect, this disclosure describes a composition that includes a substrate, a cleavable linker affixed to at least a portion of the surface of the substrate, and a cargo polynucleotide bound to the cleavable linker.

In some embodiments, the cleavable linker can be cleaved upon exposure to near infrared radiation.

In some embodiments, the cargo polynucleotide can be reversibly bound to the cleavable linker.

In some embodiments, the composition can further include a targeting peptide affixed to the substrate.

In another aspect, this disclosure describes a method that generally includes administering to a subject any embodiment of the composition summarized above and cleaving the cleavable linker. Cleaving the cleavable linker releases the cargo polynucleotide from the substrate.

In some embodiments, cleaving the cleavable linker can involve exposing the cleavable linker to near infrared radiation.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. (a) Schematic of protein-HGN assembly. Thiol-DNA-amine (Red) is bound to the gold surface of the hollow gold nanoshell via the thiol. N—[Nα,Nα-Bis(carboxymethyl)-L-lysine]-12-mercaptododecamamide (NTA Moeity, Gray) is attached via NHS-PEG4-Maleimide to provide a flexible linkage between the hollow gold nanoshell and the NTA-Ni binding site. His-tag-GFP-RPARPAR (Orange) is non-covalently bonded to the NTA via the mutual chelation of Ni²⁺. At least two NTA-Ni-His bonds between the hollow gold nanoshell and the GFP are necessary for long-term binding. (b) Mechanism of uptake and release from hollow gold nanoshells in cells upon 800 nm near-infrared laser excitation.

FIG. 2. (a) UV-Vis spectrophotometry of GFP-HGN demonstrating plasmon resonance at 800 nm for near-infrared excitation (black) due to the hollow core structure of the hollow gold nanoshell. The resonance peak decreases after laser exposure at 800 nm for 30 seconds (red) at 4.2 W/cm² power, consistent with partial annealing of the hollow gold structure to a solid core. These power levels are appropriate for ablating cells and could couple to a therapeutic protein designed to kill cells. However, for benign protein delivery, a power of 1.5 W/cm² provides the optimal scaffold/protein release to minimize cell toxicity. GFP fluorescence at ˜510 nm is partially quenched while in close proximity to the hollow gold nanoshell (olive) relative to free GFP (lime). Femtosecond to 100 picosecond near-infrared laser pulses thermalize the thiol-Au bond, releasing the entire DNA-Malemide-NTA-Ni-His-GFP construct, allowing the His-GFP to dissociate from the NTA-Ni due to the low avidity of single NTA-Ni-His bonds. The fluorescence increases (green) after laser irradiation, consistent with GFP release from the hollow gold nanoshell. Imidazole competition with the His-tag gives 100% release of the GFP (lime). (b) Percent release of GFP from hollow gold nanoshells after laser exposure at various power densities (1 kHz repetition rate and 130 fs pulse length) and time combinations as determined by the increase in fluorescence intensity at 535 nm after excitation at 480 nm. Short time exposures at the highest powers were optimal for GFP release, with a maximum of about 60% release. Error bars represent the upper deviation from the mean of experiments performed in triplicate. (c) Percent release of GFP from hollow gold nanoshells after addition of imidazole to nanoparticles exposed to laser from (b). Decreasing percentage of GFP after laser treatment confirms almost complete dissociation of GFP from nanoparticle and possible reabsorption of GFP to bare nanoparticle surfaces indicated by the decrease in free GFP after laser release in (b).

FIG. 3. Cell-specific targeting of GFP-HGN as revealed by co-culture of PPC-1 and M21 cells. a) Green channel: Punctate fluorescence from GFP-HGN displayed within NRP-1 expressing cells after two-hour incubation. b) Red channel: M21 cells stained with CTO prior to co-culture plating. c) Merged image showing minimal internalization of the GFP-HGN within M21 cells and near-universal internalization of GFP-HGN in PPC-1 cells. Scale bar is 50 μm.

FIG. 4. PPC-1 cells incubated with GFP-HGN for two hours and then excited with variable laser power for 3 seconds, 30 seconds, or 60 seconds. Viability was measured after 72 hours. No significant loss in cell viability is observed at laser power densities of less than or equal to 1.5 W/cm² and 60 seconds of exposure (p<0.01), which provide optimal protein release. Cells not treated with GFP-HGN (White) show minimal change in cell viability at 4.24 W/cm² over 60 seconds exposure time, showing the benign nature of NIR light alone. Error bars represent the s.e.m. of triplicates.

FIG. 5. Quantification of GFP release in PPC-1 cells after exposure to pulsed near-infrared laser. (a) GFP-HGNs are observed as punctate objects concentrated within endosomes within cell boundaries after 2 hours incubation and prior to laser excitation (Initial). The first and second NIR light exposure pass increase the average GFP fluorescence per cell, consistent with thiol bond thermalization as in FIG. 3. The fluorescence pattern is brighter and diffuses throughout the cell cytoplasm, consistent with nanobubble rupture of endosomes and release of the GFP to the cytoplasm. (b) The ratio of GFP intensity before and after two rounds of laser irradiation shows regions where release occurs and the degree of dequenching. Higher ratios are represented by saturating white pixels. The second pass releases less GFP than the first pass. (c) Close up of the blue box region for the three frames in (a). Plotting the number of pixels having a particular ratio within the white box indicates that the fold-increase in intensity is greater after the first pass than the second pass. Scale bar is 50 μm.

FIG. 6. Patterned NIR light exposure of a square region within a cell culture releases GFP only where NIR light interacts with the cells (inside red box). (a) GFP-HGNs were internalized into the PPC-1 cells as in FIG. 4 and FIG. 5, and the central box shape was irradiated by the pulsed laser. (b) After laser exposure only the cells within the box increased in brightness due to the released GFP. Intensity profiles along lines 1 and 2 are plotted. (c) Line profile 1 across the cytoplasm and nucleus show three-fold increase in GFP intensity and the accumulation in nucleoli (*). (d) Line profile 2 across a cell only partly exposed to laser irradiation demonstrates subcellular resolution of the technique. The lower half of the cell is within the excitation region ( [symbol] ), while the rest of the cell resides outside (•). The fluorescence fold increase is 3±0.5 (standard deviation) for the exposed areas of cells on the edge of the perimeter.

FIG. 7. NIR light can be delivered using a conventional 2-photon optical microscope for optimal cell-selective GFP release. (a) Red polygons outline cells targeted for laser irradiation. The blue oval is a reference cell not targeted for release. Scale bar is 100 μm. (b) Only cells within the red polygons are exposed to laser and show an increase in overall fluorescence. The reference cell in the blue oval does not undergo a change in fluorescence, even though it is only approximately 5 μm away. (c) Line profile analysis across the cytoplasm and the nucleus of cell 1 show a peak ˜5 fold increase in fluorescence intensity. (d) Line profile analysis across the cytoplasm and the nucleus of cell 2, the reference cell not selected for exposure, showing no fluorescence increase after treatment of neighboring cells.

FIG. 8. Hollow gold nanoshells were determined to be ˜40 nm in diameter by a commercial Nanosight LM10HS. Transmission electron microscopy of hollow gold nanoshells in inset suggests hollow interior as determined by decreased electron density in the center of the shells.

FIG. 9. GFP fluorescence vs. concentration standard used to relate fluorescence intensity to GFP concentration in all experiments.

FIG. 10. During assembly, GFP associates with hollow gold nanoshells through the formation of non-covalent coordinate bonds with NTA-nickel (Ni²⁺). GFP bound to HGN were treated with imidazole for 10 minutes; the HGN centrifuged and pelleted, and the fluorescence of the supernatant was determined and compared to FIG. 9 to determine the amount of GFP bound to the HGN. GFP-HGN with nickel had the highest amount of associated GFP, while hollow gold nanoshells without nickel and/or without NTA showed minimal binding. Error bars are standard deviation of samples prepared in triplicate.

FIG. 11. 32 pM GFP-HGN were incubated with a suspension of 500,000 cells for two hours at 37° C. and 5% CO₂. To estimate amount of GFP incorporated into cells, GFP fluorescence was measured upon imidazole treatment of GFP-HGN extracted from PPC-1 cell pellet and compared to the GFP fluorescence of a standard curve. The black dashed line is a linear fit of the data. Cells were lysed with a 1% solution of Triton-X-100 in PBS. Upon centrifugation a pellet containing the nanoparticles was suspended with a 10 mM solution of imidazole and centrifuged once more. The fluorescence of the supernatant was measured at 485 nm excitation and 535 nm emission. The concentration was evaluated by the standard curve and the number of GFP molecules was estimated to be ˜10⁵ per cell.

FIG. 12. PPC-1 cells before and after near-infrared excitation. The black pixels seen in the ratio of the after and before indicates that there is no increased intensity from the autofluorescence of the PPC-1 cells in the green fluorescence channel. Scale bar is 50 μm.

FIG. 13. (a) Schematic of orthogonal HGN surface with two scaffolds presenting a protein of interest alongside a cell-targeting and internalizing peptide. The first scaffold that holds the protein cargo is the same as in FIG. 1a. An independent, second thiol-terminated polyethylene glycol (or thiol-DNA) linker is chemically linked to an internalizing peptide, here, poly-arginine labeled with a FAM fluorophores for visualization. The fluorophore is not needed for function, but for visualization only. (b-d) PPC-1, treated with HGN coated with two distinct polyethylene glycol scaffolds, one with a poly-arginine peptide labeled with a FAM fluorophore for internalization (b), and one with a Cy3 label. (d) Merged image of b and c demonstrating internalization of the co-stained nanoparticle within PPC-1 cells. This embodiment separates cell targeting and internalization from the protein cargo, enabling a wider variety of proteins to be delivered.

FIG. 14. HGN-siRNA-RP Hollow gold nanoshell (HGN)-siRNA-targeting peptide (RP) architecture with original or modular design and schematic of nanoparticle uptake, laser-activated siRNA delivery pathway in PPC-1 cells. (A) TEM image of HGN showing the hollow center surrounded by a higher contrast rim. (B) Schematic of the original HGN-SD-RP architecture and second-generation architecture for modular HGN-LD-RP to achieve versatile siRNA assembly. A fluorescein fluorescent label is added to both SD and LD to help quantify delivery. (C) Schematic of nanoparticle uptake and laser-activated siRNA delivery pathway. Endocytosis is promoted by the RP, RPARPAR. Femtosecond pulses of NIR light separate the nucleic acids (either SD or LD in (B) from the HGN at the Au—S bond, followed by vapor bubble formation as the light energy is converted to heat. The vapor bubbles burst the endosome, releasing the siRNA to the cytosol, allowing for gene silencing.

FIG. 15. Functional plk1-siRNA released from HGN-SD-RP by NIR-laser (2.4 W/cm² for 10 seconds) leads to loss of PPC-1 cell viability and down-regulation of PLK1 protein levels. (A) NIR-laser treatment of PPC-1 cells having internalized HGN-SD-RP (H) causes a significant decrease of cell viability similar to the effect of LIPOFECTAMINE (G), but at much lower RNAi concentration. A series of controls (defined in the text) are shown in the table underneath the growth curve. ***: p<0.001; ns: not significant. (B) Western blot analysis showing knockdown of plk1 gene expression in PPC-1 cells. Red boxes highlight the down-regulated expression of plk1 in cells with laser-released siRNA from HGN-SD-RP. The column graph underneath shows the band intensity ratio of PLK1 to (β-actin in Western blot image. The HGN-SD-RP provided the similar level of plk1 knockdown as LIPOFECTAMINE (Life Technologies, Thermo Fisher Scientific, Grand Island, N.Y.).

FIG. 16. NIR laser-activated release of siRNA from HGN-SD-RP in PPC-1 cells demonstrated by confocal fluorescence microscopy and flow cytometry. (A) Fluorescence microscopy (FAM channel) of HGN-SD-RP taken up by PPC-1 cells. HGNs are recognizable as bright dots associated with each cell mostly collecting around the perinuclear area. (B) The same area of the cell monolayer showed in A) after NIR pulsed laser irradiation efficiently releases HGN cargo. (C) Difference map of (A) and (B). Pre-laser and post-laser images are overlaid and color scale chosen such that purple indicates an increase, green a decrease, and gray scale no change in pixel intensity. Scale bar is 50 (D) Fluorescence intensity ratios of B) to A) for pixels in the selected four red regions. Blank 1 is the glass slide background outside the cells; Regions 2, 3 and 4 are drawn along the edge of the single cell outline, respectively. 40%-60% of pixels in regions 2, 3 and 4 have greater fold-increase in intensity than Blank 1 (Ratio ˜1), while the remaining areas where pixels show no increase are predominately nuclear regions. The expansion of bright pixels in perinuclear area post-laser suggests endosome release caused by laser irradiation. (E) PPC-1 cells were incubated with HGN-SD-RP then cell fluorescence intensity was assessed by flow cytometry before (red) and after (blue) pulsed NIR-laser treatment compared to fully untreated (no HGN-SD-RP, no laser) PPC-1 (black). Bars indicate the percentage of cells within the range of fluorescence intensity. Approximately 70% of the cell population showed a significantly enhanced fluorescence signal after particle internalization and laser release, defined by being above the brightest 1% of the unlabeled control cells.

FIG. 17. Targeted delivery of HGN-SD-RP. A) HGN-SD-RP are internalized into PPC-1 cells but not in RWPE-1. (A) PPC-1 cells show punctate fluorescence from FAM on HGN-SD-RP. RWPE-1 cells exposed to the same concentration of nanoparticles show none. Upper right inset: bright field; scale bars: 50 μm. (B) Flow cytometry assessment of PPC-1 and RWPE-1 cells fluorescence. Control indicates cells only. Cells+Particles indicates cells that were incubated with HGN-SD-RP. Only PPC-1 showed uptake, with bars indicating the percent of cells within the range of fluorescence. (C) siRNA delivered to RWPE-1 cells by Lipo induces decreased viability, indicating susceptibility to plk1 knockdown. RWPE-1 cells incubated with HGN-SD-RP following laser irradiation do not show any decrease in viability compared to untreated controls, likely due to lack of HGN-SD-RP internalization. Cells were plated into 96-well plates for cell viability assay at 24 hours, 48 hours, and 72 hours after laser release.

FIG. 18. Cell uptake and knockdown efficiency assessment of the nanocarrier with new modular design (HGN-LD-RP). A) HGN-LD-RP internalization detected by flow cytometry analysis of PPC-1 cells after incubation with HGN-LD-RP and laser treatment, as shown by the shift of the FAM intensity peak from the cell only control. Release is indicated by the intensity shift (compare blue and red). Bars indicate the percent of cells within the range of fluorescence intensity. B) PPC-1 cell viability assay 72 hours after laser treatment (2.4 W/cm² for 10 s) indicates siRNA release and RNAi from modular HGN-LD-RP. Cell viability is expressed relative to untreated cells (cell only).

FIG. 19. Determination of HGN particle concentration. (A) Size distribution and total concentration of HGN by particle tracking analysis using a Nanosight LM10HS (NanoSight Ltd., Amesbury, UK). (B) Correlation between measured HGN concentration and optical density at maximum absorption wavelength.

FIG. 20. HGN-SD-RP synthesis and characterization. (A) Schematic of the HGN-SD-RP synthesis steps. (B) Size distribution of nanoparticles for the steps during coating. HGN-citrate has a Z-average diameter of 56 nm; HGN-ssRNA 66 nm; hybridized HGN-dsRNA 73 nm; final construct HGN-dsRNA-RP 89 nm. (C) Absorption spectrum of HGN broadens and red-shifts along with the increase of particle size and size distribution range at each coating step. The plasmon peak shifted from original ˜710 nm to ˜800 nm after RNA and peptide coating.

FIG. 21. Native-PAGE and densitometry analysis of chemically released siRNA (SD) from HGN. (A) Native-PAGE gel of siRNA KCN-released from HGN (lanes 1-4, replicates from the same sample), and calibration siRNA concentration gradient (lanes 5-9). Concentration gradient used, from lane 5 to lane 9: 3 pmol, 2 pmol, 1 pmol, 0.5 pmol, and 0.2 pmol. (B) Calibration curve correlating known siRNA concentration and band intensity from densitometry measurement. The number of siRNA short duplex strands per HGN is estimated from the linear fit equation to be 2300±600.

FIG. 22. Comparison of siRNA coating density between SD and LD on HGN. (A) Fluorescence intensity of KCN-released SD siRNA was measured and the contribution per HGN calculated. This value was set to 1. KCN-released LD per HGN was estimated as ˜60% of the SD. (B) Native-PAGE of KCN-released siRNA from HGN. Lane 1: KCN-released SD; lane 2: KCN-released LD. Lack of ssRNA (23 nucleotides) in lane 1 suggests ˜100% hybridization efficiency for SD, however, an apparent band of ssRNA in lane 2 shows less hybridization efficiency for LD formation, supporting the fluorescence-based data.

FIG. 23. NIR laser-dependent release of siRNA from HGN. A) Laser release efficiency of HGN-SD-RP with 10 s pulsed laser treatment (1.3, 2.4, 4.0 W/cm²) compared to KCN chemical release (taken to be 100%). Q dye is on the 5′ of the anti-sense strand. 2.4 W/cm² for 10 s laser irradiation was found to release ˜85% SD on HGN. B) Native PAGE analysis of released RNA. Lanes 1 and 2 show dsRNA released with laser treatment at 4.0 W/cm² for 10 s. The lack of ssRNA (23 nucleotide length) upon laser treatment suggests the duplex RNA remains hybridized.

FIG. 24. Q modification on 5′ end of anti-sense strand in PLK1-siRNA blocks the siRNA knockdown activity, as indicated by cell viability. A) Different siRNA structures with or without Q at different sites on the strands. B) Cell viability assay 72 h after LIPOFECTAMINE (Life Technologies, Thermo Fisher Scientific, Grand Island, N.Y.) transfection of different siRNA options. Second generation of siRNA architecture shows similar knockdown efficiency as original short duplex structure. Q modification on 5′ end of siRNA anti-sense strand blocks the siRNA knockdown activity (C, D and F). However, both thiol-PEG and Q modifications on 5′ end of siRNA sense strand do not (B, E and G), indicating the importance of anti-sense strand 5′ site for siRNA functionality.

FIG. 25. NIR laser power optimization for effective siRNA release from HGN surface with minimum cell damage caused by particle local heating. PPC-1 cells incubated with either HGN-SD-RP or HGN-dsDNA-RP are exposed to different laser power intensity and irradiation duration combinations. Cell viability is assayed 72 hours after laser treatment. 2.4 W/cm² for 10 s was chosen as optimal for effective siRNA knockdown. Minimal loss of cell viability was found for all HGN-dsDNA-RP conditions, indicating minimal damage induced by the local heating.

FIG. 26. HGN-siRNA-RP dosage titration for effective siRNA knockdown in PPC-1 cells. Various concentrations of HGN-siRNA-RP were incubated with PPC-1 cells, treated with laser, and then plated to 96-well plate to assay cell viability after 72 hours. We found 6.5 pM (for 2×10⁵ cells) to be the minimal dosage for effective siRNA knockdown and cell apoptosis.

FIG. 27. Nanoparticle architecture and characterization of siRNA on HGN, and schematic of nanoparticle uptake, NIR laser-activated siRNA delivery and RNAi-mediated differentiation in hESC. (a) Schematic of the HGN-siRNA-TAT architecture, NIR laser-activated siRNA delivery and RNAi-mediated differentiation in hESC. Q: Quasar570. (b) Size distribution and absorption spectra of nanoparticles during the coating steps. HGN-citrate has a Z-average diameter of 56 nm; HGN-siRNA-NH₂, 95 nm; HGN-siRNA-Biotin, 105 nm; HGN-siRNA-Biotin-STV, 147 nm; and final construct HGN-siRNA-Biotin-STV-TAT, 151 nm. Upper right of the left panel: TEM image of the final construct. Scale bar: 50 nm.

FIG. 28. GFP knockdown in H9-GFP cells via HGN-mediated GFP-siRNA delivery and NIR-laser excitation (2.4 W/cm² for 15 s). (a) Laser power and irradiation duration optimization for effective GFP knockdown in H9-GFP cells. Cells are assayed by flow cytometry three days after laser treatment. (b) Fluorescence imaging of H9-GFP cells three days after HRT (coated with GFP-siRNA) internalization and laser treatment. (c) Mean fluorescence intensity of cells with and without HRT and HDT (dsDNA control) and laser treatment. Cells are assayed by flow cytometry three days after laser treatment. Lipo: Lipofectamine RNAiMAX siRNA transfection. Data sets are analyzed by one-way ANOVA. **, p<0.01; ***, p<0.001; NS, not significant.

FIG. 29. Cellular uptake of HRT in un-engineered hESC and cytotoxicity assay of the particle and pulsed NIR laser treatment. (a) Microscopic visualization of particles internalized by hESC H9 cells. Left: Dark-field imaging of cells shows gold punctate dots due to HGN light scattering, which are co-localized with red fluorescent puncta (from Quasar570) surrounding the nucleus (see middle panel). Right: 3D image of a single cell by confocal fluorescence microscopy shows nanoparticles (red puncta from the fluorescent dye Quasar570) collecting around the nucleus (blue by Hoechst staining). (b) Flow cytometry quantification of particle internalization and laser release in H9 cells. HRT(Lipid): HGN-siRNA-TAT by lipid coating strategy. Top right: mean fluorescence of each peak showing increased intensity after laser treatment that indicates the release of fluorescent payload. (c) HRT is efficient (>97%) in penetrating a series of different hESC cell lines including H1, H7, H9 and H14. The bar is defined as being above the brightest 1% of the unlabeled control cells. (d) Cell viability assessment of H9 cells after internalization of HRT (with dsRNA coating non-sense to H9 cells) and treatment with different laser powers. No significant difference was observed from the t-test analysis between cells without HRT without laser and cells with HRT with laser irradiation at 2.4 W/cm² for 15 s. Top panel: cell colonies stained by crystal violet 5 days post laser treatment.

FIG. 30. Release of Oct4-siRNA from HRT in H9 hESC cells by NIR laser (2.4 W/cm² for 15 s) down-regulates OCT4 protein levels and leads to stem cell differentiation in the mTeSR1 medium. (a) Time schedule of the whole protocol and assays of particle and laser treatment to cells. (b) ICC staining of H9 5 days after particle internalization and laser treatment. (c) Flow cytometry of H9 cells stained with stem cell markers including OCT3/4 and TRA-1-60 five days post particle and laser treatment in mTeSR1 medium, compared to the undifferentiated cells in controls (hESC only, HRT without laser and HDT with laser). Bars are defined as being above the brightest 1% of the unlabeled control cells, incubated with secondary antibody but without primary antibody. (c) Western blot analysis of OCT4 protein level in H9 cells. The bar graph underneath shows the band intensity ratio of OCT4 to (β-actin in the Western blot image. (e) Morphology of cells five days post HRT or HDT and laser treatment (cultured in the mTeSR1 medium).

FIG. 31. The HGN and NIR laser-mediated Oct4 gene knockdown accelerates the ability of hESC H9 cells to differentiate into all three germ layers in differentiation medium, indicated by ICC staining and RT-PCR analysis of differentiation markers. (a) ICC of βIII-tubulin (TUBB3, ectoderm marker), α-smooth muscle actin (α-SMA, mesoderm marker), and α-fetoprotein (AFP, endoderm marker) for cells 20 or 28 days (20 for α-SMA, 28 for TUBB3 and AFP) after particle and laser treatment. (b) RT-PCR analysis of differentiation biomarkers in H9 cells 19 and 21 days after particle and laser treatment. MAP2: ectoderm; BRACHYURY: mesoderm; FOXA2, AFP, CDX2: endoderm.

FIG. 32. Release of siRNA from HGN-siRNA-TAT with pulsed laser treatment at different powers (2.4 and 4.0 W/cm² for 10 seconds, 15 seconds, or 20 seconds) compared to KCN chemical release (100%). Quasar570 dye is on the 5′ of the antisense strand. Laser irradiation at 2.4 W/cm² for 15 seconds releases ˜23% of the total siRNA loading on HGN.

FIG. 33. Addition of ROCK inhibitor after single H9 cell seeding significantly enhances the stem cell viability. (a) ROCK inhibitor treatment after seeding for 24 hours significantly preserves cell number. Under phase contrast light microscopy, cells are observed to elongate with the ROCK inhibitor addition and shorten back when removed. (b) Crystal violet staining of colonies after 6 days affirms the robustness of ROCK inhibitor treatment.

FIG. 34. Flow chart describing an exemplary transfection protocol of hESCs with siRNA-carrying particles and subsequent NIR laser treatment. Warm PBS dissociation of H9 cells and ROCK inhibitor addition before cell seeding increase stem cell viability.

### DETAILED DESCRIPTION OF ILLUSTRATIVE EMBODIMENTS

This disclosure provides compositions and methods that allow one to deliver a cargo molecule such as a protein, polypeptide, or polynucleotide into a cell. In some cases, the compositions and methods further allow one to have precise spatial and/or temporal control of the delivery of the cargo molecule.

In one aspect, this disclosure describes a modular nanocarrier platform that allows intracellular delivery of a peptide. Proteins signal, perform, and/or regulate functions of all living cells including, for example, enzymatic catalysis, signal transduction, gene regulation, and apoptosis. When protein concentration or function goes awry or is altered, disease can result. Thus, protein-based therapies can be a component of treatments for cancer, vaccination, inflammation, and/or regenerative medicine. A first step toward protein therapeutics is delivery of functional proteins to cells in culture. A protein delivery system could be used to deliver protein to, for example, an entire monocell culture, specific cell types within a culture that possesses a mixture of cell types, and/or individual cells within a mono- or mixed culture with high throughput, while maintaining high efficiency, low toxicity, and control over the timing of delivery.

Protein delivery can be confounded by sensitivity of protein function to amino acid sequence, secondary structure, and/or tertiary structure. To reach an intracellular site of action, a protein must cross the cell membrane without damaging either the protein or the cell and reach the cytosol without being permanently trapped and/or degraded within endosomes or lysosomes. The ‘last mile’ problem—i.e., a tendency for a protein to become trapped inside endosomes—has been approached using protein transduction domains, endosomal disrupting agents, cytosolic degradable polymeric carriers, and/or membrane interacting supramolecular particles.

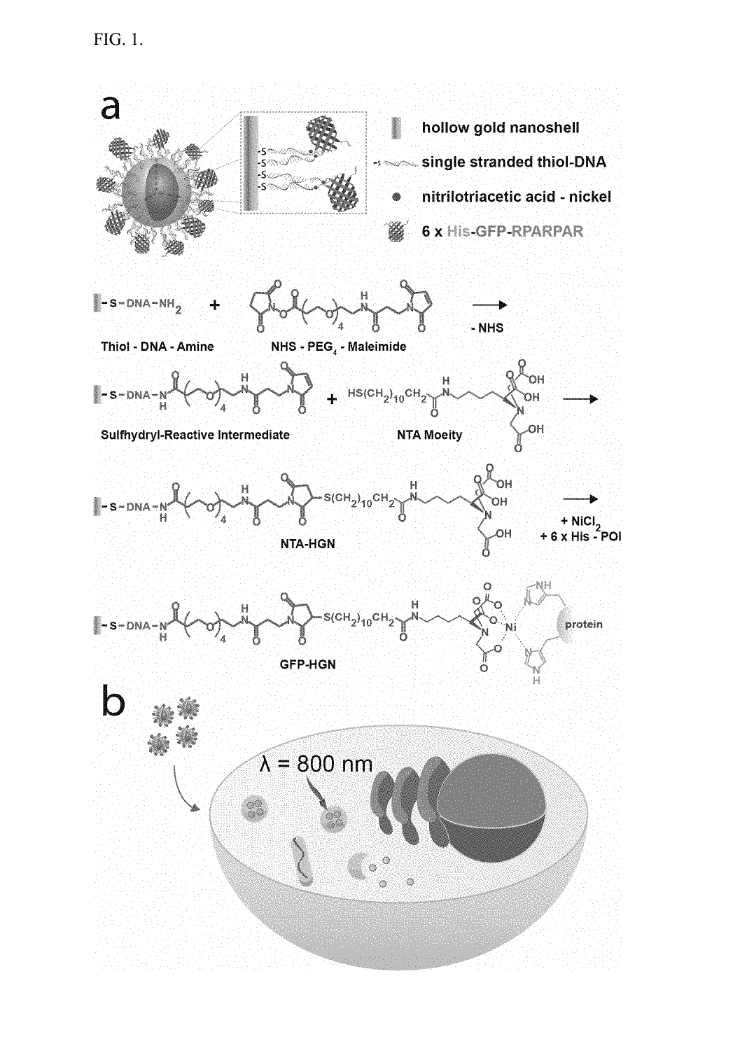

In contrast to conventional delivery technologies, this disclosure describes a modular nanocarrier platform capable of carrying a cargo protein and a cell internalizing signal. Endosome escape and intracellular release are triggered by femtosecond pulses of near-infrared-light (FIG. 1). This new delivery vehicle can target individual cells in culture with the same resolution as microinjection, while also being capable of delivering protein to an entire cell culture, or to specific cell types in a mixed culture at high throughput and high efficiency with low toxicity. Sub-micron to millimeter scale cytosolic patterning of proteins not amenable by any other transfection method is possible. Moreover, individual cells can be rapidly targeted using a conventional two-photon microscope in real time.

In some embodiments, the cargo protein can include a particular tag sequence. Tag sequences have been used for efficient purification, detection, and/or immobilization of many varieties of proteins, especially recombinant proteins. One commonly used tag is the histidine (His) tag, which typically consists of five or six His residues added to the N- or C-terminus of the protein. The imidazole moieties of the His residues (FIG. 1) chelate the free coordination sites of metal ions, which in turn can be immobilized as chelate complexes of nitrolotriacetic acid (NTA) bound to a solid support via various linkers (FIG. 1).

Nickel (Ni⁺²) and other divalent metal ions (e.g., Cu⁺², Co⁺², Zn⁺²) chelated to NTA are used in chromatographic media for immobilized metal affinity chromatography and in protein sensors. The affinity of individual NTA-Ni-His interactions is relatively low, with an estimated K_{D} of 10′ at neutral pH. However, in the context of chromatography applications, the support can possess a high density of immobilized Ni-NTA sites. The multiple linkages and efficient “rebinding” are possible between the support and the protein, providing net high affinity and long-lasting binding between the protein and the support for chromatographic separations. Two or more Ni-His bonds are necessary for stable binding. In affinity chromatography and biosensors, the protein can be eluted from the support by the competitive complexation of the metal with free imidazole.

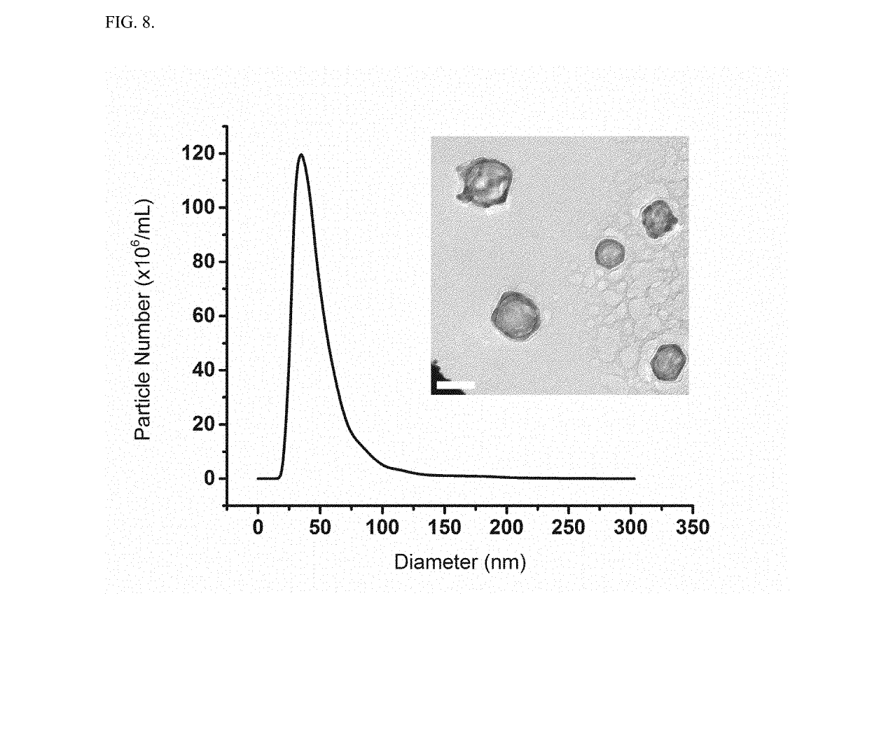

The cytosol protein delivery system described herein can involve the use of a NTA-Ni linker to attach His-tagged proteins, using hollow gold nanoshells (HGN) as the solid support. In some embodiments, hollow gold nanoshells of 40±15 nm in diameter and 5±2 nm in shell thickness were synthesized via the galvanic replacement of gold from sacrificial silver templates as described previously (Braun et al., 2009, ACS Nano 3:2007-2015; Prevo et al., 2008, Small 4:1183-1195; Wu et al., 2009) to optimize the plasmon absorption peak at ˜800 nm (FIG. 8). In such embodiments, a monolayer of single stranded thiol-DNA-amine can be assembled onto the hollow gold nanoshell surface using, for example, low-pH adsorption (Zhang et al., 2012, J Am Chem Soc 134:7266-7269) to stabilize the hollow gold nanoshells against aggregation in saline, while providing a high density linker that can be functionalized via the terminal amine (Hurst et al., 2006, Analytical chemistry 78:8313-8318). To determine the packing density of the thiol-DNA-amine, an NHS-fluorescein dye was attached to bind to and label the amines. The fluorescence intensity was consistent with ˜3000 DNA strands per 40 nm diameter hollow gold nanoshell.

To create the protein handle, N—[Nα,Nα-Bis(carboxymethyl)-L-lysine]-12-mercaptododecamamide (NTA Moeity in FIG. 1(a)) was attached to the thiol-DNA-amine bound to the hollow gold nanoshell via a flexible polyethylene glycol thiol to amine linker, NETS-PEG-Maleimide. The PEG linker combined with the dodecamide provides significant freedom to the NTA-Ni to allow binding to the His residues on the protein. A densely packed NTA-Ni surface can provide the polyvalent Ni-His interactions needed to bind the hollow gold nanoshell to the His-tagged protein.

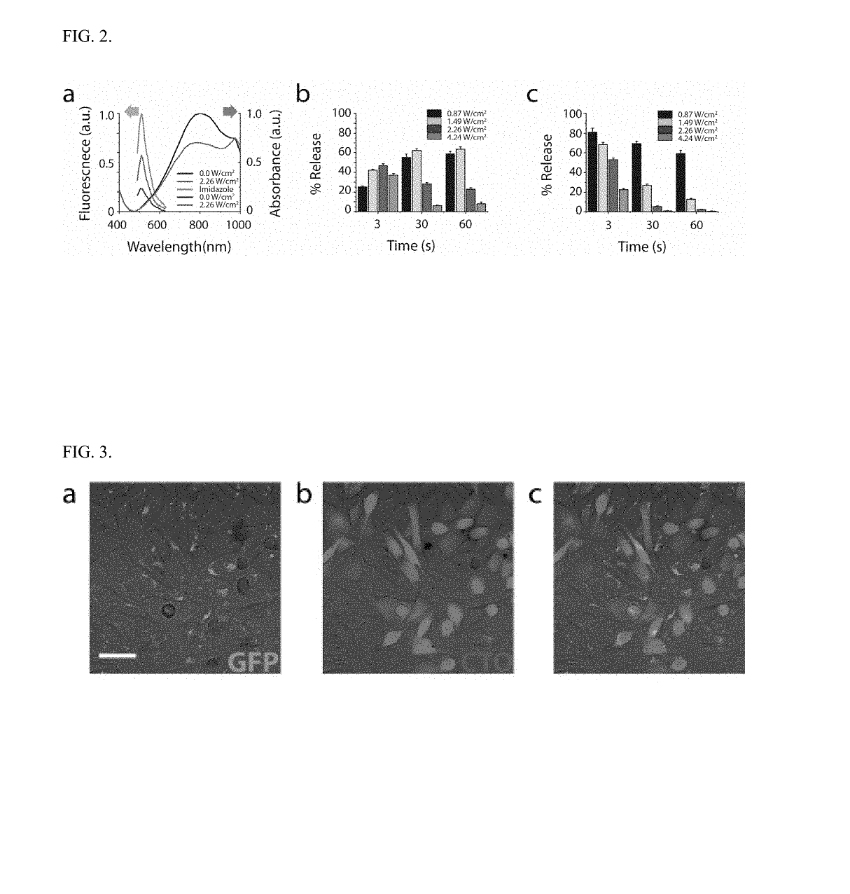

Green fluorescent protein (GFP) was used as a model cargo protein. The GFP was modified to include a 6-His-tag on the N-terminus and a RPARPAR (SEQ ID NO:1) cell targeting peptide on the C-terminus that facilitates bonding to neuropilin-1 on PPC-1 cells, which promotes cell internalization. 6-His-labeled GFP was loaded onto hollow gold nanoshells at a 100,000 to 1 GFP:HGN molar ratio in the presence of 400 μM NiCl₂. The protein loading and release were monitored through distance-dependent quenching of GFP fluorescence in proximity to gold nanostructures (Bajaj et al., 2010, Chemical Science 1:134; Cheng et al., 2011, Anal Chem 83:1307-1314). The fluorescence of the GFP loaded on the hollow gold nanoshells was reduced to ˜20% compared to the native (unquenched) fluorescence (FIG. 2a).

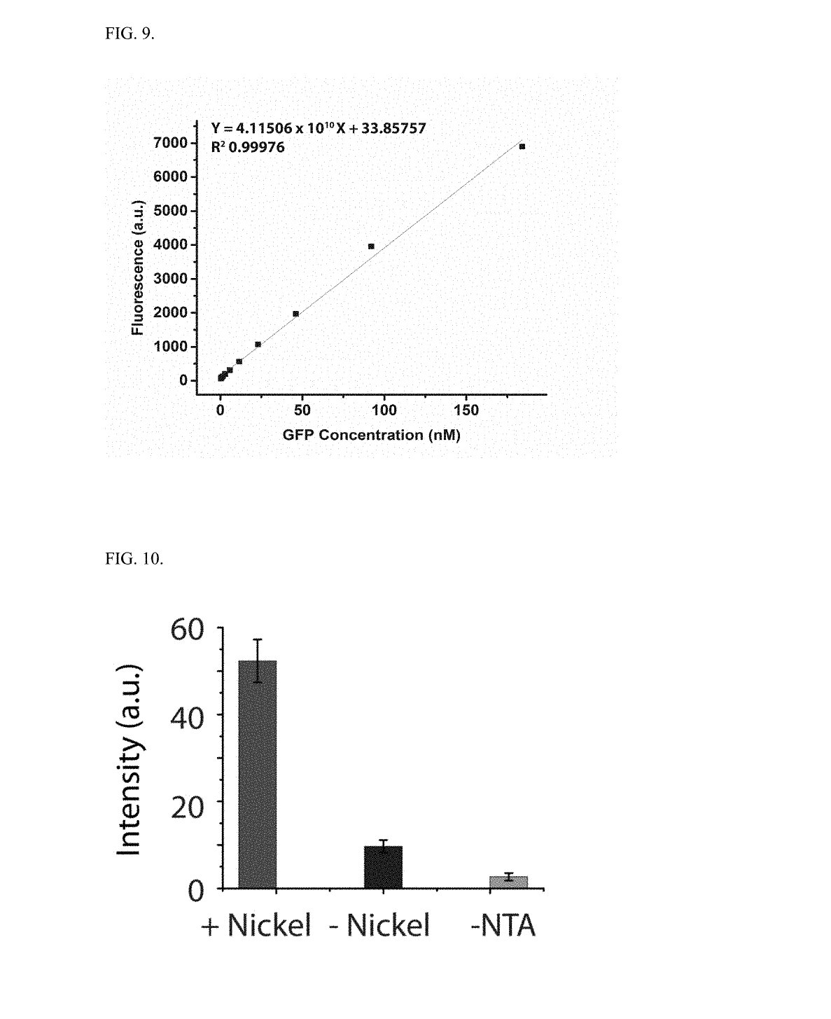

When a His tag is used in affinity chromatography, the bound protein is released by adding imidazole, or a similar agent, that bind more strongly than His to the Ni²⁺. However, one cannot add imidazole to the cell interior to release the His-tagged protein. Instead, a femtosecond-to-picosecond pulse of near-infrared light can cleave the thiol bond holding the DNA linker to the hollow gold nanoshell. Fast pulses can confine the light energy absorbed to the HGN before thermal dissipation can equilibrate the HGN temperature with its surroundings. This can cause the effective HGN temperature to increase hundreds of degrees, which can induce cleavage of the thiol-gold bond holding the linker to the gold, without damaging the linker or the protein. Once the NTA-Ni is freed from the hollow gold nanoshell, each linker acts on its own with the much lower K_{D} of the individual NTA-Ni-His bond. Thus, the linker moieties dissociate rapidly and diffuse away, leaving the protein free and in its active state. One feature of using near-infrared light to induce release is that cells in culture, as well as tissue, blood, etc. are relatively transparent to 650-900 nm wavelength light, allowing near-infrared transmission in soft tissues at depths up to several cm. The use of NTA-Ni therefore provides a modular platform to facilitate cytosol delivery of a wide variety of His-labeled proteins. Moreover, the system exhibits minimal non-specific binding in the absence of nickel ions (FIG. 9 and FIG. 10).

The extent of GFP binding on the HGN can be determined by the competitive adsorption of imidazole for the NTA-Ni sites and measuring the increase in GFP fluorescence relative to a standard. Approximately 800 GFP can bind per 40 nm diameter hollow gold nanoshell, which is equivalent to approximately 1 GFP per 6-8 nm² if binding only occurs to the exterior of the hollow gold nanoshell. GFP is a barrel-shaped protein 4.2 nm long and 2.4 nm in diameter, and has a cross-sectional area of approximately 4.5 nm². If each of the approximately 3000 DNAs that are bound to the hollow gold nanoshell is also bound to an NTA-Ni, there are about four NTA-Ni for every GFP.

FIG. 2a shows that when GFP-HGN was irradiated by pulsed near-infrared light (130 fs pulses, 1 KHz repetition rate), the GFP fluorescence increased in a power-dependent and exposure-time-dependent fashion, signifying detachment of the GFP from the hollow gold nanoshells. The plasmon peak at 800 nm also decreased with increased laser power and exposure time as the stabilizing surface ligands were removed and the hollow gold nanoshells aggregated and annealed into solid gold nanoparticles (FIG. 2a). Laser excitation at 800 nm achieved a maximum 63% release compared to imidazole at a power of 1.5 W/cm² (FIG. 2b), which indicates significant polyvalent NTA-Ni-His attachments between the hollow gold nanoshells and the GFP.

Without wishing to be bound by any particular theory of operation, all but one of the thiol-DNA bonds holding the DNA to the hollow gold nanoshell can be broken for the GFP to release and, consequently, for GFP fluorescence to increase. Two or more remaining bonds can be sufficient to hold the GFP to the hollow gold nanoshell. Decreasing amounts of GFP remaining on the hollow gold nanoshells after adding imidazole to irradiated nanoparticles in FIG. 2c indicates significant shedding of the surface ligands (and GFP) by laser not reflected in FIG. 2b. Even after 60 s at 1.5 W/cm² fluency, however, only 10% of the GFP remained on the hollow gold nanoshells despite 63% release by laser. The decrease in GFP quantities after laser excitation of plasmonic nanoparticles in the presence of fluorescent proteins may be due, at least in part, to absorption of GFP onto freshly exposed gold surfaces. This observed ratio of NTA-Ni to GFP provides good stability during synthesis, purification, and delivery, and an acceptable release rate on laser excitation.

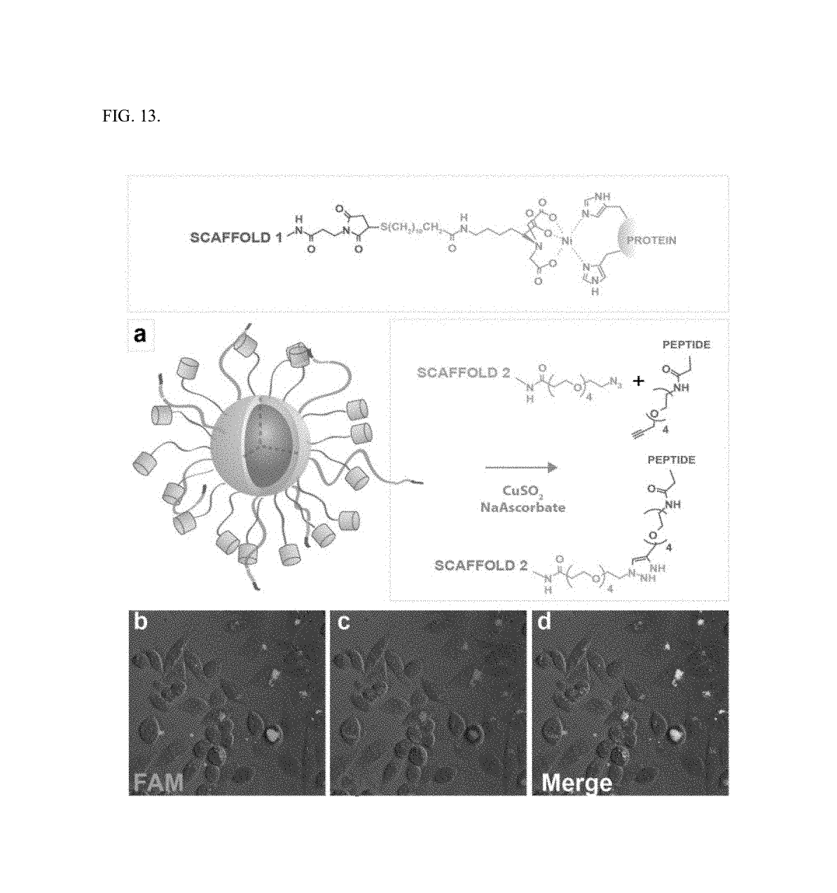

In some embodiments, orthogonal construction is used, which employs thiol-gold chemistry to attach two scaffolding units to the HGN (FIG. 13a). In the embodiment illustrated in FIG. 13, the scaffolds are linked to the HGN via two independent, twenty-five nucleotide thiol DNA with an amine or azide functional group at the opposing terminus. In alternative embodiments, 3 kD thiol-PEG (polyethylene glycol) can be used. FIG. 13 shows an embodiment in which Scaffold 1 includes a thiol-DNA-amine (Integrated DNA Technologies, Inc., Coralville, Iowa) and Scaffold 2 includes a thiol-DNA-azide (Integrated DNA Technologies, Inc., Coralville, Iowa) or synthesized from a thiol-DNA-amine via amine reaction with NHS-PEG4-Azide (Pierce, Thermo Fisher Scientific Inc., Rockford, Ill.). Alkyne-derived peptides for “Click” chemistry can be prepared by the reaction of alkyne-PEG4-maleimide with the sulfur group of a dye labeled peptide (LifeTein LLC, Hillsborough, N.J.) on a cysteine residue. The peptide can be characterized by HPLC and mass spectrometry. High-density coupling of Scaffold 1 and Scaffold 2 to the HGN can be performed by low pH-induced adsorption at determined ratios. The NTA moiety, N—[Nα,Nα Bis (carboxymethyl)-L-lysine]-12-mercaptododecanamide (Sigma-Aldrich, St. Louis, Mo.), can be linked to Scaffold 1 via a NHS-PEG4-maleimide linker (Quanta Biodesign, Ltd., Powell, Ohio) (FIG. 13a). The alkyne-peptide is attached to the nanoparticle via a “click” reaction with the azide on the scaffold with a Cu(I) catalyst (FIG. 13a) (65). Fluorescence labeling of the key functionalities throughout the assembly can be used to quantify loading and efficiency of reaction. FIGS. 13b-d show preliminary results using this approach; the dyes FAM and Cy3 are located on different scaffolds on a given HGN and show co-localization after delivery to PPC-1 cells.

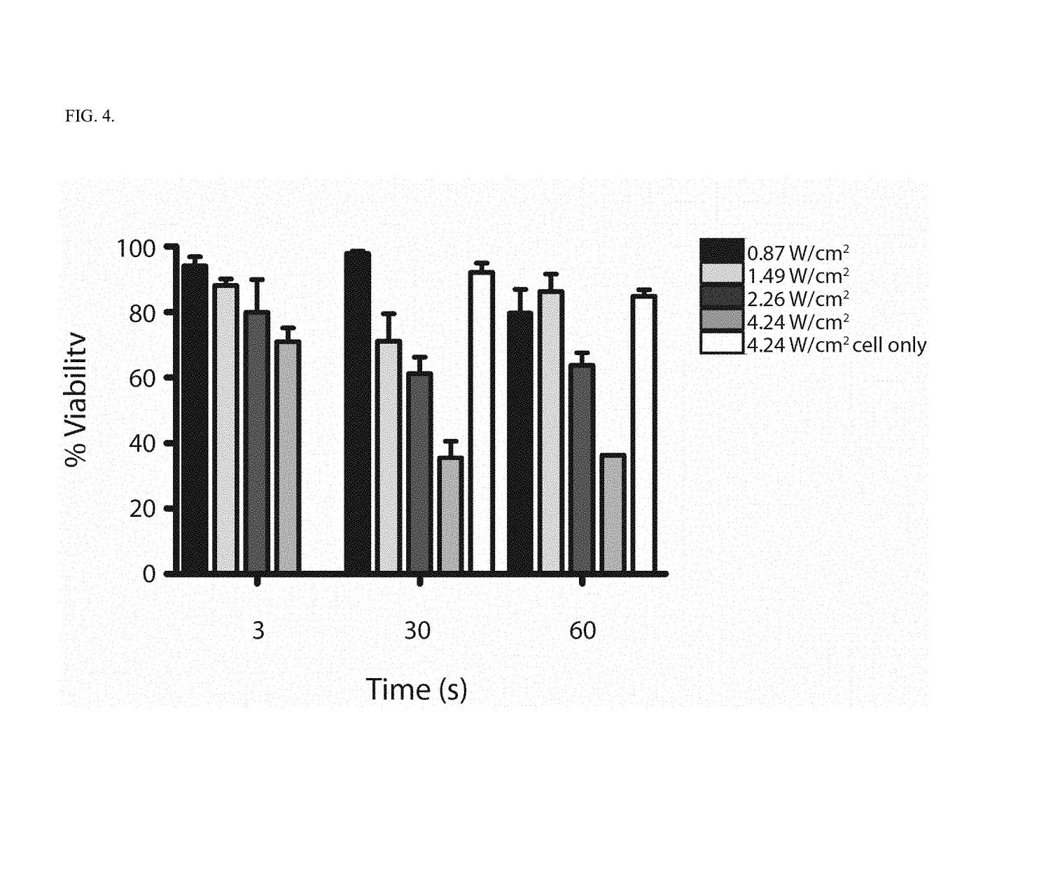

A cell-penetrating variant of GFP with the peptide sequence RPARPAR (SEQ ID NO:1) on the C-terminus was constructed to facilitate rapid, cell-specific endocytosis of the HGN-GFP construct to achieve PPC-1 cell specificity. The RPARPAR sequence, when fused to a protein at the C-terminus, follows the C-end rule (CendR) to bind specifically to the neuropilin-1 (NRP-1) receptor that is over-expressed in certain types of cancer cell lineages, including the PPC-1 prostate cancer epithelial cells used as a model target cell in our study. The NRP-1-deficient cell line M21 served as a negative control. PPC-1 and M21 cells were co-cultured on a chambered glass slide to assess the internalization and specificity of the RPARPAR GFP-HGN construct (FIG. 3a). M21 cells were pre-stained with CellTracker orange (CTO) (FIG. 3b). Internalization of RPARPAR GFP-HGN was verified by a green punctate fluorescence found primarily within the cell boundary that was excluded from the nucleus, suggesting RPARPAR GFP-HGN localization within endosomes.

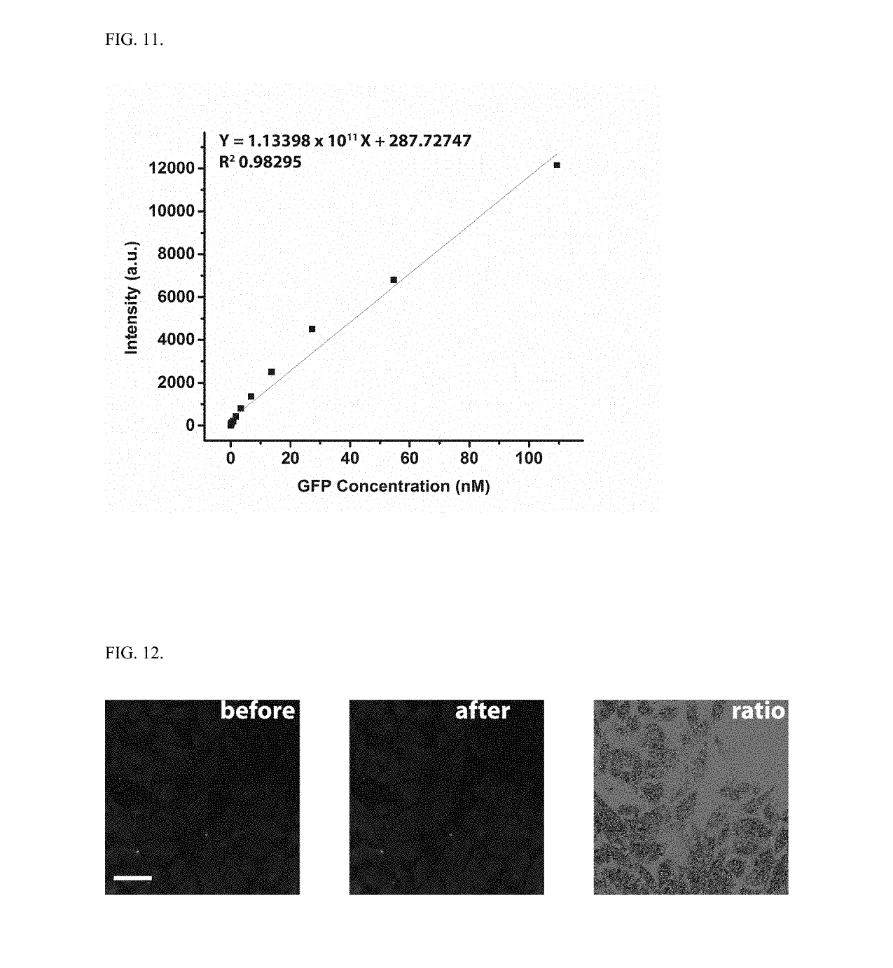

Nearly all the PPC-1 cells took up the RPARPAR GFP-HGN, and minimal RPARPAR GFP-HGN was found in the interior of the M21 cells (FIG. 3c). RPARPAR GFP-HGN internalized within a monoculture of PPC-1 was quantified by cell lysate fluorescence to be approximately 10⁵ GFP molecules per cell, about 200 RPARPAR GFP-HGN per cell (assuming a 63% release with 800 GFP per hollow gold nanoshell), or approximately 100 nM GFP, assuming a 2 pL cell volume (FIG. 11). Exposure of a monoculture of cells within a 0.32 cm² surface area with variable laser exposure power and time demonstrated minimal changes in cell viability within parameters below 4.2 W-cm′ and 30 s (FIG. 4), which exceeds the light power density required for GFP release (FIG. 2b and FIG. 2c).

Spatial and temporal targeting of protein release was explored using a conventional, two-photon microscopy system (FLUOVIEW 1000, Olympus America, Inc., Center Valley, Pa.). The two-photon microscope uses a tunable (690 nm-1020 nm) femtosecond pulsed laser that shares the working objective with a continuous wave blue (473 nm) laser scanning confocal microscope. The instrument allows switching between these two modes of excitation, with fine control over imaging and sub-micron resolution in laser patterning. PPC-1 cells were cultured on chambered glass slides, treated with GFP-HGN and imaged by confocal single-photon mode microscopy.

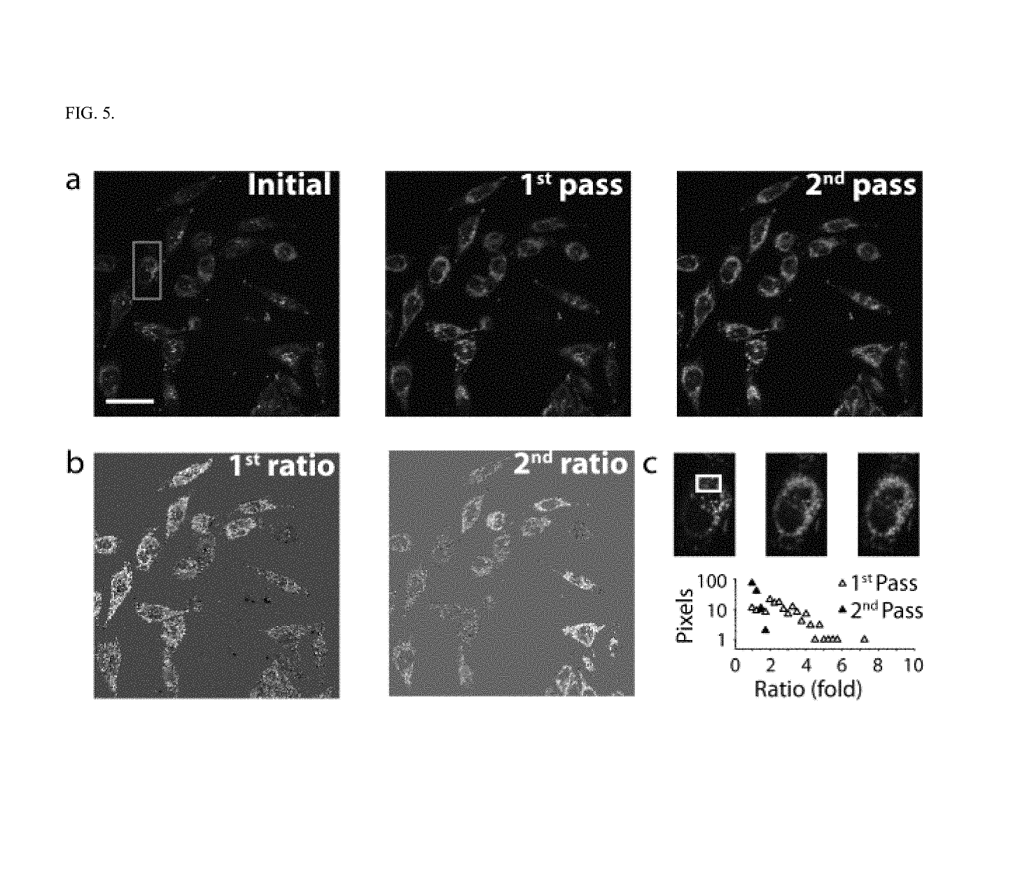

The sample was irradiated with a femtosecond pulsed laser at 800 nm wavelength, switching back to single photon imaging to compare with the initial image. This strategy was used at increasing powers of the pulsed-NIR laser to define a working range for GFP release, where the GFP pattern was found to change from the initial punctate spots into a more diffuse fluorescence in the exposed cells, with increased intensity due to dequenching of GFP (FIG. 5a). Notably, exciting the PPC-1 cells for repeated cycles released additional GFP from the hollow gold nanoshells, with diminishing returns (FIG. 5b and FIG. 5c) showing an exposure time dependency of release and exhaustion of the carrier. Exciting the PPC-1 cells without RPARPAR GFP-HGN reveals no increase in the autofluorescence of the cell upon exposure to pulsed-NIR laser (FIG. 12).

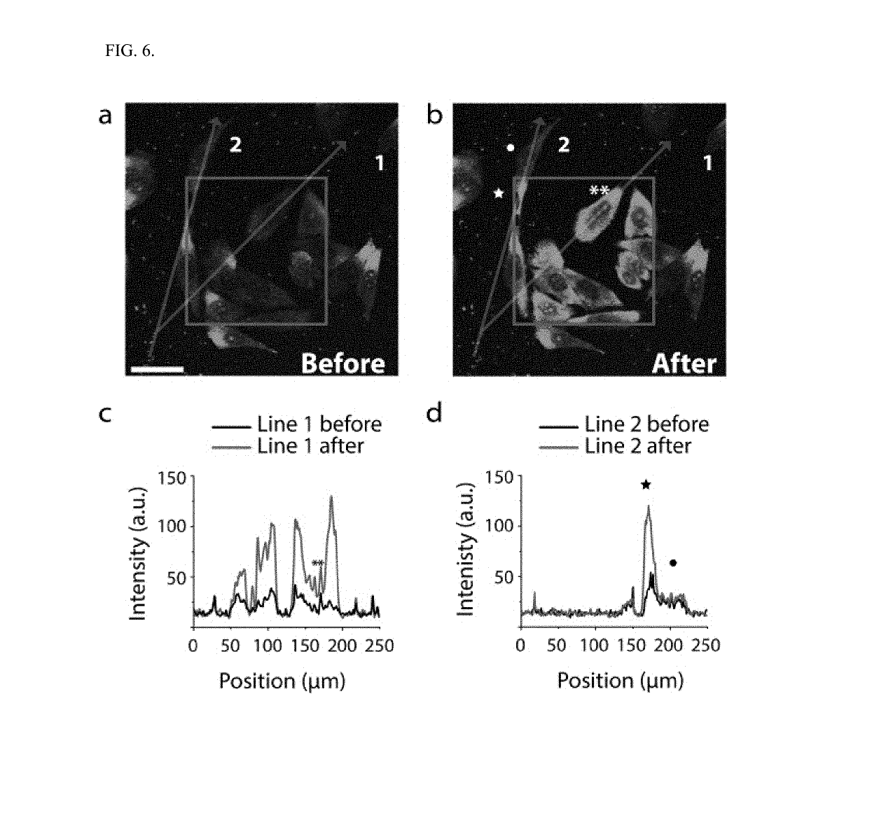

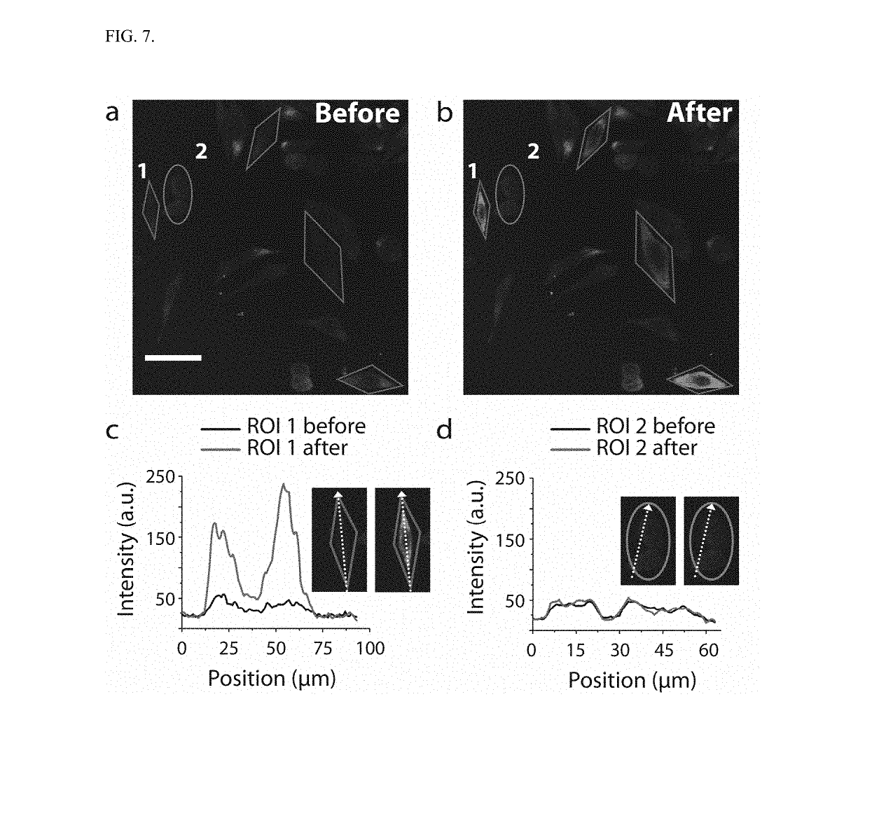

To explore the spatial release of the GFP in greater detail, a subset of PPC-1 cells within a monolayer was defined to be activated by focused laser irradiation. This subset of cells was interspersed between cells that would not be exposed. Rastering within a user-defined shape, the instrument modulates the excitation path using a beam deflector with precise control over exposure time at the pixel (sample volume) level. Single photon imaging defined the baseline fluorescence of the cells, and indeed some cells were initially brighter than others, attributed to varying uptake of the RPARPAR GFP-HGN (FIG. 6a). Near-infrared light was used to expose areas confined within each targeted area (red polygons). Single photon confocal imaging of GFP post-treatment revealed increased intensity for only the cells within the targeted area (FIG. 6 and FIG. 7). Furthermore, regions outside the targeted area showed no more than negligible change in fluorescence (FIG. 6d and FIG. 7d). This subcellular controlled release offers the ability to create internal protein gradients. Interestingly, the RPARPAR peptide (SEQ ID NO:1) on the GFP imparts some nuclear targeting once the protein is cytosolic, with nucleoli increasing in brightness after the laser release (FIG. 6b). Thus, the hollow gold nanoshell delivery system described herein provides the ability to deliver nuclear-targeted cargo—e.g., transcription factors—with cellular resolution.

In one aspect, therefore, this disclosure demonstrates spatially and temporally controlled cellular delivery of a model protein, GFP, from a plasmonic nanocarrier using a pulsed near-infrared laser. The design and assembly of the nanoparticle is modular and therefore can be readily tailored to alternative applications by substitution of components suitable for any desired application. For example, the NTA-Ni handle can be tailored for polyvalent attachment of genetically engineered poly-histidine tagged proteins. As another example, the thiol-DNA-NTA anchoring system can confer reproducible control over laser release—e.g., the gold-thiol bonds are thermalized and released by the near-infrared laser pulses. This reduces the valency of the non-covalently attached protein so that it is released from the NTA-Ni handle in a laser power and exposure dependent manner. Endosome escape is accomplished via vapor bubble formation from the hot hollow gold nanoshell which ruptures the endosome. One can target delivery of a cargo polypeptide to a specific cell type by, for example, designing a fusion protein that includes a cell internalizing peptide that is specific to the targeted cell type.

A model construct including GFP as a model cargo polypeptide with a 6-His tag on the N-terminus and the cell internalizing peptide RPARPAR (SEQ ID NO:1) on the C-terminus was successfully delivered to the cytosol of PPC-1 cells that express NPR-1, while no GFP was observed in NRP-1 deficient cell line M21. GFP release from the hollow gold nanoshell carrier, the NTA-Ni chelator, and the endosome was initiated by femtosecond pulses from a near-infra-red laser. Diffuse GFP fluorescence emission from throughout the cell confirmed both cell-specific uptake in co-cultures and enabled visualization of spatial and temporal release. Furthermore, a commercial two-photon microscope provided control and real-time monitoring of payload release by “painting” individual cells with near-infrared light, providing protein delivery with cell-level resolution with no additional equipment or effort. This technique allows one to deliver a wide range of proteins with direct access to the cytosol to cells, either individually or in concert with the delivery of one or more additional proteins. The RPARPAR internalization peptide (SEQ ID NO:1) also shows some delivery of GFP to the nucleus. Other targeting peptides, proteins, and cell lines can be used in methods that exploit the universal nature of the NTA-Ni-His linkage, the near-infrared triggered release of the protein from the nanocarrier, vapor-bubble induced endosome rupture.

While described above and illustrated in the context of using a hollow gold nanoshell (HGN) as the carrier, in alternative embodiments the carrier may be any suitable carrier. A suitable carrier may include any solid substrate to which the irradiation-cleavable linker may be attached. Exemplary alternative carriers include, for example, a CuS nanoparticle, a silver or gold nanocube, a silver or gold nanoshell, or a solid silver or gold nanoparticle. Thus, unless used in the context of describing a specific exemplary embodiment, reference to a thio-gold bond is merely exemplary and is used for brevity to describe what, in certain embodiments, can be a thio-silver bond or thio-copper bond. Also, reference to a hollow gold nanoshell or use of the HGN as an abbreviation therefore, is merely exemplary and used for brevity to describe what, in certain embodiments, can be a carrier that includes any of the suitable carriers listed in this paragraph.

Thioloated polyethylene glycol (FIG. 13), thiolated RNA, or thiolated DNA can be used to create an alternative linker between the nanoparticle surface and the NTA-Nickel protein coordination site. In addition, any moiety capable of connecting to the nanoparticle via the thermally labile thiol-gold bond may be used as a linker. In many embodiments, the linker can be easily released by NIR light. In some embodiments, an alternative linker also may sterically stabilize the nanoparticles against aggregation in saline.

While described above and illustrated in the context of using green fluorescent protein (GFP) as the cargo polypeptide, in alternative embodiments the cargo polypeptide may be any suitable polypeptide. “Polypeptide” refers to a sequence of amino acid residues without regard to the length of the sequence. Therefore, the term “polypeptide” refers to any amino acid sequence having at least two amino acids and includes full-length proteins, fragments thereof, and/or, as the case may be, polyproteins.

Suitable cargo polypeptides include, for example, any polypeptide that can provide a diagnostic and/or therapeutic function. Accordingly, a cargo polypeptide can produce a detectable signal such as, for example, fluorescence, magnetic resonance, radioactivity, positron emission tomography, photoacoustic or other signal detectable with commercially-available equipment. Alternatively or additionally, a cargo polypeptide can provide therapeutic activity. As used herein, “therapeutic activity” refers to activity that ameliorates one or more symptoms or clinical signs associated with a condition and/or reduces the likelihood or extent to which a subject receiving the cargo polypeptide develops a condition compared to a subject who does not receive the cargo polypeptide. Accordingly, a cargo polypeptide can exhibit cytotoxicity. Exemplary therapeutic cargo proteins include, for example are the apoptotic proteins caspase 3, tumor necrosis factor alpha (TNA-alpha), apotin and p53. Other examples include immunotoxins such as dipthereria toxin, ricin or gelonin. Monoclonal antibodies might also be delivered via the HGN. Stem cell associated proteins such as Sox2 and Oct4 are also appropriate for delivery via the HGN carrier. Sox2 is a transcription factor (TF) [SRY(sex-determining region Y)-box2] protein that binds to the Cyclin D1 and Nanog promoters. Sox2 is differentially expressed in prostate cancer cell lines (e.g., BPH-1, LNCaP, 22RV1, and DU145). Sox2 overexpression results in lower levels of Orail, a protein associated with store-operated Ca²+entry (SOCE), which confers apoptotic resistance to prostate cancer cell lines.

Many genes are under the control of multiple transcription factors, and the ability to simultaneously regulate distinct genes and pathways in the same cell, or isolated cells in culture, has many basic research applications. Another example is the hexapeptide, L-Y/F-P-W-M-K/R (SEQ ID NO:2), which disrupts the normal function of the HOX protein by antagonizing its binding with its cofactor PBX, which is responsible for increasing DNA binding affinity in melanomas, malignant B-cells, prostate cancer cells, and primary non-small-cell lung cancers (NSCLCs). The targeted delivery system described herein can deliver this—or any other—therapeutic peptide into cells with spatio-temporal control and/or improved efficiency due, at least in part, to the multiple copies of the therapeutic peptide per carrier (e.g., HGN). The carrier carrying the hexapeptide can disrupt the PBX cofactor binding with the HOX protein after release, thereby altering gene expression including, for example, the up-regulating cFos, Jun, Dsup1, and/or Atf3, each of which can lower cell viability. Another exemplary transcription factor peptide is TLKIVW (SEQ ID NO:3), which is an amyloid plaque inhibitor.

Alternative cargo proteins can include, for example, a signal transducers and activator of transcription (STAT), many of which are well-characterized proteins shown to affect a variety of cellular functions by targeting gene expression and repression. For example, STAT1 negatively regulates angiogenesis by inhibiting the genes necessary for response to vascular endothelial growth factors (VEGF). Neu/Her-2 is a prominent proto-oncogene found in prostate cancers such as in DU145 and 22RV1 cell lines. Activation of STAT1 in these cells has led to significant reductions in neu/Her-2 expression resulting in antiproliferative activity, which is enhanced with the increased expression of p21, a growth inhibitor. Similar activity has been demonstrated in ovarian cancer cell lines and colorectal adenocarcinomas resulting in the induction of proapopotic pathways.

Other proteins that may be suitable cargo protein include, for example, a genome editing proteins such as Cas9 or the zinc-finger nucleases. Recent advances in the control of cellular transcription and protein expression have relied on light-responsive transcription factors, which act quickly, are minimally invasive, and can provide dose-dependent control over protein levels. For example, the CRISP/Cas9 enzyme can edit the genome at predetermined sites.

The cargo protein can include a targeting peptide. The targeting peptide can target binding and/or uptake of the cargo polypeptide to a target cell population or cellular compartment. While described above in the context of an embodiment in which the cargo polypeptide includes an RPARPAR (SEQ ID NO:1) sequence, which targets the cargo polypeptide to the model target cell PPC-1 prostate cancer epithelial cells, in alternative embodiments that targeting peptide can be any peptide that specifically targets delivery of the cargo polypeptide to a cell population and/or cellular compartment. As used herein, “target” used as a verb, “specifically targets,” and variations thereof refer to having a differential or a non-general affinity, to any degree, for a particular target cell or target cell compartment. Accordingly, in some embodiments, the targeting peptide can be any one of many cell-penetrating peptides (CPPs) such as, for example, iRGD, a protein transduction domain, or a protein that facilitates membrane poration or endocytotic delivery (e.g., the HIV-1 transactivator of transcription (TAT) peptide). In other examples, polyarginine can be used as a cell penetrating peptide.

In another aspect, this disclosure describes a system for the delivery of a cargo polynucleotide to cell. Since its discovery in 1998, RNA interference (RNAi) has been recognized for its potential to control the flow of genetic information. The routine use of RNAi for disease treatment or prevention still calls for novel methods of delivery with spatial and temporal control. Current techniques in nucleic acid delivery include viral vectors and lipid vesicles, which have enhanced both bio-stability and bio-availability. One challenge for processes involving RNAi using nonviral vectors is endosomal escape into the cytosol, which is necessary for gene silencing to occur. siRNA is typically prevented from reaching the site of action due to encapsulation in endosomes, and eventually the siRNA is vulnerable to degradation enzymes in late endosomes or expelled from the cell by exocytosis. The efficacy of siRNA delivery is largely governed by the ease and rapidity of the escape from endo-lysosomes.

The modular nanocarrier platform described above may be designed to provide an efficient approach to release a cargo polynucleotide (e.g., siRNA) to a cell. In particular, this disclosure describes delivery of a model therapeutic siRNA specifically to a model population of target cells. The polo-like kinase (PLK1), expressed by the plk1 gene in prostate cancer cells, was targeted for the delivery of a therapeutic siRNA to induce a pro-apoptotic pathway. RNAi-based plk1 gene down-regulation forms the basis of Phase I/II clinical trials for the treatment of solid tumors. Peptides can provide advantages over other targeting approaches (e.g., antibodies) due to their small size, synthetic versatility, and cell and tissue specificity.

This disclosure also describes delivery of a cargo siRNA via a versatile and modular nucleic acid architecture that does not require thiolated RNA, increasing the sequence space that may be delivered. These novel architectural features can promote biomedical use of nanoparticles and RNAi for spatially patterned or cell selective gene expression.

### Assembly and Characterization of the Delivery Vehicle

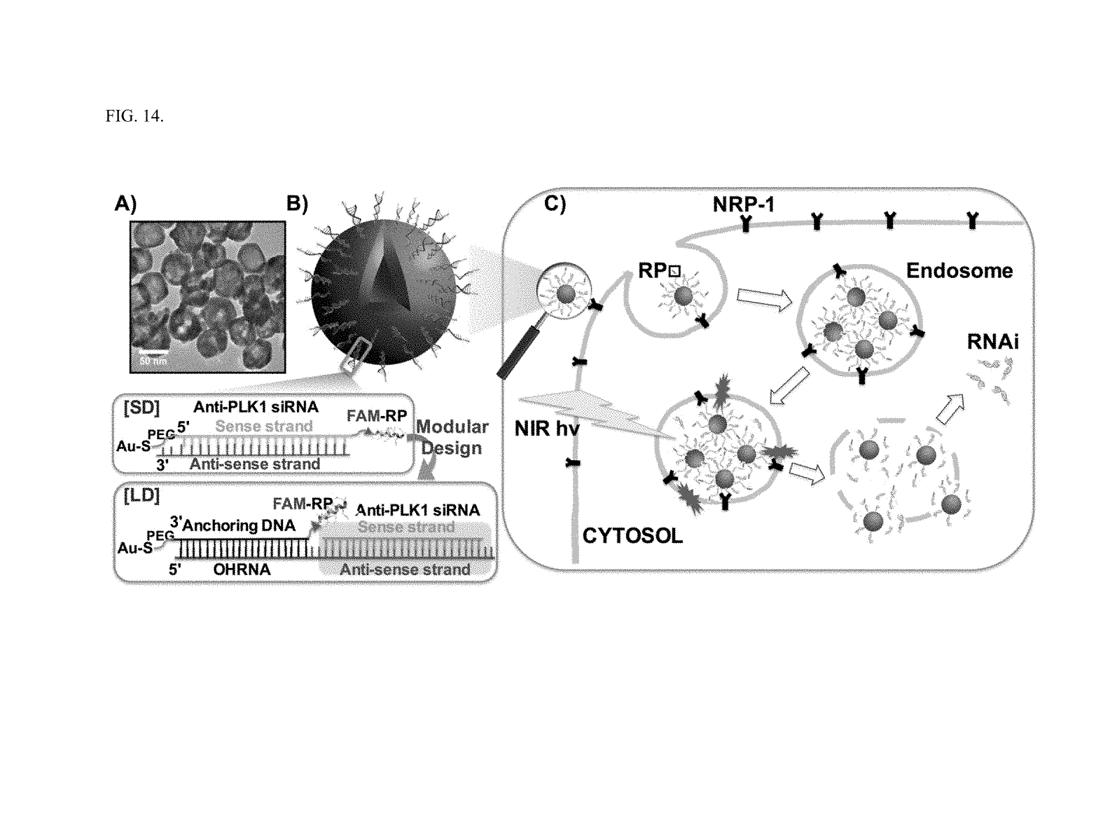

The modular nanocarrier platform described above can be expanded to include, as cargo, a variety of polynucleotides (e.g., RNA) (FIG. 14B). In the exemplary model embodiment illustrated in FIG. 14, multiple copies of siRNA against plk1 were conjugated to the surface of an approximately 50 nm HGN, either directly or supported by a single strand DNA linker, through a quasi-covalent (Au—S) bond. This design can increase drug content, stability, and/or the number of linkers available for multivalent presentation of peptides (FIG. 14B). The embodiment illustrated in FIG. 14 employs a peptide following the C-end rule (CendR), RPARPAR (RP, SEQ ID NO:1), that binds specifically to the neuropilin-1 (NRP-1) receptor that is over-expressed by certain types of cancer cells. The siRNA- (as a model cargo polynucleotide) and RP-coated HGNs internalize into endosomes (FIG. 14C). Upon pulsed NIR laser irradiation, the Au—S bond linking the siRNA to the HGN surface is ablated while the conversion of light energy to heat produces a transient vapor bubble that ruptures the endosome without damaging the siRNA or the cell, releasing the cargo into the cytosol (FIG. 14C). This combination of steps results in highly efficient transfer of cargo polynucleotide with specificity from the both the targeting peptide and the laser irradiation to ensure RNAi function occurs only in the doubly targeted cells.

As with the modular nanocarrier constructs described in detail above for the delivery of cargo polypeptides, modular nanocarrier constructs for the delivery of cargo polynucleotides can include any suitable carrier. Thus, while describe below in the context of an exemplary embodiment that includes a hollow gold nanoshell (HGN) as the carrier, in alternative embodiments the carrier may be any solid substrate to which the irradiation-cleavable linker may be attached including, for example, a CuS nanoparticle, a silver or gold nanocube, a silver or gold nanoshell, or a solid silver or gold nanoparticle. Thus, unless used in the context of describing a specific embodiment, reference to a hollow gold nanoshell or use of the HGN as an abbreviation therefore, is merely exemplary and used for brevity to describe what, in certain embodiments, can be a carrier that includes any of the suitable carriers listed in this paragraph. Similarly, unless used in the context of a specific exemplary embodiment, reference to a thio-gold bond is merely exemplary and is used for brevity to describe what, in certain embodiments, can be a thio-silver bond or thio-copper bond.

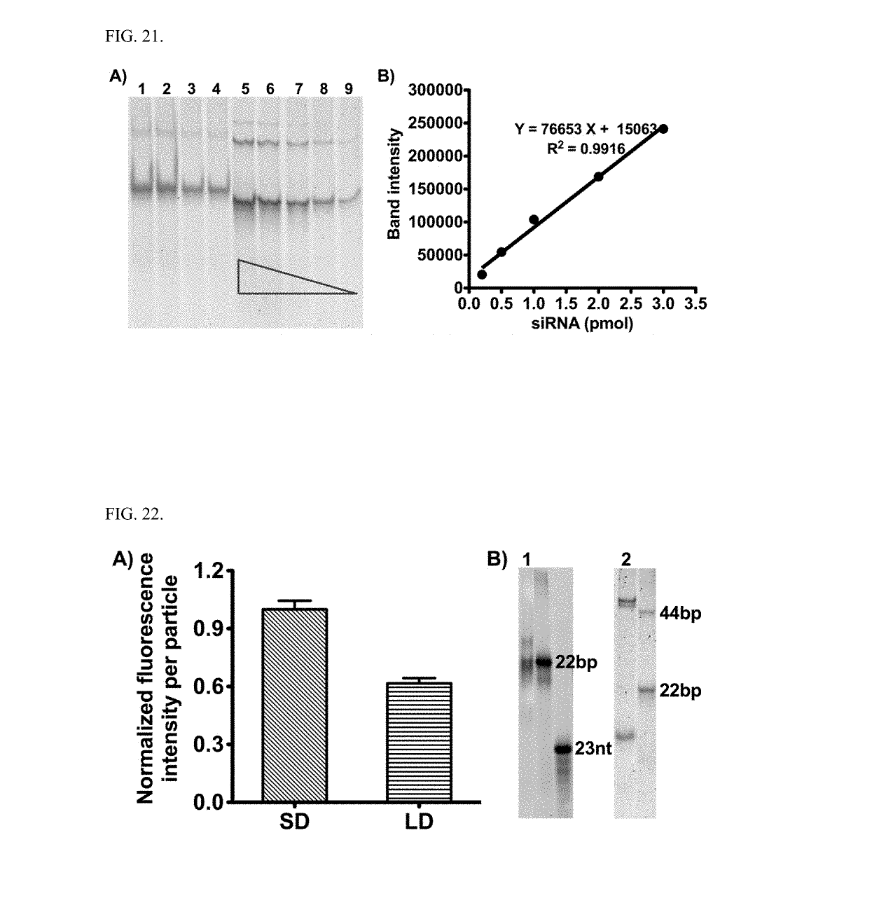

To prepare the model siRNA functionalized nanoparticles (HGN-SD-RP, FIG. 14B) for gene knockdown, siRNA sense strands containing 5′ thiol modifications were assembled onto the citrate passivated HGN surface using a low pH-induced self-assembly method as previously described (Zhang et al., 2012, 1 Am. Chem. Soc. 134(17):7266-7269). RNA strands were added to HGNs in low pH (3.0) sodium citrate buffer to neutralize the negatively charged phosphate backbone and maximize the assembly rate. Anti-sense RNA strands were then hybridized to the sense RNAs, resulting in approximately 2300±600 siRNA duplexes per particle (FIG. 20A, FIG. 21). The residual HGN surface was then passivated with thiol-polyethylene glycol (PEG)-amine (3 kDa) and 6-mercapto-1-hexanol (MCH). The RP peptide, carrying a fluorescein dye (FAM) for tracking and characterization as well as a cysteine for conjugation, was incorporated to the HGN-siRNA via short PEG linkers to bridge the amine on the 3′ sense strand and the thiol (cysteine) on the peptide to enable cancer cell-specific targeting (FIG. 20A). Initially the citrate HGN showed a resonance at ˜710 nm, which red-shifted to ˜810 nm as the layers were assembled (FIG. 20C).

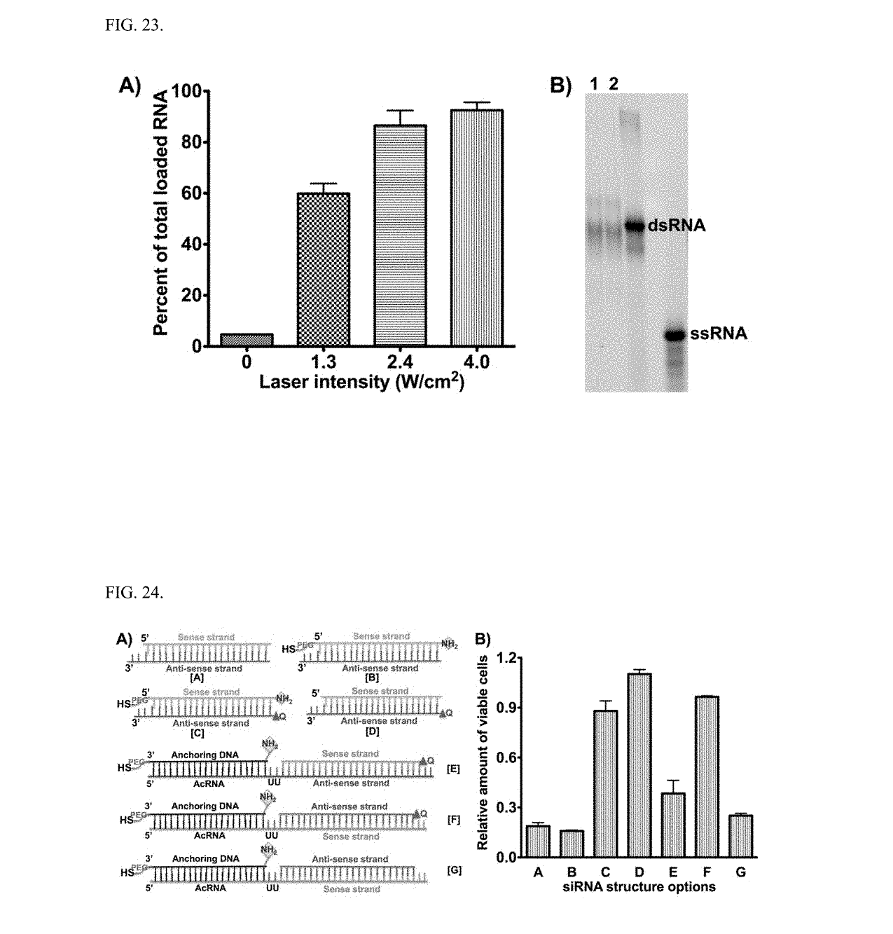

Irradiation of the final product HGN-SD-RP with pulsed laser light (1 kHz repetition rate, ˜120 fs pulse length) at 800 nm at a laser power density of 2.4 W/cm² for 10 s released ˜85% of total siRNA (FIG. 23A, 100% release achieved by HGN dissolution using KCN). The absorbed energy caused the Au—S bonds to be cleaved, but the siRNA remained hybridized and biochemically active after the laser release (FIG. 23B).

Endosomal Escape of siRNA

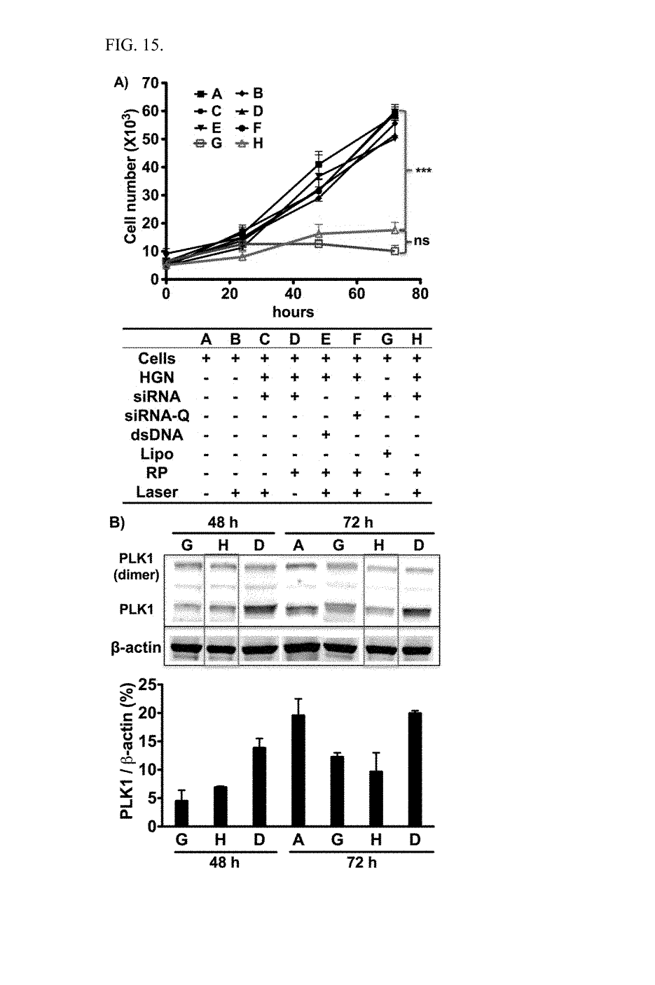

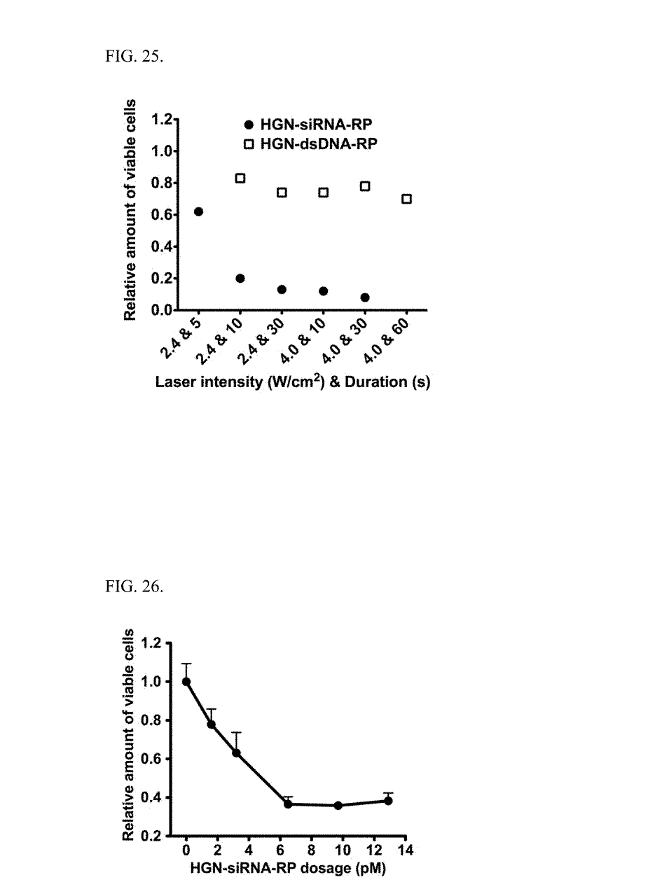

The down-regulation effect of plk1-siRNA released from HGNs was tested using the epithelial prostate cancer cell line PPC-1, which overexpresses the NRP-1 receptor targeted by the RP peptide. Western blot and cell viability assays were used to assess and quantify down-regulation of plk1 (FIG. 15). Irradiation with the femtosecond pulsed NIR laser (2.4 W/cm² for 10 s) of cells exposed to HGN-SD-RP led to 70% loss of cell viability at 72 hours (FIG. 15A, sample H), similar to the effect of unconjugated siRNA transfected using commercial LIPOFECTAMINE RNAiMAX (Lipo; Life Technologies, Thermo Fisher Scientific, Grand Island, N.Y.). The LIPOFECTAMINE treatment required, however, approximately 13-fold more siRNA (0.37 nM siRNA compared with 5 nM siRNA for 5000 cells) (FIG. 15A, sample G). Moreover, like other nanoparticle approaches that also require considerably more siRNA, LIPOFECTAMINE treatment lacks an efficient escape mechanism from endosomes. Cells exposed to the same laser conditions in the absence of HGN-SD-RP showed no loss of viability (FIG. 15A, sample B). HGN-siRNA-Q-RP (where Q indicates Quasar570 dye, placed here on 5′ anti-sense RNA, which blocked siRNA activity against plk1, FIG. 24) and HGN-dsDNA25 bp-RP (loaded non-functional dsDNA in place of the siRNA) were employed as negative controls and were also used to test whether the local heating caused by NIR laser irradiation in the presence of HGNs, induced cellular damage (FIG. 15A, samples E-F). The targeting peptide was necessary for the internalization of nanoparticles into PPC-1 cells, as laser treatment of cells exposed to HGN-siRNA without RP did not show any knockdown or cell death (FIG. 15A, sample C).

FIG. 15A shows that the HGN-SD-RP (sample H) was the only construct to cause significant loss of cell viability upon laser exposure. Cells exposed to HGN-SD-RP, but not to the NIR laser (sample D) showed no effect, demonstrating that laser irradiation was required to activate the biological response in the cells, providing for both spatial and temporal control of knockdown. The minimum laser exposure power and time for effective siRNA release in cells was 2.4 W/cm² for 10 s (FIG. 25). Western blot analysis confirmed the down-regulated plk1 expression level in PPC-1 cells treated with HGN-SD-RP and NIR laser after 48 hours and 72 hours. In FIG. 15B, lanes G and H exhibited an approximately 60%-70% decrease of the PLK1/β-actin band intensity compared to untreated cells (lane A) and to cells carrying HGN-SD-RP, but not irradiated by the laser (lane D). This result likely underestimated the knockdown, as only live cells were used for the Western analysis.

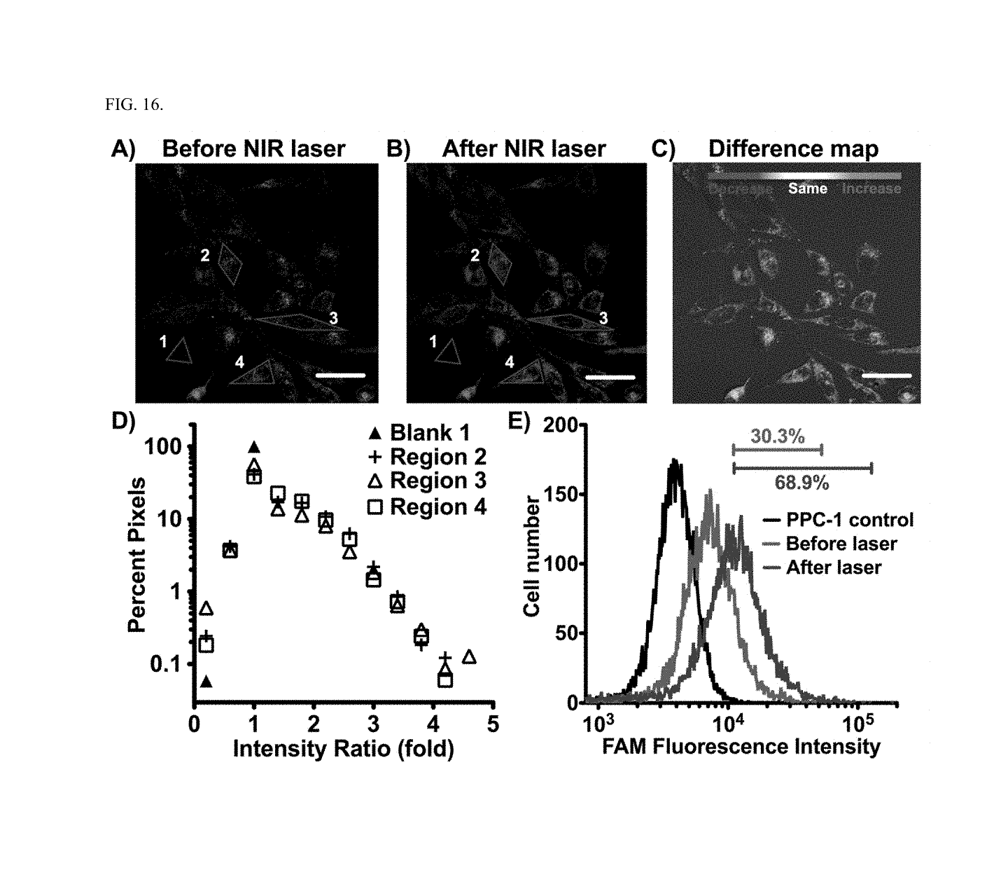

FAM-labeled HGN-SD-RP was readily internalized into PPC-1 cells (FIG. 16A). The siRNA release was assessed using both fluorescence confocal microscopy and flow cytometry. The FAM label on the HGN-SD-RP is approximately 50% quenched at distances of approximately 10 nm or less from the gold. When the SD-RP is released by the laser, the fluorescence roughly doubles in intensity. Femtosecond pulsed laser irradiation caused both a significant increase and expansion of FAM fluorescence (from individual puncta to more uniform, diffuse) in the cytosol of each cell, indicative of the release of siRNA-RP in individual cells as shown by confocal microscopy (FIG. 16A-C). Regions of interest were selected, enclosing either single cells (Regions 2, 3, and 4 in FIG. 16A-B) or the glass slide background (Region Blank 1) to conduct pixel intensity analysis (FIG. 16D). 40-60% of the pixels in cells showed greater than 1-fold increase in intensity, whereas almost 100% of the pixels in the Blank 1 region showed no fluorescence increase (Ratio ˜1) following laser treatment. Flow cytometry showed an increase in average intensity (FIG. 16E). Approximately 30% of the PPC-1 cell population after HGN-SD-RP internalization showed a significant fluorescence signal, defined by being above the brightest 1% of the unlabeled control cells. The percentage increased to ˜70% after laser irradiation (FIG. 16E), due to a 2.5-fold increase in mean cell intensity that was consistent with maximal release of the SD-RP from the HGN. This also suggests that at least 70% of the cells efficiently internalized HGN-SD-RP.

The minimum HGN-SD-RP dosage used for efficient siRNA knockdown and cell death (FIG. 26) was 6.5 pM nanoparticles carrying 15 nM siRNA for 2×10⁵ cells (approximately 4000 nanoparticles per cell). The reported concentration represented the amount of HGN-SD-RP available per cell; the amount internalized was likely lower. Even so, the concentration was orders of magnitude less than other nanoparticle approaches that required 10⁶-10′ nanoparticles or more per cell to get effective knockdown of the respective genes (Zheng et al., 2012, Proc. Natl. Acad. Sci. U.S.A 109(30):11975-11980; Zhang et al., 2012, J. Am. Chem. Soc. 134:(40), 16488-16491; Lu et al., 2010, Cancer Res. 70:(8), 3177-3188). Meanwhile, the plk1-siRNA dosage needed for maximum cell viability loss by this method was approximately one-tenth that of other reported plk1-siRNA delivery methods (Dassie et al., 2009, Nat. Biotechnol. 27(9):839-849; Yang et al., 2012, ACS Nano 6(6):4955-4965; Zhou et al., 2012, Biomaterials 33(2):583-591; Gu et al., 2013, Biomacromolecules 14(10):3386-3389; Yang et al., 2011, J. Control. Release 156(2):203-211; Xiang et al., 2013, Biomaterials 34(28):6976-6991). Lower particle and siRNA concentrations may result from efficient particle internalization and/or efficient siRNA escape from the endosomes. Particle internalization efficiency may be the result of peptide targeting and endocytosis. Efficient siRNA escape may result from vapor bubble formation and endosome rupture.

### Prostate Cancer Cell-Specific Targeting

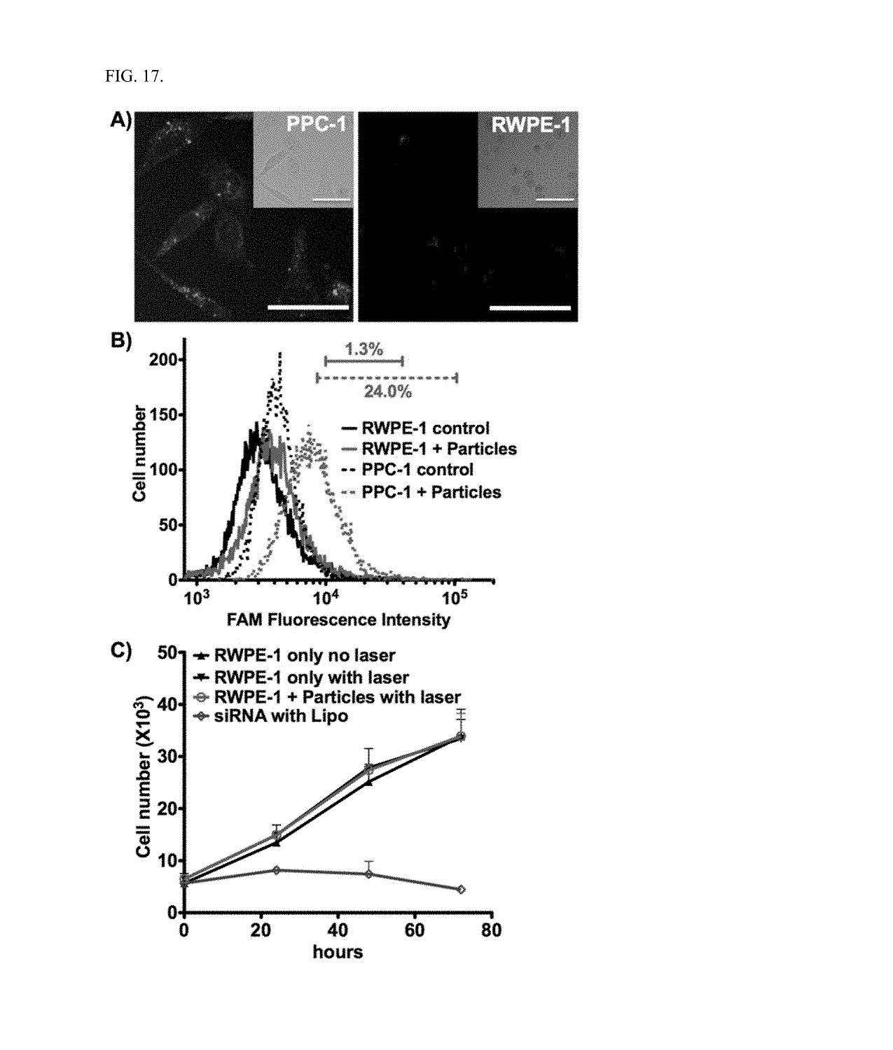

Normal human prostate epithelial RWPE-1 cells lack the NRP-1 receptor on the cell surface, resulting in negligible HGN-SD-RP internalization (FIG. 17A). By flow cytometry, only about 1% RWPE-1 cells were above threshold fluorescence intensity, compared to 30% of PPC-1, which express NRP-1 (FIG. 17B). Thus, there was no down-regulation of plk1 or loss of cell viability on laser treatment of RWPE-1 cells (FIG. 17C). However, RWPE-1 cells were sensitive to plk1-siRNA, as shown using the non-selective LIPOFECTAMINE transfection of the siRNA construct (FIG. 17C, siRNA with Lipo).

### Modular RNA Assembly

The exemplary HGN-SD-RP embodiment illustrated in FIG. 14B involves a thiolated RNA to attach to the SD-RP to the HGN, similar to other nanoparticle-based approaches. After validating the release efficiency, the targeting peptide specificity, and the siRNA function in knocking down plk1 expression, we created a new tethering molecule that could integrate all the above functionalities into a versatile modular architecture with greater flexibility for delivering an generic siRNA cargo (HGN-LD-RP, FIG. 14B), with the possibility of alternative peptide targeting sequences. Thus, this disclosure describes a universal HGN-DNA assembly requiring only the addition of non-modified (e.g., less expensive and more readily available) RNA. An anchoring thiol-DNA-amine strand was assembled on the HGN and later conjugated to the targeting peptide, as a replacement for the anchoring by thiol-RNA-amine. This core module was hybridized to a siRNA precursor designed with an overhang on its anti-sense strand complimentary to the anchor sequence (OHRNA) (FIG. 14B). A variety of siRNA orientations and dye labels and positions were tested, keeping a constant OHRNA sequence (FIG. 24A, structures E-G).

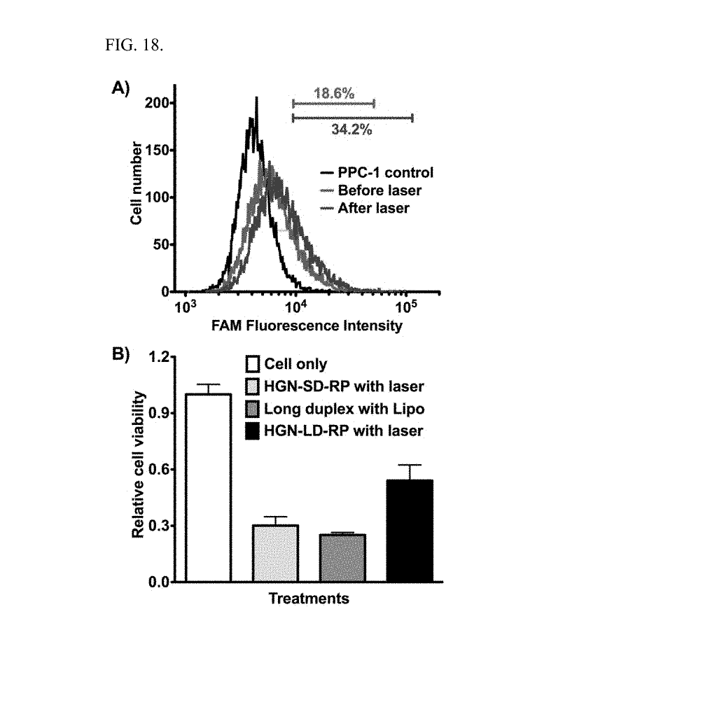

The gene knockdown activity of the various combinations were compared to conventional LIPOFECTAMINE transfections. The 50 bp DNA-RNA ‘long’ duplex (LD) was nearly as effective as the short siRNA (FIG. 24B). Fluorescence-based quantification after KCN release of HGN-LD-RP showed that the number of larger LD strands per particle was ˜60% of the number of the smaller HGN-SD-RP strands (FIG. 22). The down-regulation activity of the HGN-LD-RP was assessed on PPC-1 cells as with the HGN-SD-RP siRNA system. Cell exposure to the laser at 2.4 W/cm² for 10 s resulted in 46% reduction of cell viability at 72 hours, in comparison with 70% from the HGN-SD-RP, and from the same LD construct transfected by LIPOFECTAMINE (FIG. 18B).

Cell uptake efficiency and cargo release were also evaluated by flow cytometry. Approximately 19% of the PPC-1 cells incubated with HGN-LD-RP show fluorescence intensity above threshold before laser irradiation. The percentage increased to approximately 34% after laser treatment (FIG. 18A). One can further increase the efficiency of cell uptake by modifying the peptide conjugation strategy, optimizing the overhang sequence, and/or tuning the hybridization conditions to increase the cargo density.

In summary, this disclosure describes two illustrative modular plasmonic siRNA nanocarriers coupled to hollow gold nanoshells for RNAi-mediated gene knockdown. The exemplified construct was engineered to specifically target cancer cells using the RPARPAR (SEQ ID NO:1) ligand against over-expressed NRP-1 receptor on the PPC-1 prostate cancer cell surface to promote cell internalization via endocytosis. The results observed with the model construct demonstrate, however, that the constructs may be designed for use against, and the methods may be practiced to target, any suitable target cell.

The siRNA is released from the carrier and the endosome by femtosecond pulses of near infrared light at 800 nm: the light energy is converted into heat, which thermalizes the thiol bonds holding the siRNA to the carrier, followed by vapor bubble formation that ruptures the endosomes, without damaging the siRNA or the cell. This combination can provide cargo delivery with cellular level resolution at the desired time, using less—e.g., an order of magnitude less—siRNA than conventional techniques with comparable transfection efficiency. The results show minimum off-target toxicity as evidenced by the lack of cell viability changes with RWPE-1, which does not express NRP-1. Control of laser irradiation provides an additional targeting effect, as PPC-1 cells were also unaffected in the absence of laser treatment. The approach shows high selectivity and RNAi delivery with high efficiency, versatility and reduced cost. The modular design described in this disclosure provides a basis for alternative applications requiring only the annealing of unmodified siRNA precursor to previously prepared and generic (e.g., HGN-DNA) carriers. The combination of NIR laser-based release and endosomal escape, targeting peptide induced cell-specific internalization, and a versatile siRNA loading strategy allows one to use nanoparticles to target RNAi to specific cell types and even individual cells and furthers the possibility of using modular constructs for RNAi screening assays and for in vivo cancer therapy.

One application of the modular nanocarrier platform described above involves the delivery of a polynucleotide to a human embryonic stem cell (hESC). The capability of human embryonic stem cells differentiation into all types of cells in the body holds immense promise in tissue engineering and regenerative medicine. The modular nanocarrier platform described herein can be used to design a light-activated hESC silencing system.

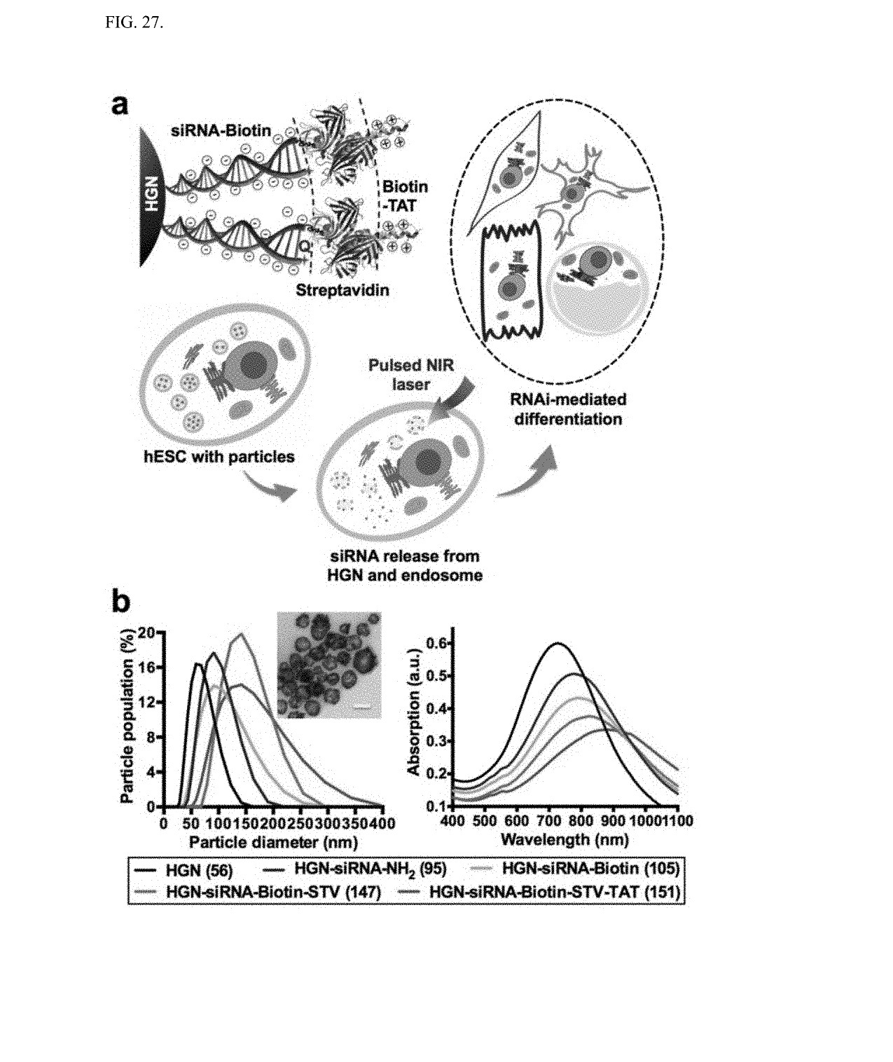

To create the hESC silencing system using the modular nanocarrier platform, siRNA molecules were densely assembled onto plasmonic hollow gold nanoshells (HGN) via thiol bond formation, then were overcoated with a protective protein layer with handles for attaching cell penetrating peptides. Upon irradiation with biocompatible near infrared (NIR) light (˜800 nm), the siRNAs were released from the gold surface by dissociation of thiol-gold bond at the carrier surface. Endosomal rupture resulted from vapor layer formation around the hot gold nanoparticles. In this work, we explored the ability of HIV-derived cell penetrating peptide (CPP) TAT (YGRKKRRQRRR, SEQ ID NO: 4) to facilitate the internalization of the HGNs into hESC. However, direct conjugation of TAT-peptide and siRNA-conjugated HGNs resulted in aggregation, presumably due to colloidal surface charge neutralization and bridging between the cationic TAT and the anionic siRNA. Thus, an alternative surface coating strategy was devised that positioned TAT on the siRNA through coupling with the tetravalent protein streptavidin, which sterically prevents the siRNA from electrostatic contacts and thus inhibits particle aggregation. The resulting construct (FIG. 27a) was capable of releasing siRNA payloads upon NIR laser irradiation and could efficiently internalize into a variety of hESC cell lines.

To validate the use of the modular nanocarrier platform in hESCs, GFP was silenced in engineered H9 cells, and Oct4 was silenced in the original H9 cells.

### Construction and Characterization of TAT Peptide Coated HGN-siRNA

Efficient delivery of siRNA to hESC is known to be difficult for synthetic vectors. To overcome this limitation, a multi-functional nanoparticle carrier was constructed by modifying the generic cell penetrating peptide TAT (YGRKKRRQRRR, SEQ ID NO:4) to have an N-terminal biotin, then attaching the modified TAT peptide onto hollow gold nanoshells (HGNs) functionalized with multivalent siRNA (FIG. 27a). Multiple copies of functional siRNA molecules were conjugated to the surface of the approximately 50 nm diameter HGN through a quasi-covalent Au—S bond. Uptake of the construct by hESC was mediated by the TAT-peptide coating on the particle surface. Without wishing to be bound by any particular theory of operation, pulsed NIR laser irradiation causes the HGNs to strongly absorb the pulsed NIR laser light, which is rapidly (nanosecond) converted to heat, ablating the Au—S bond holding the siRNA on HGN surface and producing transient vapor bubbles that rupture the endosome without damaging the siRNA or the hESC (FIG. 27a). This strategy results in the successful intracellular transfer of negatively charged siRNA to the cytosol of hESC, thereby initiating RNAi-mediated gene knockdown and stem cell differentiation.

Initial designs were unsuccessful. A TAT peptide was modified with a lipid to form a lipid bilayer around the HGN-siRNA, insulating against nanoparticle-nanoparticle electrostatic coupling. This strategy generated TAT-coated HGN in a stable colloidal formulation and efficient siRNA loading, but internalization in hESC was not sufficient to cause effective gene silencing upon laser treatment. Then, thiolated siRNA molecules were backfilled onto thiol-PEG5k-TAT coated HGN. The nanoparticles did not aggregate after siRNA backfilling and were internalized into hESC, but this approach showed inefficient laser knockdown of GFP in cells.

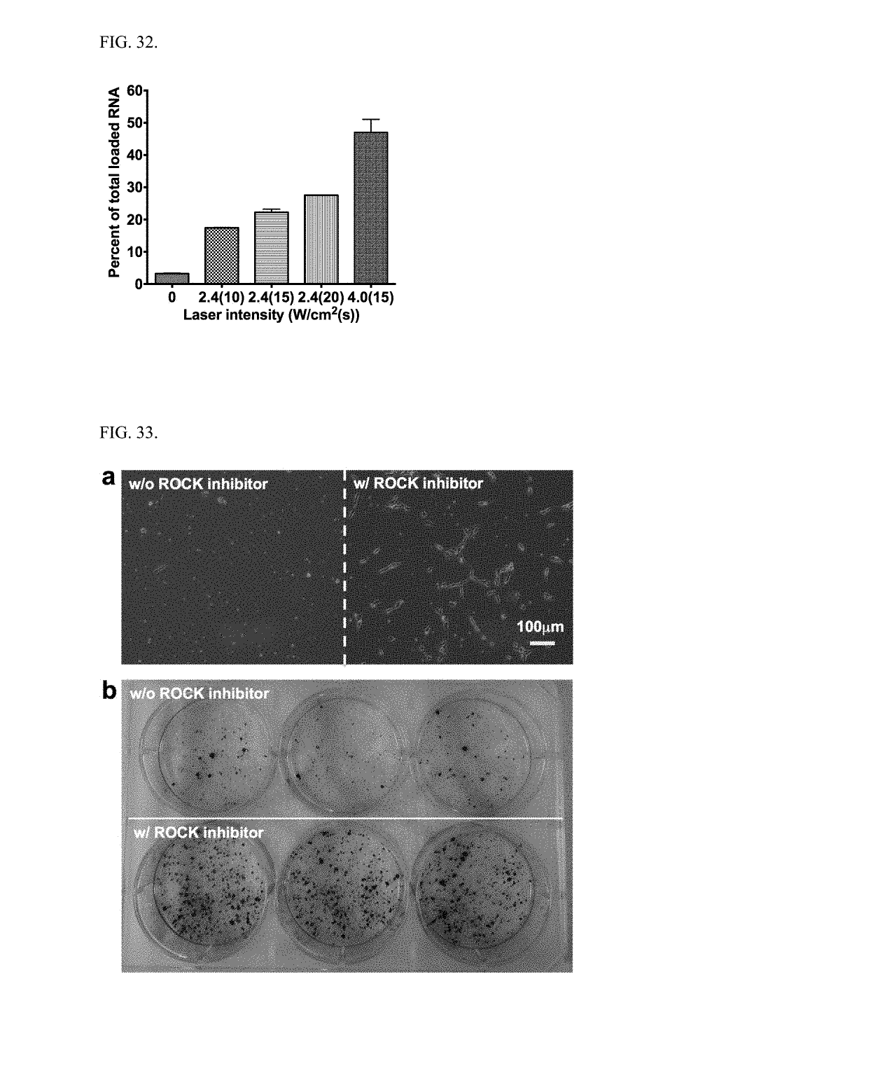

Instead, a high number of siRNA duplexes (approximately 2300) were conjugated to the surface of each HGN (approximately 50 nm in diameter) through low pH-induced self-assembly of thiolated RNA sense strand followed by the hybridization of the anti-sense strand as described previously (Huang et al., 2014, Nano Lett. 14(4):2046-2051). An approximately 5 nm streptavidin bridging element was placed between a biotinylated RNA layer and a biotinylated TAT-peptide (FIG. 27a). The average hydrodynamic diameter of the final construct HGN-siRNA-Biotin-STV-TAT (HRT) increased from 56 nm for bare HGN (citrate stabilized) to 151 nm (FIG. 27b), which can be attributed to the sum of RNA length, protein/peptide coating, and slight aggregation. The plasmon resonance of the nanoparticles red-shifted from ˜710 nm for bare HGN to ˜880 nm after the final coating step (FIG. 27b). Irradiation of HRT by a pulsed NIR laser at 800 nm wavelength caused the release of ˜530 siRNA duplexes per particle (˜23% of capacity), at a power density of 2.4 W/cm² for 15 s (FIG. 32) with 1 kHz pulse repetition rate, ˜120 fs pulse duration. This streptavidin-TAT coating strategy was sufficient to cause the desired biological effect.

HGN-Mediated GFP Knockdown Evaluation in Transduced hESC

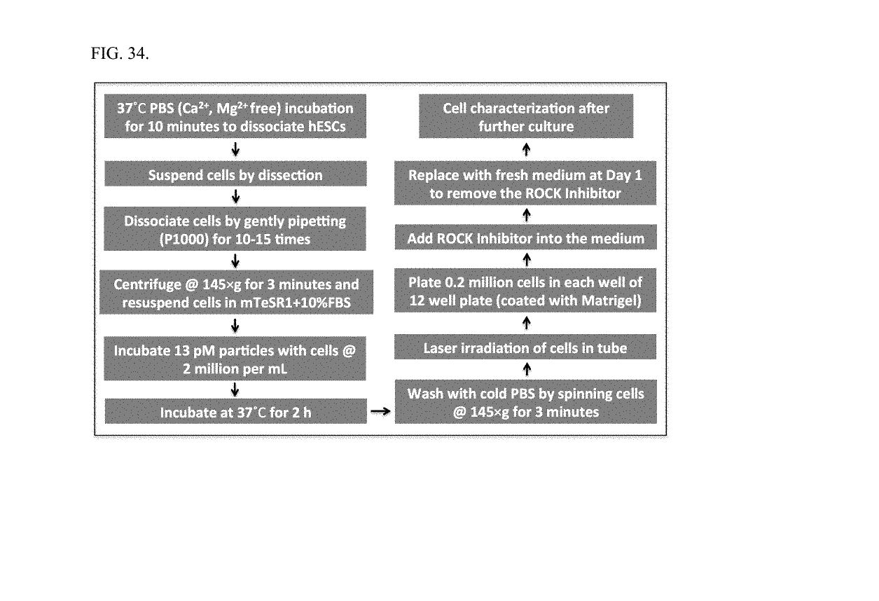

The compact clusters of hESC reduce accessibility to transfection. To maintain cell viability, a non-enzymatic method for dissociating the hESC was used. The method generated single cells and small cell aggregates (5-10 cells) while causing minimum cell damage. Briefly, a 10-minute incubation of attached hESC with Ca²⁺ and Mg²⁺ free PBS (37° C.), followed by manual dissection was sufficient for cell dissociation. Cells generated by this mild treatment appeared to take up more nanoparticles than cells obtained using commercial non-enzymatic cell dissociation buffer treatment. A rho-associated protein kinase (ROCK) inhibitor within the first 24 hours after single cell seeding significantly increased the cell viability (FIG. 33). An exemplary HGN transfection protocol (FIG. 34) enables both efficient cellular uptake and robust cell viability.

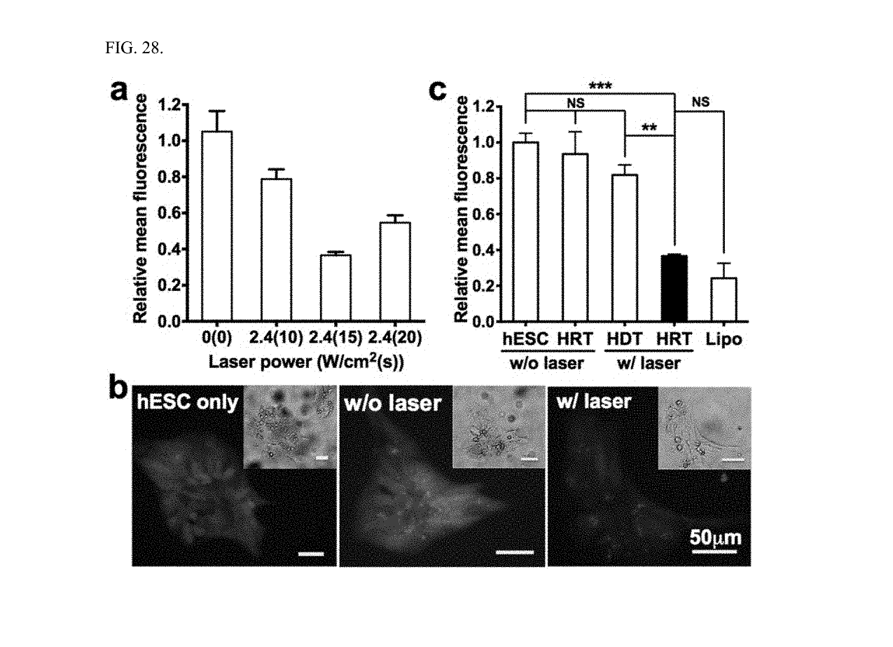

To demonstrate the NIR laser-dependent siRNA activation in hESC, HGN particles carrying GFP-siRNA were incubated with GFP-expressing hESC (H9-GFP), followed by NIR laser irradiation. Nanoparticles with 6 pmol siRNA were used for 4×10⁵ cells (˜4000 nanoparticles per cell). A set of different laser powers was tested, and 2.4 W/cm² for 15 seconds was found to have the maximum GFP silencing effect (FIG. 28a) and was selected for later studies. Fluorescence imaging of H9-GFP cells 3 days post treatment showed that GFP expression in cells decreased significantly only when cells carrying particles were irradiated with laser (FIG. 28b).

Flow cytometry quantification of GFP fluorescence in H9-GFP cells at day 3 showed that the mean fluorescence of the whole cell population after particle internalization and laser treatment decreased by ˜60% compared to cells without particle and laser treatment (FIG. 28c). This down-regulation efficiency is similar to the best data using commercially available transfection reagents including LIPOFECTAMINE RNAiMAX (Life Technologies, Thermo Fisher Scientific, Grand Island, N.Y.) (FIG. 28, Lipo), but requires approximately one-tenth of the siRNA that the commercial reagents require.

The knockdown efficiency of the method using the modular nanocarrier platform can be further increased by, for example, optimizing particles to release more of the siRNA load upon laser treatment and/or by modifying the laser irradiation protocol to illuminate the entire cell population. GFP expression remained unchanged in the controls, including cells incubated with GFP-siRNA-carrying HRT without laser irradiation, cells incubated with non-sense dsDNA-carrying HDT and with laser irradiation, and cells without nanoparticles or laser treatment (FIG. 28c). Thus, down-regulation of GFP results from siRNA being released from HRT nanoparticles, and that NIR laser irradiation is necessary for the siRNA-mediated GFP silencing in the cells.

Nanoparticle Internalization in Un-Engineered hESC and Analysis of Cytotoxicity

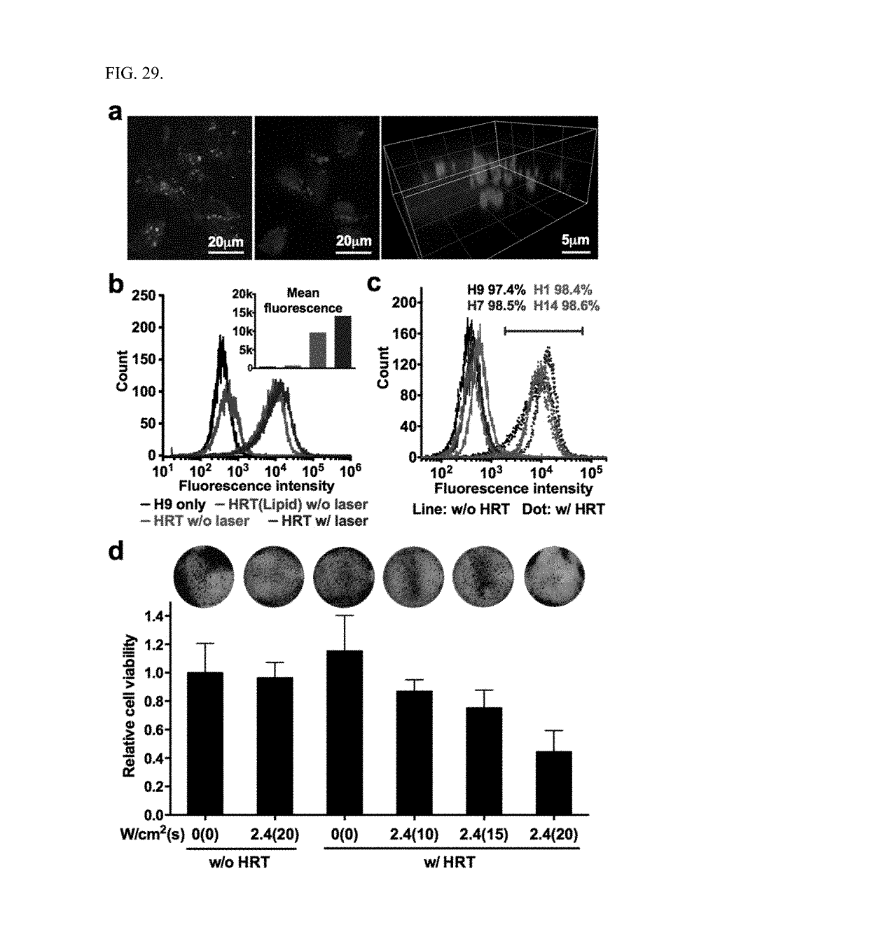

The HRT nanoparticle was then tested for the penetration into un-engineered hESC with the protocol illustrated in FIG. 34. siRNA was labeled with Quasar570 to track the internalization of HRT into hESC H9 cells over a two-hour incubation at 37° C. Dark-field microscopy of cells after nanoparticle incubation showed orange-red puncta surrounding the cell nucleus and this co-localized with the red fluorescence puncta from Quasar570 (FIG. 29a). Confocal fluorescence microscopy 3D images of selected single cells confirmed the intracellular distribution of the siRNA (likely inside endosomes) near the nucleus (FIG. 29a). Cellular internalization of HRT was quantified by flow cytometry using the Quasar570 fluorescence intensity and the fluorescence change after pulsed NIR laser irradiation (FIG. 29b). Approximately 97% of the H9 cell population showed significant fluorescence due to HRT uptake, defined as being brighter than the brightest 1% of the unlabeled control cells. Moreover, the mean fluorescence of the cells was increased by 44% after laser irradiation, consistent with RNA payload release (since Quasar570 is partially quenched when near the gold surface). Importantly, HRT showed similar high internalization efficiency (>97%) across a series of hESC cell lines including H1, H7, and H14 (FIG. 29c).

To assess any effect from particles and laser treatment to cell viability, H9 cells containing HRT with double-stranded RNA (non-sense to H9 cells) were exposed to NIR laser of different powers, and cultured on MATRIGEL-coated plates for five days. Live cells growing on the plate were stained with crystal violet, while dead cells that had lost the ability to attach were washed during fresh medium exchange every other day. FIG. 29d shows that the laser irradiation with power and time at or below 2.4 W/cm² for 15 seconds had no significant impairment on cell viability, compared to untreated control cells. Cell stemness was also not affected, judging by cell morphology seven days after particle and laser treatment (2.4 W/cm² for 15 seconds), since it was normal for untreated cells to have large nuclei and be tightly packed to form colonies with smooth edges. Moreover, since the expression levels across a panel of stem cell biomarkers was not changed after laser irradiation of cells loaded with HDT, which carries a control double-strand DNA (non-sense to H9), specific stemness changes can only be related to the RNA activity, not the effects of the laser, gold, streptavidin, or peptide.

Light-Activated Oct4 Knockdown in hESC Accelerates Stem Cell Differentiation

To demonstrate delivery of siRNA to the original un-engineered H9 cells and down-regulate a gene with biological activity, Oct4, which is a transcription factor for embryonic stem cell self-renewal and pluripotency, was selected as a model target. The down-regulation of the protein encoded by Oct4 initiates and accelerates differentiation.

siRNA knockdown of the Oct4 gene in H9 cells was first tested using a lentiviral transfection method with the plasmid LL-OCT4-1/2 (Addgene, Cambridge, Mass.). The down-regulation of Oct4 expression in hESC H9 cells using this system initiated the differentiation process in the mTeSR1 medium (control was only barely differentiated) and accelerated this process in the differentiation medium (control was slower). The cells stained positive for all three germ layer markers after extended culture in the differentiation medium.

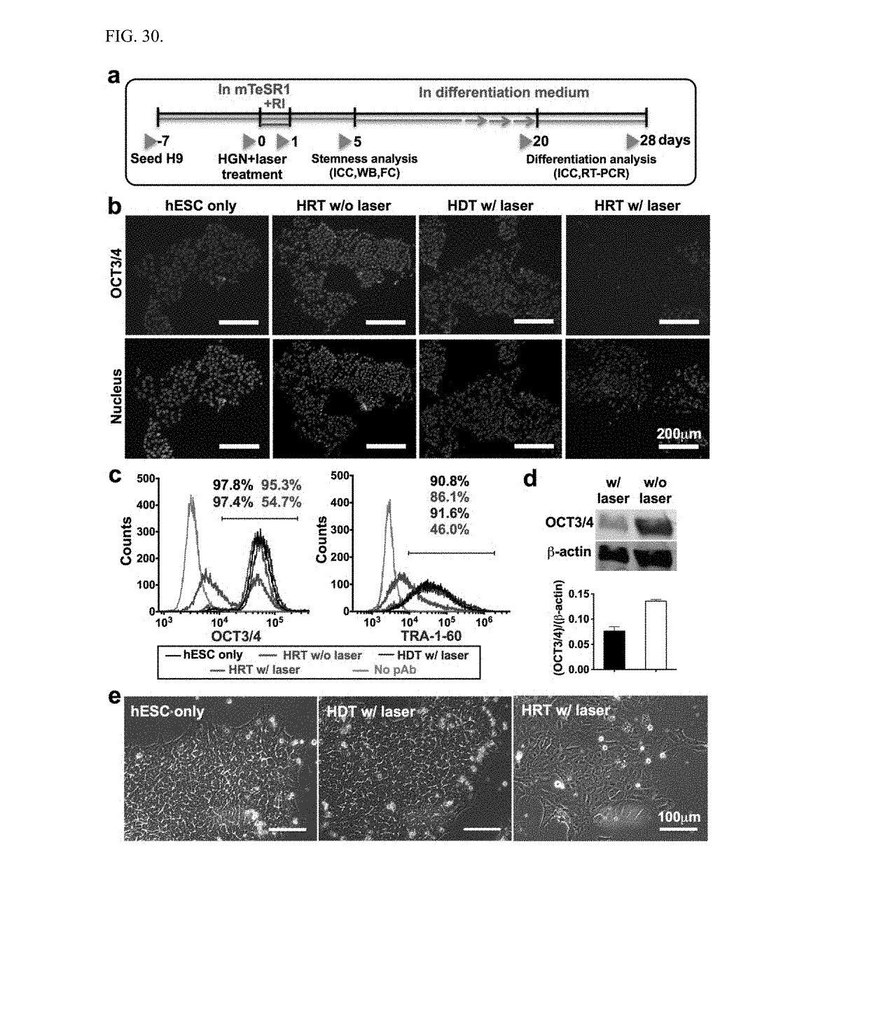

In parallel, H9 cells were incubated with the HRT construct HGN-siRNA(Oct4)-TAT and treated with the NIR pulsed laser at 2.4 W/cm² for 15 seconds, followed by cell assays after being cultured for five days in mTeSR1 medium (FIG. 30a). These conditions stimulated Oct4-siRNA function, as demonstrated by a significant decrease of OCT4 protein expression level at day 5, confirmed by immunocytochemistry (ICC) staining and Western blot assay (FIG. 30b-d). Flow cytometry quantification at day 5 (cells cultured in mTeSR1 medium) showed that approximately 50% of the cell population had decreased their expression of stem cell markers including OCT3/4 and TRA-1-60, as evidence of cell differentiation (FIG. 30c). Limited by cell number, our analysis of the knockdown and differentiation assay carried on day 5 might be an underestimation of the true knockdown efficiency since we noticed that undifferentiated cells divide more quickly than differentiated cells in mTeSR1 medium.

FIG. 30e shows that cells cultured with HRT and exposed to laser treatment exhibited differentiated cell characteristics: enlarged cell size, increased cytoplasmic area with decreased nuclear-to-cytoplasmic ratio. Consistent with the GFP knockdown experiments, laser exposure of HRT was required for the knockdown effect. Cells treated with HDT (HGN-dsDNA-TAT carrying non-sense dsDNA) did not show any apparent down-regulation of OCT4 protein, loss of stem cell markers, or stem cell morphology change (FIG. 30c and FIG. 30e), supporting that the particle internalization and laser treatment themselves have no side effects on cell stemness. Together, the results confirm that the down-regulation of Oct4 gene in H9 cells by this strategy leads to stem cell differentiation in a NIR laser-dependent manner.

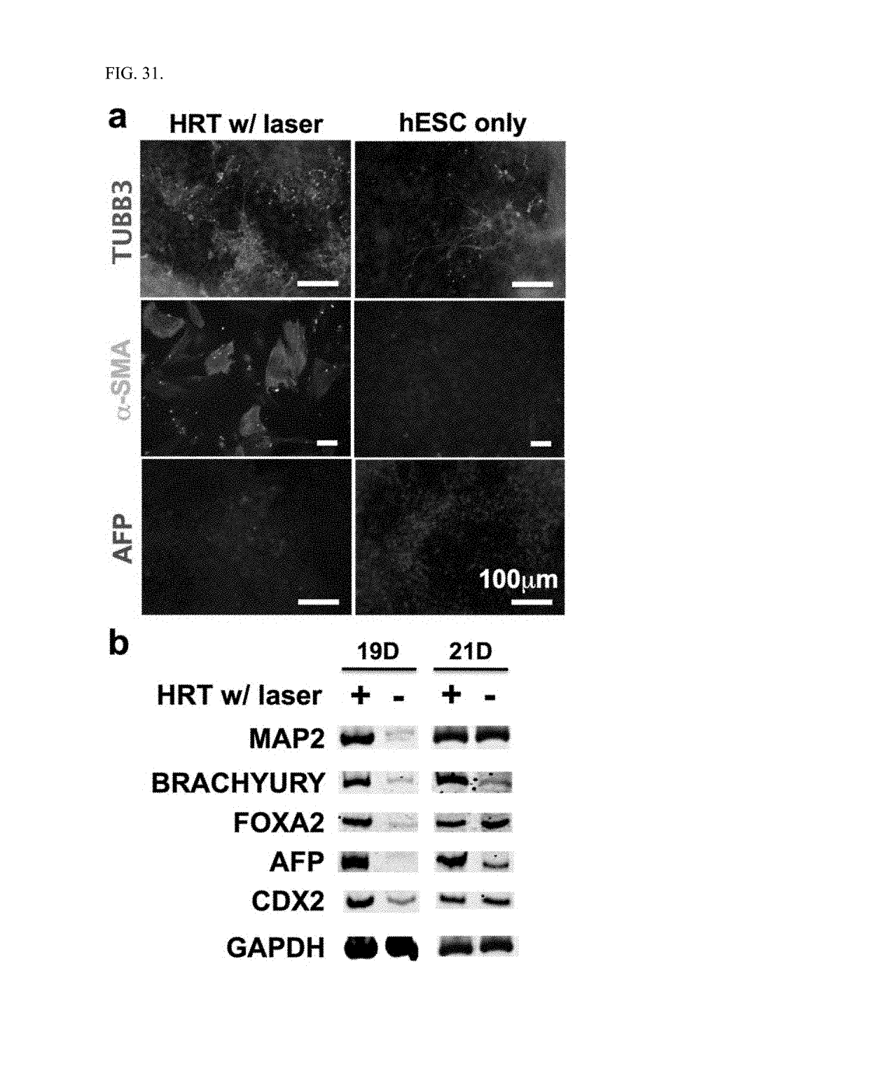

As laser-induced siRNA delivery in hESC has not been explored previously, the possibility that this method might cause cell differentiation to be biased toward certain germ layers was investigated. Cells were siRNA-treated as illustrated in FIG. 30a and then assayed from day 20-28 by ICC staining of three germ layer biomarkers including βIII-tubulin (TUBB3, for ectoderm), α-smooth muscle actin (α-SMA, for mesoderm), and α-fetoprotein (AFP, for endoderm), and reverse transcription PCR (RT-PCR) analysis of several biomarkers' mRNA expression. After the particle and laser treatment, the cells were switched to the differentiation medium to avoid the overwhelming growth of undifferentiated cells in mTeSR1 medium, then further cultured and assayed. Consistent with our observation from lentiviral Oct4 knockdown, HGN- and laser-dependent Oct4 knockdown accelerated the differentiation process compared to untreated self-differentiation in differentiation medium, as indicated by biomarker ICC staining and RT-PCR analysis (FIG. 31). H9 cells without particle and laser treatment (but with the same culture protocol) showed a delay, with α-SMA expression at day 28 (FIG. 31a), and apparently lower expression of TUBB3 and AFP at day 28, as compared to cells with particle and laser treatment (FIG. 31a). RT-PCR analysis of biomarkers for all three germ layers confirms the delay at the mRNA level of untreated cells (FIG. 31b). Moreover, both ICC and RT-PCR analysis demonstrate that treatment using HRT and NIR laser does not change the ability of H9 cells to differentiate into all three germ layers (FIG. 31).

In summary, this disclosure describes the successful development of a strategy to control RNAi in human embryonic stem cells using a near-infrared laser. The modular nanocarrier efficiently penetrated into a broad range of hESC including H1, H7, H9, and H14. Internalization of the constructs was tracked and quantified by flow cytometry. Exposure to femtosecond pulses of NIR light caused knockdown of model targets GFP and Oct4 in hESC and, in the case of Oct4 knockdown, differentiation to all three germ layers, supporting the biocompatibility of this novel method. This strategy enables laser printing of siRNA in stem cells with cellular-level resolution at a desired time. The strategy therefore provides a tool useful for stem cell basic research and stem cell tissue engineering for regenerative medicine.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Nanoshell Synthesis

HGN were synthesized by galvanic replacement of silver seeds, as described previously (Wu et al., 2009, Chapter 14 Synthesis, Characterization, and Optical Response of Gold Nanoshells Used to Trigger Release from Liposomes. 2009, 464:279-307). Silver seed particles were prepared by reducing a stirred solution of 500 mL of 0.2 mM AgNO₃ (Sigma-Aldrich, St. Louis, Mo.) with 0.5 mL of 1.0 M NaBH₄ (EMD Millipore, Billerica, Mass.) in the presence of 0.5 mM sodium citrate (Sigma-Aldrich, St. Louis, Mo.) in deionized water. The solution was stirred for two hours at 60° C. before growing the seed particles to a final target size for use as a sacrificial template for the gold nanoshells growth by adding 0.75 mL of 2 M NH₂OH.HCl (Sigma-Aldrich, St. Louis, Mo.) and 1.75 mL of 0.1 M AgNO₃ and stirred overnight at room temperature. The galvanic replacement of the silver template particles with gold was optimized to have an absorbance peak at around 800 nm by rapid addition of 3.2 mL of 25 mM HAuCl₄ (Sigma-Aldrich, St. Louis, Mo.) at 60° C.

### HGN Characterization

To measure the size distribution of the hollow gold nanoshells, the particles were visualized by transmission electron microscopy using a FEI Tecnai G2 Sphera microscope operating at 200 kV. The optical properties were characterized by UV-Vis absorption using a Tecan Infinite 200 Pro microplate reader on a 96-well flat clear bottom plastic plate (BD Biosciences, San Jose, Calif.). Using a Nanosight LM10HS (Nanosight, Amesbury, UK) we estimated the hollow gold nanoshells to have an extinction coefficient of 3.1×10¹⁰ M⁻¹ cm⁻¹ at the plasmon peak wavelength of 800 nm.

### Preparation of NTA-HGN

Single-stranded 25-mer DNA (GCCACCACGTCTACTTGAAGTCCCA, SEQ ID NO:5) modified to possess NH₂-(CH₂)₇— at the 3′ end and -PEG18-(CH2)₆—SS—(CH₂)₆—OH at the 5′ end was purchased from Biosearch Technologies, Inc. (Petaluma, Calif.) and adsorbed onto hollow gold nanoshells as previously described (Wu et al., 2009, Chapter 14 Synthesis, Characterization, and Optical Response of Gold Nanoshells Used to Trigger Release from Liposomes. 2009, 464:279-307). 100 μM DNA was incubated with 10 mM Tris(2-carboxyethyl)phosphine hydrochloride (TCEP) (Sigma-Aldrich, St. Louis, Mo.) for 30 minutes and then dialyzed against TE buffer pH 8.0 overnight at 4° C. A final concentration of 3 μM TCEP-treated DNA was added to 32 pM hollow gold nanoshells at which point low pH adsorption was induced with the addition of 10 mM sodium citrate at pH 3.1. After 10 minutes, the pH was neutralized to 7.4 with the addition of 1 M HEPES (Sigma-Aldrich, St. Louis, Mo.) and a final NaCl concentration of 500 mM. The hollow gold nanoshells were then pelleted by centrifugation three times at 9000×g for 10 minutes, decanted and the pellet resuspended in 500 mM NaCl in 1 mM phosphate buffer at pH 7.4 to remove excess DNA. N—[Nα,Nα-Bis(carboxymethyl)-L-lysine]-12-mercaptododecanamide (NTA) (Sigma-Aldrich, St. Louis, Mo.) was then conjugated to the 3′ amine of the DNA scaffold by an NHS-PEG12-maleimide linker (Quanta Biodesign, Ltd., Plain City, Ohio). The solution was stored at 4° C. until use.

Construction, Expression and Purification of RPARPAR Modified GFP and Loading onto HGN.

Plasmid pRSET-EmGFP was purchased from Invitrogen (Life Technologies, Thermo Fisher Scientific, Inc., Waltham, Mass.). The vector contained a poly-histidine tag at the N-terminus of GFP. Oligonucleotides encoding the RPARPAR peptides were synthesized and ligated downstream of GFP, with a glycine-serine linker placed in between. RPARPAP-modified GFP fusion protein was confirmed by DNA sequencing. Protein was expressed in Escherichia coli BL21(DE3) (Novagen, EMD Biosciences, Inc., Billerica, Mass.) and purified using nickel-nitrilotriacetic acid affinity chromatography under native conditions. Purified protein was dialyzed in 10 mM Tris-HCl pH 7.4.

GFP was loaded unto the hollow gold nanoshells at a 100,000 to 1 GFP:HGN molar ratio in the presence of 400 μM NiCl₂ and incubated for 30 minutes on ice. The hollow gold nanoshells were pelleted by centrifugation at 9000×g for 10 minutes at 4° C. for a minimum of five times to remove excess GFP and resuspended in PBS containing 500 mM NaCl and 0.001% Tween 20 (Sigma-Aldrich, St. Louis, Mo.) at a final concentrate of 320 pM.

### Femtosecond Laser for GFP Release Quantification.

Samples were irradiated using a femtosecond Ti:sapphire regenerative amplifier running at 1 kHz repetition rate. The laser beam was collimated by a Galilean telescope to achieve a Gaussian diameter of 2.3 mm. In experiments without collimation, the full beam diameter was 5 mm. Pulse duration was monitored by a homebuilt single-shot optical autocorrelator and was kept at about 130 fs. The spectral full width at half maximum of the laser radiation was 12 nm centered around 800 nm. The laser beam was directed onto the sample by a series of mirrors, and no focusing optics were used. The energy of the optical pulse was controlled by Schott neutral density glass filters. A thermopile power meter (Newport Inc., Irvine, Calif.) was used to measure the incident optical power.

### Microplate Fluorescence Measurements of GFP.

Fluorescence measurements of GFP were carried out using a microplate reader (INFINITE 200 Pro, Tecan Group AG, Mannedorf, Switzerland), exciting at 450 nm (9 nm bandwidth) and reading emission spectra at 490 to 600 nm (20 nm bandwidth). The amount of GFP loaded unto NTA-HGN was determined by chemically competing for the nickel-NTA sites of 3.2 pM nanoparticles with 1 mM imidazole and incubation for 30 minutes. The particles were spun down at 12,000×g for 10 minutes and the supernatant was loaded into a 96-well flat clear bottom plastic microtiter plate for fluorescence readout. Efficiency of GFP release by laser was examined by irradiating a number of samples of 3.2 pM GFP-HGN with multiple combinations of laser powers and exposure times. Hollow gold nanoshells were then centrifuged at 12,000×g and the supernatants transferred to a 96-well flat clear bottom plastic plate for fluorescence readout.

### Cell Culture and GFP-HGN Incubation.

PPC-1 and M21 cells were maintained Dulbecco's Modified Eagle Medium (DMEM) high glucose medium with phenol red (HyClone, GE Healthcare Bio-Sciences, Pittsburgh, Pa.) supplemented with 10% fetal bovine serum (HyClone, GE Healthcare Bio-Sciences, Pittsburgh, Pa.) at 37° C. in 5% CO₂. Prior to co-culture, M21 cells were grown in 12 well plates and stained with 50 μM of CELLTRACKER Orange (CTO, Life Technologies, Thermo Fisher Scientific, Inc., Waltham, Mass.) for 30 minutes at 37° C. and 5% CO₂, according to the manufacturer's protocol, in serum-free medium. CTO becomes impermeable to the plasma membrane once internalized in the cytosol and is passed to daughter cells at division. M21 cells were then washed three times with Dulbecco's phosphate buffered saline (DPBS), collected using a 0.25% trypsin/EDTA solution (Sigma-Aldrich, St. Louis, Mo.), and plated as stated above. In co-culture experiments the cells were grown together on an 8-well chambered glass slide (LABTEK II, Thermo Fisher Scientific, Inc., Waltham, Mass.) at an initial seeding density of 20,000 PPC-1 and 20,000 M21 (pre-labeled with CTO) cells per well and then incubated for 24 hours at 37° C. in 5% CO₂ in complete medium. For spatial and temporal controlled release experiments PPC-1 cells were grown on an 8-well chambered glass slide (LABTEK II, Thermo Fisher Scientific, Inc., Waltham, Mass.) at an initial seeding density of 40,000 cells per well for 24 hours 37° C. in 5% CO₂ in complete media.

1 to 10 μL of GFP-HGN at 320 pM was added per 100 μL of medium per well. After two hours of incubation at 37° C. in 5% CO₂ atmosphere, the cells were rinsed with Hank's Balanced Salt Solution (HBSS) (Thermo Fisher Scientific, Inc., Waltham, Mass.) prior to imaging.

### Two-Photon and Confocal Microscopy.

Imaging was performed using a two-photon microscopy system (FLUOVIEW 1000, Olympus America, Inc., Center Valley, Pa.). Living cells were excited with a mode-locked titanium-sapphire tunable (690-1020 nm) femtosecond pulsed laser (MaiTai HP, Newport Corp., Irvine, Calif.) regulated with a modulator linked to the FLUOVIEW software (Olympus America, Inc., Center Valley, Pa.). A 25× water immersion objective with a numerical aperture of 1.05 was used. Images were collected on a 12 bit file with 512×512 pixels. GFP fluorescence in cells plated on an 8-well glass slide was imaged with a 15 mW blue laser diode exciting at 473 nm raster scanning at a speed of 80,000 Hz. near-infrared excitation of GFP-HGN was performed using the femtosecond pulsed Mai-Tai laser tuned to 800 nm at a raster scan speed of 125,000 Hz up to 35 full-frame cycles. Cells were then imaged again with the 473 nm laser diode. Spatially-controlled release experiments were performed by selecting a region of interest using the FLUOVIEW software to scan only the selected area with the femtosecond pulsed laser.

Visualization for co-culture utilized both 473 nm blue and 559 nm green laser diodes (15 mW) sequentially to excite GFP and CTO respectively. Cells containing GFP-HGN are pseudo-colored green whereas cells containing CTO are pseudo-colored red, unless otherwise stated.

### Image Analysis for Quantification of GFP Release by Microscopy.

Ratio quantification was performed using the Ratio Plus plugin in ImageJ (Schneider et al., 2012, Nature methods 9(7):671-675) to calculate the pixel ratio between images corresponding to after and before near-infrared excitation. Background subtraction of the pixels was assigned by determining the average background intensity. Line intensity quantification was performed on ImageJ.

### Example 2

### Gold Nanoshell Synthesis

Hollow gold nanoshells (HGN) were synthesized as described previously (Prevo et al., 2008, Small 4:1183-1195; Wu et al., 2008, J. Am. Chem. Soc. 130(26):8175-8177). Silver seed particles were prepared by reducing a stirred solution of 500 mL of 0.2 mM AgNO₃ with 0.5 mL of 1.0 M NaBH₄ in the presence of 0.5 mM sodium citrate in deionized water. The solution was stirred for two hours at 60° C. to allow NaBH₄ to fully hydrolyze. Larger silver nanoparticles to be used as sacrificial templates for HGN were grown from the silver seed solution by adding 0.75 mL of 2 M NH₂OH HCl and 1.75 mL of 0.1 M AgNO₃ and stirring overnight. The galvanic replacement of the silver template particles with gold was optimized to have an absorbance peak at around 800 nm by quickly mixing 3.2 mL of 25 mM HAuCl₄ at 60° C. The solution was cooled to room temperature (RT) and kept in dark for at least several days to allow the excess Ag⁺ that formed AgCl to settle to the bottom. HGN was then placed in a 3500 MWCO SLIDE-A-LYZER (Thermo Fisher Scientific Inc., Rockford, Ill.) in 3500 mL of 500 μM citrate buffer at pH 5.5. 0.1% of diethylpyrocarbonate (DEPC, Sigma-Aldrich, St. Louis, Mo.) was added to neutralize any RNase activity, and stirring for two days at room temperature allows the DEPC to decompose. TEM imaging was performed using a TECNAI G2 Sphera microscope (200 kV; FEI, Hillsboro, Oreg.). Size distribution analysis was done by dynamic light scattering (DLS) with a Nano ZS zetasizer (Malvern Instruments, Ltd., Malvern, UK) with autotitrator instrument. Optical characterization was performed by UV-VIS spectrophotometry with a UV-1700 instrument (PharmaSpec, Birmingham, UK). Particle concentration was estimated using the nanoparticle-tracking analysis system LM10HS (NanoSight Ltd., Amesbury, UK).

### Anchoring DNA or RNA Preparation

The siRNA sequence (sense: RNA-3, Table 1; anti-sense: RNA-2, Table 1) was used previously to knockdown plk1 gene expression. (Dassie et al., 2009, Nat. Biotechnol. 27:839-849). Modified sense RNA strand RNA-1, control DNA strand DNA-1, and anchoring DNA strand for modular design DNA-4 are shown in Table 1. The RNA strand was dissolved in water (RNase-free) to a concentration of 100 μM and aliquoted at 200 μL volume per tube (1.5 mL RNase-free Eppendorf) followed by vacuum evaporation. The dried RNA was treated with 100 μL of 100% ethanol and stored at −80° C. On thawing, RNA disulfide protection was removed by first vacuum-evaporating the ethanol and then adding 200 μL of water and 5 μL Tris(2-carboxyethyl)phosphine HCl at pH 7.0 (TCEP, 0.5 M, #646547-10×1 mL; Sigma-Aldrich, St. Louis, Mo.). After 10 minutes, CHCl₃ (800 μL) was mixed to extract and remove the mercaptohexanol, carefully discarding the organic phase, and repeating for a total of four extractions. The aqueous layer was transferred to a new tube and used immediately.

TABLE 1
Oligonucleotides* used

| Oligos | 5′ | Sequence (5′→3′) | 3′ | Use |
|---|---|---|---|---|
| RNA-1 | Thiol- | GGGCGGCUUUGCCAAGUGCUU | NH₂ | Short RNA anchor (sense) |
| | PEG18 | | | |
| RNA-2 | — | AAGCACUUGGCAAAGCCGCCCUU | — | Knockdown control (anti-sense) |
| RNA-3 | — | GGGCGGCUUUGCCAAGUGCUU | — | Knockdown control (sense) |
| RNA-4 | Quasar | AAGCACUUGGCAAAGCCGCCCUU | — | Knockdown control/ |
| | 570 | | | Quantification/Imaging |
| RNA-5 | — | UGGGACUUCAAGUAGACGUGGUGGCUU | — | Modular design test |
| | | AAGCACUUGGCAAAGCCGCCCUU | | |
| RNA-6 | — | UGGGACUUCAAGUAGACGUGGUGGCUU | — | Modular design test |
| | | AAGGGCGGCUUUGCCAAGUGUUU | | |
| RNA-7 | Quasar | GGGCGGCUUUGCCAAGUGCUU | — | Modular design |
| | 570 | | | quantification/knockdown |
| RNA-8 | Quasar | GCACUUGGCAAAGCCGCCCUU | — | Modular design |
| | 570 | | | quantification/knockdown |
| DNA-1 | Thiol- | ACCCTGAAGTTCATCTGCACCACCG | NH₂ | Short DNA anchor |
| | PEG18 | | | |
| DNA-2 | FAM | CGGTGGTGCAGATGAACTTCAGGGT | — | Knockdown control/ |
| | | | | Quantification |
| DNA-3 | Thiol- | ACCCTGAAGTTCATCTGCACCACCG | Quasar | Anchoring quantification |
| | PEG18 | | 570 | |
| DNA-4 | NH₂ | GCCACCACGTCTACTTGAACTCCCA | Thiol- | Modular design anchor |
| | | | PEG18 | |

*all nucleotides obtained from Biosearch Technologies, Inc., Petaluma, CA

TABLE 2

| | |
|---|---|
| 5′ Thiol- PEG18 | 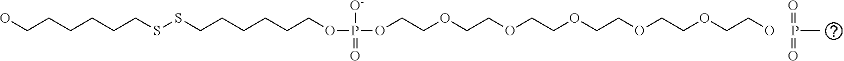 |
| 3′ NH₂ | 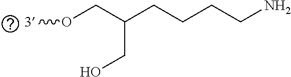 |
| 5′ NH₂ | 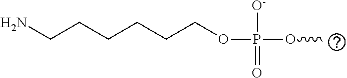 |
| 3′ Thiol- PEG18 | 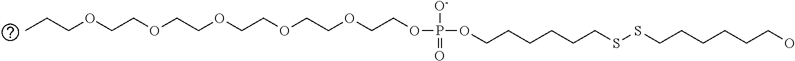 [SMILES: CCCOCCOCCOCCOCCOCCOP(=O)([O-])OCCCCCCSSCCCCCCO] |
| 5′ Quasar 570 | 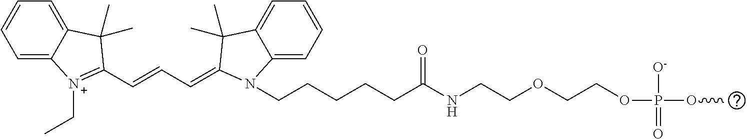 |
| 3′ Quasar 570 | 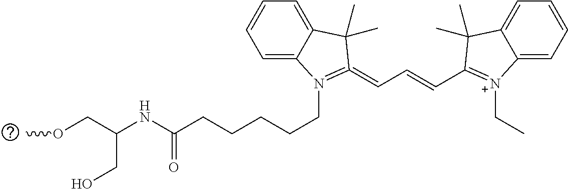 |
| 5′ FAM | 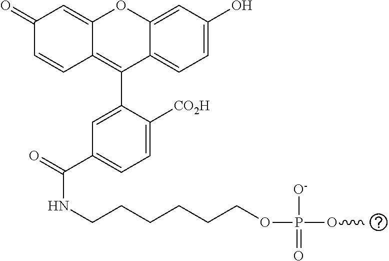 |

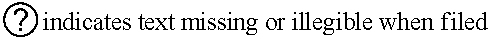

### HGN-Anchoring DNA or RNA Assembly

Thiol-modified sense RNA or DNA strands were assembled onto the HGN using a fast pH-induced self-assembly method. (Zhang et al., 2012, J. Am. Chem. Soc. 134:7266-7269). Approximately 0.1 nM HGN were combined with 9 μM freshly reduced thiol-modified DNA or RNA (100 μM in 12.5 mM TCEP pH7.0) and 10 mM sodium citrate-HCl (Na₃Cit-HCl, 500 mM, pH 3.0, RNase free), sonicated, and incubated at room temperature for 20 minutes. Thereafter, the solution was pH-neutralized by adding 130 mM HEPES buffer (1 M, RNase free, pH 7.5, Ambion, Life Technologies, Thermo Fisher Scientific, Inc., Waltham, Mass.), then salted to 1 M Na⁺ in steps, using 3.0 M NaCl, 0.3 M Na₃Cit pH 7.0 (20×SSC, RNase free, Promega Corp., Madison, Wis.), in approximately 300 mM Na⁺ increase per step, waiting five minutes between each step. 1 mM MgCl₂ (1 M, RNase free, TEKNOVA) and 0.01% Tween-20 (RNase free, Promega Corp., Madison, Wis.) were added and incubated overnight at RT to increase DNA or RNA density on the HGN. The next day HGN-Anchoring DNA or RNA were washed by centrifuging twice at approximately 7000×g for 10 minutes, each time keeping the pellet and redispersing with washing buffer (1 mM MgCl₂, 0.01% Tween-20, 300 mM NaCl, 30 mM Na₃Cit pH 7.0).

HGN-dsDNA or siRNA Duplex Hybridization and Targeting Peptide Conjugation

Anti-sense strand RNA-2 or RNA-4 (Table 1), complementary DNA strands for control DNA-2 (Table 1), or complementary RNA strands for modular design RNA-3 and RNA-5 (Table 1) were added at 3 μM to a washed stock of 1 mL HGN-anchoring DNA or RNA (0.1 nM) and incubated at 70° C. for two minutes followed by 45° C. for 30 minutes. Excess DNA or RNA was removed with conjugation washing buffer (100 mM HEPES pH 7.5, 1 mM MgCl₂, 0.01% Tween-20). Thiol-PEG-amine (3 kDa, Rapp Polymere GmbH) in ethanol was added at 100 μM to backfill any large exposed surface sites. After one hour, excess thiol-PEG-amine was removed by centrifugation at approximately 7000×g for 10 minutes and the pellet was resuspended in conjugation wash buffer. 6-mercapto-1-hexanol (MCH, Sigma-Aldrich, St. Louis, Mo.) in ethanol was added at 5 μM to further passivate HGN surface sites. After three hours, HGN-dsDNA or HGN-siRNA were centrifuged at approximately 7000×g for 10 minutes and then washed with conjugation wash buffer to remove excess MCH. Thereafter, a large excess (˜1 mg/mL) of MAL-dPEG₄-NHS linker (Quanta BioDesign, Ltd., Powell, Ohio; CAS#756525-99-2) for conjugation of the HGN-dsDNA or siRNA and the targeting peptide was added to functionalize the 3′ end of anchoring the DNA or RNA. The solution was sonicated briefly and incubated for 15 minutes at room temperature, followed by centrifugation at approximately 7000×g for 10 minutes at 4° C. and the pellet was resuspended with conjugation wash buffer twice to remove excess linker. 20 μM of FAM-Cys-X-RPARPAR-OH peptide (RP) (LifeTein LLC, Hillsborough, N.J.) was then added, the solution was briefly sonicated and incubated at room temperature for one hour. In the FAM-Cys-X-RPARPAR-OH peptide, X is an aminohexanoic linker, FAM is fluorescein attached to the N-terminus, and the C-terminus is free carboxyl. The dsDNA-coated or siRNA-RP-coated HGN were centrifuged twice at approximately 7000×g, the pellet was redispersed in conjugation wash buffer to remove any unreacted peptide, and sterile-filtered through a 0.22 μm syringe filter (Millpore Corp., Billerica, Mass.). The solution was then concentrated approximately five-fold (10 nM HGN) by centrifugation at approximately 7000×g for 10 minutes with resuspension of the pellet in conjugation wash buffer. Product was stored at 4° C. before adding to cells.

### Cell Culture

The human prostate cancer cells (PPC-1) were a generous gift from Erkki Ruoslahti (Sanford-Burnham Medical Research Institute, La Jolla, San Diego, Calif.). They were grown in DMEM/high glucose medium with phenol red (Hyclone, GE Healthcare Bio-Sciences, Pittsburgh, Pa.), supplemented with 10% FBS (HyClone, GE Healthcare Bio-Sciences, Pittsburgh, Pa.). Noncancerous prostate epithelial cells (RWPE-1, ATCC Accession No. CRL-11609) were grown in keratinocyte serum free medium (Invitrogen, Life Technologies, Thermo Fisher Scientific, Inc., Waltham, Mass.) supplemented with bovine pituitary extract (0.05 ng/mL) and recombinant EGF (5 ng/mL). Both cell lines were maintained at 37° C. in 5% CO₂ atmosphere and grown in 6-well or 96-well plates (Falcon, BD Biosciences, San Jose, Calif.) for experiments.

siRNA Transfection with LIPOFECTAMINE RNAiMAX

For the typical control transfection experiment with non-HGN conjugated siRNA duplex, cells were plated in 96-well plates at a concentration of 3000 cells per well. The following day, the reagents were prepared for transfection according to the manufacturer's protocol, with the following quantities intended per well in a 96-well plate format: 0.1 μL of LIPOFECTAMINE RNAiMAX and 0.1 μL of 5 μM siRNA were each diluted in 10 μL of OPTI-MEM reduced serum medium (Invitrogen, Life Technologies, Thermo Fisher Scientific, Inc., Waltham, Mass.), then combined and incubated at room temperature for 15 minutes. The transfection mix was then added to the wells containing the plated cells and complete growth medium. The following day the medium was replaced and cells imaged, scored for viability or allowed to grow up to 48 hours or 72 hours after transfection.

### HGN Transfection and Femtosecond Laser Irradiation

Cells were plated in 6-well plates (24 hours before experiment) and harvested by incubation with 500 μL of non-enzymatic cell dissociation buffer (CDB, Invitrogen, Life Technologies, Thermo Fisher Scientific, Inc., Waltham, Mass.) at 37° C. in 5% CO₂ atmosphere for 10-15 minutes, after one wash in calcium and magnesium free Dulbecco's phosphate buffered saline (D-PBS, Invitrogen, Life Technologies, Thermo Fisher Scientific, Inc., Waltham, Mass.). Complete growth medium was added and cells centrifuged at 1000×g to remove the CDB. The cell pellet was diluted in the appropriate amount of medium to obtain a concentration of 1×10⁶ cells/mL. 6.5 pM of coated HGN were added to 200 μL of cell suspension and incubated in 1.5 mL Eppendorf tubes at room temperature (RT) for two hours on a rotator. 1.2 mL of cold Hank's balanced saline solution (HBSS, with Ca²⁺ and Mg²⁺, pH 6.7-7.8, Invitrogen, Life Technologies, Thermo Fisher Scientific, Inc., Waltham, Mass.) was added and the tube was centrifuged at 55×g for eight minutes at 4° C. The supernatant containing free particles was removed and 45 μL HBSS were added and mixed with the cell pellet. Tubes were irradiated with 2.4 W/cm² pulsed NIR laser for 10 seconds by the output of femtosecond (fs) Ti: sapphire regenerative amplifier (Spitfire, Spectra-Physics, Santa Clara, Calif.) running with 1 kHz repetition rate. The laser beam with ˜4 mm diameter was directed onto the sample by a system of mirrors without any focusing optics. The pulsed duration was monitored by a home-built single-shot optical autocorrelator and was kept at about 130 fs. The spectral range of laser irradiation was approximately 12 nm centered around 800 nm, and the energy of the optical pulse was controlled by Schott neutral density glass filters. A thermopile power meter (Newport Inc., Irvine, Calif.) was used to measure the incident optical power. Cells were then either collected for fluorescence intensity measurement by flow cytometry, or plated in 96-well plates for cell viability assay and in 6-well plates for Western blot.

### Cell Fluorescence Intensity Measurements

Cells with internalized particles in HBSS buffer with or without femtosecond laser treatment were collected and injected into a flow cytometer (ACCURI C6, BD Biosciences, San Jose, Calif.) with a flow rate of 14 μL/min. The gate was based on the lineage area of forward and side scatter plots, and 10,000 events were collected for each sample. The increase in fluorescence intensity after particle internalization or laser treatment was assessed by the population of the cells having intensity higher than 99% of the control cells.

### Confocal Microscopy

Cells were plated on a 8-well LAB-TEK II chamber glass slide (Thermo Fisher Scientific, Waltham, Mass.), and incubated with 6.5 pM coated HGN at 37° C. in 5% CO₂ atmosphere for two hours followed by two washes with HBSS to remove any excess HGN. A FLUOVIEW 1000 MPE Microscope (Olympus America Inc., Center Valley, Pa.) with a 25× water immersion objective (NA 1.05) was used for live cell imaging. The microscope is equipped with a mode-locked titanium-sapphire femtosecond tunable pulsed laser (MAI TAI HP, Spectra-Physics, Santa Clara, Calif.), that was used to irradiate the sample to induce cargo release from the HGN, a 473 nm blue laser diode, used to image the FAM signal from the peptide, and a 559 nm green laser diode and a 633 nm HeNe laser that were not used. Images were collected at 12 bit with 512×512 pixels. The MAI TAI laser was tuned to 800 nm and used to irradiate the sample area with a scan speed of 125 k Hz for up to 35 repetitions. The specimen was imaged in a single-photon confocal mode with the blue laser diode at a scan speed of 80 k Hz before and after the exposure to the MAI TAI fs laser to compare the cell fluorescence intensity difference caused by the laser treatment.

### Cell Viability Assay

The PRESTOBLUE assay (Invitrogen, Life Technologies, Thermo Fisher Scientific, Inc., Grand Island, N.Y.) was used to determine the effect of transfection on cell proliferation for both laser treatment and controls with LIPOFECTAMINE RNAiMAX (Life Technologies, Thermo Fisher Scientific, Grand Island, N.Y.). For laser experiments, cells, with or without particles and treated or untreated with the laser, were plated in 96-well plates at a density of 5000 cells per well. 100 μL of complete medium were added per well and cells were incubated for 0 hours, 24 hours, 48 hours, or 72 hours, before determining cell viability. For LIPOFECTAMINE control experiments cells were tested for viability 24 hours, 48 hours, or 72 hours after transfection. The PRESTOBLUE assay was used according to the manufacturer's instructions: 10 μL of reagent were added to cells with 90 μL of fresh complete medium; the plate was incubated at 37° C., 5% CO₂ atmosphere for two hours; the fluorescence signal was recorded in a Tecan Infinite 200 Pro (Tecan Group AG, Mannedorf, Switzerland) reader in bottom-read mode. Excitation and emission wavelengths were set at 560 nm (9 nm bandwidth) and 600 nm (20 nm bandwidth) respectively. Four replicates for each treatment were averaged and analyzed based on a calibration curve to determine then number of cells in each sample. All treatments were repeated at least three times and reported as mean±standard deviation (SD). One-way ANOVA analysis was performed to determine the statistical significance of changes in cell viability for each treatment; p<0.001 was considered statistically significant.

### Western Blot

Cells, with or without particles and treated or untreated with laser, were plated in 6-well plates with 2×10⁵ cells per well in 2 mL medium and cultured for 48 hours or 72 hours. Cells were harvested with trypsin/EDTA (0.25%, Sigma-Aldrich, St. Louis, Mo.) and about 1×10⁶ cells were lysed in 100 μL of RIPA lysis buffer (50 mM Tris, 150 mM NaCl, 1% Triton, 0.5% deoxycholate, and 2 mM EDTA) supplemented with protease inhibitor cocktail (Promega Corp., Madison, Wis.) on ice for 30 minutes. The cell lysate was centrifuged at approximately 12400×g, at 4° C. for 20 minutes. The supernatant was collected and combined with loading buffer (6×, 300 mM Tris-HCl, 0.01% w/v bromophenol blue, 15% v/v glycerol, 6% w/v SDS and 1% v/v beta-mercaptoethanol) and kept at 95° C. for 10 minutes. 40 μL of this solution and 10 μL of pre-stained molecular weight standard (New England BioLabs Inc., Ipswich, Mass.) were loaded and separated by electrophoresis through precast 10% SDS-PAGE gel (Bio-Rad Laboratories, Inc., Hercules, Calif.). Proteins were electro-transferred onto a nitrocellulose membrane (Bio-Rad Laboratories, Inc., Hercules, Calif.). The membrane was blocked with 5% bovine serum albumin (BSA, Sigma-Aldrich, St. Louis, Mo.) in PBST (PBS, 0.1% Tween-20) at room temperature for 30 minutes, then incubated overnight at 4° C. with primary antibodies diluted in 5% BSA-PBST buffer: mouse anti-PLK1 (monoclonal, 1:500 dilution, EMD) and rabbit anti-β-actin (monoclonal, 1:1000 dilution, Abcam plc, Cambridge, Mass.). The membrane was subsequently washed thrice for 15 minutes using PBST and then incubated for three hours in 5% BSA-PBST with secondary antibodies including Alexa Fluor 488 labeled goat anti-mouse IgG (1:10,000 dilution, Invitrogen, Life Technologies, Thermo Fisher Scientific, Inc., Waltham, Mass.) and Alexa Fluor 647 labeled goat anti-rabbit IgG (1:10,000 dilution, Invitrogen, Life Technologies, Thermo Fisher Scientific, Inc., Waltham, Mass.), followed by wash three times for 15 minutes each using PBST. Images were acquired using a GE Healthcare Typhoon 9400 scanner system and the bands were analyzed using the scanner control software.

### Particle Concentration Determination

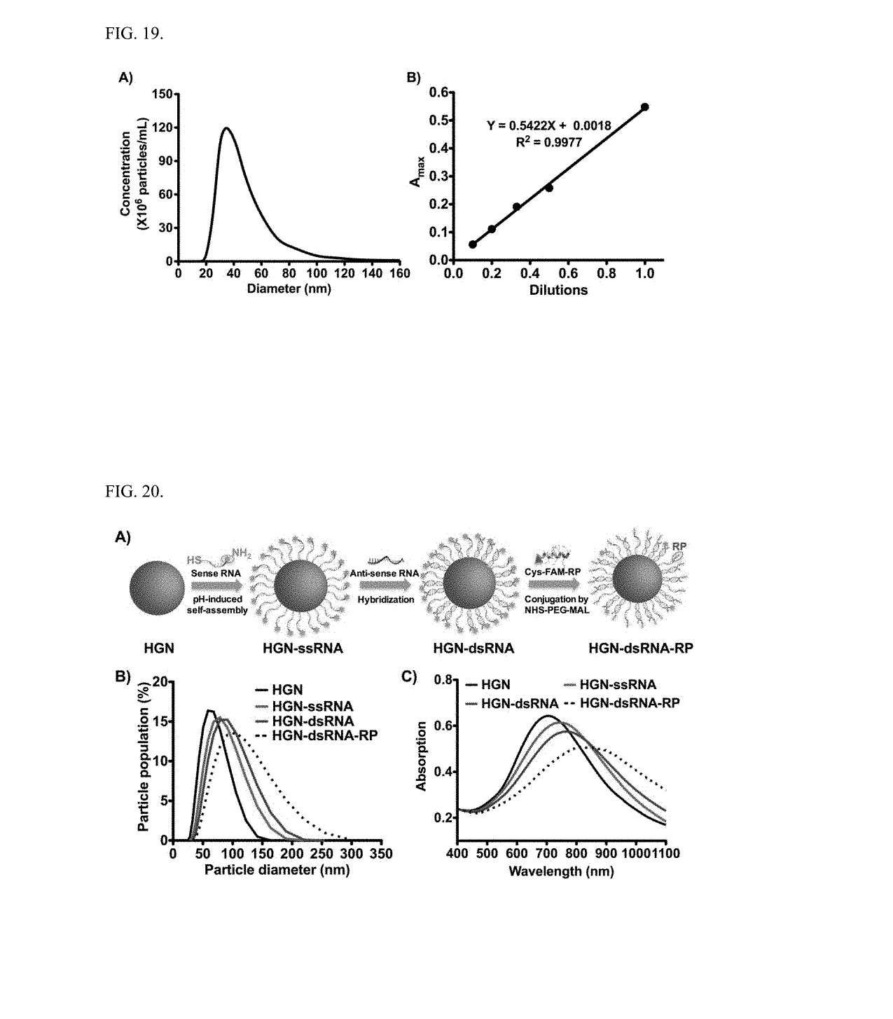

Particle concentration was estimated using an LM10HS nanoparticle-tracking analysis system (NanoSight Ltd., Amesbury, UK), equipped with an LM14 viewing unit (NanoSight Ltd., Amesbury, UK) with a 638 nm laser and a VITON fluoroelastomer O-ring (E. I. du Pont de Nemours and Co., Wilmington, Del.). The samples are injected in the sample chamber at room temperature with sterile KENDALL syringes (Covidien, Mansfield, Mass.) until the liquid reached the tip of the nozzle. The software used for capturing and analyzing the data is the NTA 2.3 Build 0025. The samples are measured for 60 s with manual camera level adjustments. The NanoSight system shows excellent accuracy by calibrating with monodisperse 50 nm and 60 nm diameter solid gold nanoparticles with known concentrations (Ted Pella Inc, Redding, Calif.). HGN with maximum absorption at 2.0 optical density (1.0 cm path length) was determined by NanoSight to contain 3.9×10⁹ particles per milliliter. A linear correlation is observed when measuring the maximum absorption of HGN with a serial of dilutions, suggesting that HGN follows the Beer-Lambert Law. Particle concentration after coating was estimated to have an extinction coefficient of 3.1×10¹⁰ M⁻¹ cm⁻¹ at the NIR plasmon peak maximum absorbance (FIG. 19).

### Nanocarrier (HGN-SD-RP) Optical Characterization at Different Coating Steps

Sense RNA strand modified with thiol-PEG on the 5′ and with amine on the 3′ end was assembled on HGN through a fast pH-induced self-assembly method. Thereafter anti-sense RNA strand was hybridized onto HGN-ssRNA to form functional plk1-siRNA duplex. Targeting peptide was Cys-FAM-RP (FAM-Cys-X-RP-OH, X being aminohexanoic linker, and FAM is fluorescein coupled to the N-terminus through an amide bond, and RP is SEQ ID NO:1). Cys-FAM-RP with free thiol was tethered to the 3′ end of siRNA sense strand with the help of a NHS-(PEG)₄-MAL linker (CAS#756525-99-2, FIG. 20A). The diameter of the construct was monitored during assembly by DLS. Citrate-stabilized HGN, as synthesized, had an average diameter of 56 nm (FIG. 20B). The addition of the anchoring strand and subsequent hybridization of RNA caused a radial increase of ˜8 nm (FIG. 20B). The value was in agreement with the theoretical length of dsRNA (8 nm), and was consistent with a compactly arranged layer perpendicular to the gold surface. The PEG and peptide coating step further increased the particle diameter from 73 to 89 nm (FIG. 20B). A small amount of aggregation was observed from the comparatively wider particle size distribution peak (FIG. 20B).

Quantification of siRNA Coating Density on HGN and NIR Laser-Dependent Release

The coating density of the siRNA short duplex (SD) on HGN was estimated by native-PAGE. The siRNA-coated nanoparticles were etched by KCN to completely release the oligonucleotides. The chemically released siRNA from HGN was loaded into a native-PAGE gel. The gel was then stained in SYBR Gold (Invitrogen, Life Technologies, Thermo Fisher Scientific, Inc., Waltham, Mass.), followed by densitometry analysis of the gel-scanning image. A calibration curve was generated by loading a known concentration gradient of siRNA in the gel and performing densitometry analysis of the bands (FIG. 21). Using the reported calibration curve and the measured HGN concentration, the number of siRNA short duplex strands per HGN was estimated as 2300±600.

The coating density of the long duplex (LD) siRNA on HGN was estimated using a fluorescence-based method, relatively to the SD siRNA. 5′ Quasar570 (Q)-labeled antisense strand (Table 1, RNA-4) and 5′ Q-labeled sense strand (Table 1, RNA-7) were hybridized to form SD and LD respectively. The siRNA (SD and LD)-coated nanoparticles were etched by KCN to completely release the oligonucleotides. The fluorescence intensity of the SD solution after KCN etching was measured and the fluorescence per HGN was calculated and considered to be 1. The fluorescence intensity of the LD solution after KCN etching was measured and the contribution per HGN found to be 60% of the SD intensity. Similarly, the efficiency of NIR laser release of siRNA from HGN surface was assayed by measuring the fluorescence in solution after laser release, in comparison with that from KCN etching.

### Minimum NIR Laser Power and HGN-SD-RP Dosage for Effective Knockdown

Different NIR laser power intensity and irradiation duration combinations were applied on the PPC-1 cells internalized by HGN-SD-RP or HGN-dsDNA-RP. Cell viability after 72 hours was tested to separate the released-siRNA biological effect from nanoparticle local heating damage. Cell death from HGN-dsDNA-RP would indicate particle local heating damage to the cells, since the construct is not biologically active. On the other hand, cell death from HGN-siRNA-RP is the result of siRNA knockdown. 2.4 W/cm² for 10 seconds was the optimal laser condition for effective siRNA knockdown with minimum local heating damage.

### Example 3

### Cell Culture

The human embryonic stem cells H1, H7, H9 and H14 (WiCell Research Institute, Inc., Madison, Wis.) were maintained on MATRIGEL (BD Biosciences, San Jose, Calif.) coated 6-well plates (Falcon, BD Biosciences, San Jose, Calif.) with mTeSR1 medium (Stemcell Technologies, Inc., Vancouver, BC, Canada) at 37° C. in 5% CO₂. Cells were passaged by manual dissection without enzymatic dissociation every 5-7 days. For differentiation, H9 cells were cultured with differentiation medium (Dulbecco's modified Eagle's medium (DMEM)/F12, 20% knockout serum replacement, 0.1 mM MEM nonessential amino acid solution, and 0.1 mM β-mercaptoethanol (all from Invitrogen, Life Technologies, Thermo Fisher Scientific, Inc., Waltham, Mass.). HEK293T cells (CRL-11268) were maintained in DMEM supplemented with 10% fetal bovine serum. 50 μg/mL NORMOCIN (InvivoGen USA, San Diego, Calif.) was supplemented in cell culture media.

### Lentiviral Transfection to Generate Transduced 119-GFP and Knockdown Oct4 Gene Expression

Lentiviral vector pLVTHM-scramble (a gift from Dr. Zach Ma, University of California-Santa Barbara) containing a eGFP tag was used to generate a transduced H9-GFP cell line. Lentiviral vectors LL-hOCT4i-1 and LL-hOCT4i-2 (Cat. nos. #12197, #12198, Addgene, Cambridge, Mass.) were used for the expression of Oct4-shRNA in H9 cells. Lentiviral vector pLVTHM-scramble containing a non-targeting scrambled shRNA was also used as the negative control for Oct4 gene knockdown (shRNA: 5′-GCUUGUUCGUUGGUAACUACAUU-3′ (SEQ ID NO:18). Lentiviral vector plasmids along with psPAX2 (Cat. no. #12260, Addgene, Cambridge, Mass.) and pMD2.G (Cat. no. #12259, Addgene, Cambridge, Mass.) were transfected into HEK293T cells to generate the viral particles. After 48 hours and 72 hours, supernatants were collected, filtered through a 0.22 μm filter, and concentrated by PEG-it (System Biosciences, Inc., Mountain View, Calif.) precipitation and centrifugation at 1500×g for 30 minutes. A viral titer was performed by transducing Hela cells with several different dilutions of isolated virus. Passage 54-56 H9 cells were dissociated to cell aggregates (˜20 cells per aggregate) using cell dissociation buffer (CDB, Invitrogen, Life Technologies, Thermo Fisher Scientific, Inc., Waltham, Mass.) and plated on MATRIGEL-coated 6-well plates, and after seeding for one day, cells were incubated with concentrated virus for another 24 hours in the presence of 0.6 μg/mL polybrene (Millipore, Billerica, Mass.). Seven days post transduction, cell colonies were assayed for GFP or OCT4 protein expression. For generating stable H9-GFP cells, colonies with high GFP expression were manually dissected and screened for 2-3 passages.

siRNA Transfection with Commercial Transfection Reagents

Testing of commercially available transfection reagents LIPOFECTAMINE 2000 (Invitrogen, Life Technologies, Thermo Fisher Scientific, Inc., Waltham, Mass.), LIPOFECTAMINE RNAiMAX (Invitrogen, Life Technologies, Thermo Fisher Scientific, Inc., Waltham, Mass.) and JETPRIME (Polyplus-transfection, Inc., New York, N.Y.) in transduced H9-GFP cells was conducted by measuring GFP expression after GFP-siRNA (EGFP-S1 DS Positive Control, Integrated DNA Technologies, Inc., Coralville, Iowa) transfection. Cells were dissociated using CDB buffer treatment and plated on a 12-well plate (1×10⁵ cells per well) in the presence of ROCK Inhibitor (Y-27632, Millipore, Billerica, Mass.) at 10 μM. The following day the transfection was performed according to the manufacturer's protocol: 1 μL of LIPOFECTAMINE 2000 or LIPOFECTAMINE RNAiMAX or JETPRIME with 15 nM siRNA was added to each well following medium exchange (mTeSR1+10% FBS, no NORMOCIN (InvivoGen USA, San Diego, Calif.)). After 24 hours, the medium was replaced with fresh mTeSR1 medium. Cells were imaged by fluorescence microscopy and collected for flow cytometry three days after the transfection to assay GFP expression.

Hollow Gold Nanoshell Synthesis, Characterization and dsDNA/siRNA Assembly

Hollow gold nanoshells (HGN) were synthesized as previously described (Huang et al., 2014, Nano Lett. 14(4):2046-2051; Braun et al., 2009, ACS Nano. 3(7):2007-2015). Briefly, silver seeds were initiated by stirring 500 mL 0.2 mM AgNO₃ in the presence of 1.0 mM NaBH₄ and 0.5 mM sodium citrate at 60° C. for two hours; thereafter, the sacrificial larger particles were generated by adding 2.0 M NH₂OH:HCl and 0.1 M AgNO₃ at room temperature and stirred overnight. HGNs were then synthesized by galvanic replacement of silver template particles through the stepwise addition of 0.025 M HAuCl₄ to the nanoparticle solution, pre-heated to 60° C., followed by stirring for 3-4 hours until the plasmon peak reaches approximately 800 nm. After storing at room temperature for several days the solution was decanted to remove the silver chloride precipitates. The HGN solution was dialyzed against 500 μM citrate buffer at pH 5.5 supplemented with 0.1% of diethylpyrocarbonate (DEPC, Sigma-Aldrich, St. Louis, Mo.) in a 3500 MWCO Slide-A-Lyzer (Thermo Fisher Scientific, Inc., Waltham, Mass.) to remove the residual reactants and to neutralize any RNase activity. Optical characterization was assayed by UV-VIS spectrophotometry with a UV-1700, PharmaSpec instrument (Shimadzu Corp., Kyoto, Japan). Particle size was analyzed by dynamic light scattering (DLS) with a Nano ZS zetasizer (Malvern Instruments, Ltd., Malvern, UK). TEM images were collected using a TECNAI G2 Sphera microscope (200 kV) (FEI, Hillsboro, Oreg.).

The siRNA sequences (siGFP and siOCT4, Table 3) for GFP and Oct4 knockdown were adopted from previous reports (Braun et al., 2009, ACS Nano. 3(7):2007-2015; Zaehres et al., 2005, Stem Cells 23(3):299-2305). Modified DNA and RNA strands (Table 3) were purchased from Biosearch Technologies, Inc. (Petaluma, Calif.).

TABLE 3
Modified oligonucleotides used for HGN-dsDNA/siRNA assembly*

| Modified Oligos | 5′ | Sequence (5′→3′) | 3′ |
|---|---|---|---|
| siGFP sense | Thiol-PEG18 | ACCCUGAAGUUCAUCUGCACCACCG | NH₂ |
| siGFP anti- | Quasar570 | CGGUGGUGCAGAUGAACUUCAGGGUCA | — |
| sense | | | |
| siOCT4 sense | Thiol-PEG18 | GGAUGUGGUCCGAGUGUGUGGUUCG | NH₂ |
| siOCT4 anti- | Quasar570 | UUAACCACACUCGGACCACAUCCUU | — |
| sense | | | |
| DNA-1 | Thiol-PEG18 | ACCCTGAAGTTCATCTGCACCACCG | NH₂ |
| DNA-2 | Quasar570 | CGGTGGTGCAGATGAACTTCAGGGT | — |

*5′ Thiol-PEG18, 5′ Quasar570, and 3′ NH₂ modifications are as shown in Table 2.

The thiol-modified RNA strands (siGFP sense and siOCT4 sense) were dissolved in water (RNase-free) and aliquoted at 20 nmol per tube (1.5 mL RNase-free Eppendorf) followed by vacuum evaporation. The dried RNA aliquots were stored at −80° C. in the presence of 100% ethanol. Prior to use, thiol-RNA was re-dissolved in 200 μL water (RNase-free) after the removal of ethanol by vacuum-evaporation. The removal of the disulfide protecting group for thiol-DNA or thiol-RNA was initiated by adding 5 μL Tris(2-carboxyethyl)phosphine HCl at pH 7.0 (TCEP, 0.5 M, Sigma-Aldrich, St. Louis, Mo.) for 10 minutes followed by chloroform extraction (4×800 μL). Freshly reduced DNA or RNA strands with thiol modification were conjugated to the HGNs using a fast pH-induced self-assembly method as previously described (Huang et al., 2014, Nano Lett. 14(4):2046-2051; Zhang et al., 2012, J Am Chem Soc. 134(17):7266-7269). Approximately 0.1 nM HGN were combined with 9 μM thiol-DNA or thiol-RNA and 10 mM sodium citrate-HCl (Na₃Cit-HCl, 500 mM, pH 3.0, RNase free) and incubated at room temperature for 20 minutes after brief sonication. The pH was neutralized by the addition of 130 mM HEPES buffer (1M, RNase free, pH 7.5, Ambion, Life Technologies, Thermo Fisher Scientific, Inc., Waltham, Mass.), and a 1 M Na⁺ was added in 300 mM steps using 3.0 M NaCl, 0.3 M Na₃Cit pH 7.0 (20×SSC, RNase free, Promega Corp., Madison, Wis.) with five minutes incubation for each step. 0.01% Tween-20 (RNase free, Promega) and 1 mM MgCl₂ (1M, RNase free, Teknova, Hollister, Calif.) were added and incubated for one hour at room temperature to increase DNA or RNA density on the HGN. Unbound thiol-DNA or RNA strands in suspension were removed by washing the particles twice with assembly washing buffer (1 mM MgCl₂, 0.01% Tween-20, 300 mM NaCl, 30 mM Na₃Cit pH 7.0). All the nanoparticle washing steps were performed by centrifuging at 7000×g for 10 minutes and resuspending the pellet in the respective buffer outlined below through brief sonication.

3 μM anti-sense RNA strands or complementary DNA strand DNA-2 (Table 3) were added to 1 mL HGN-thiolated DNA or RNA (0.1 nM) in the presence of 10 mM Mg²⁺ and 600 mM Na^{t}, and incubated at 70° C. for two minutes followed by 45° C. for 30 minutes. Excess DNA or RNA was removed with conjugation wash buffer (10 mM HEPES pH 7.5, 1 mM MgCl₂, 0.01% Tween-20). The remaining exposed surface of HGN was backfilled by adding thiol-PEG-amine (3 kDa, Rapp Polymere GmbH, Tubingen, Germany) at 100 μM and incubating for one hour, and was further passivated by adding 6-mercapto-1-hexanol (MCH, in ethanol, Sigma-Aldrich, St. Louis, Mo.) at 5 μM and incubating for three hours. Between and after these two steps, the particles were washed in conjugation wash buffer to remove the excess thiol-PEG-amine or MCH.

### TAT-Peptide Coating on HGN-dsDNA/siRNA

A large excess (˜1.5 mg/mL) of NHS-PEG4-Biotin (Thermo Fisher Scientific, Inc., Waltham, Mass.) dissolved in 50 μL DMSO was added to 1 mL ˜0.1 nM HGN-dsDNA or siRNA to functionalize the 3′ end of thiol-DNA or -RNA with biotin. The solution was sonicated briefly and incubated for one hour at room temperature, followed by washing with conjugation wash buffer twice to remove excess functionalizing reagent. All the nanoparticle washing steps were performed by centrifuging at 7000×g for 10 minutes and resuspending the pellet in the respective buffer outlined below through brief sonication. Streptavidin (PROzyme, Inc., Hayward, Calif.) was then coated on biotinylated oligonucleotides on HGN to bridge between nucleic acid and biotinylated TAT-peptide, by adding at 1 mg/mL to ˜0.05 nM nanoparticle in the presense of 0.5×PBST (DPBS with 0.1% Tween-20) and incubating at room temperature for one hour after brief sonication. To avoid the particle self-aggregation that may be caused by streptavidin bridging, the solution was vortexed and sonicated immediately upon the addition of streptavidin. The sequential washing of particles with two kinds of buffer (assembly washing buffer and conjugation washing buffer) enhanced the nanoparticle monodispersity. HGN-dsDNA or siRNA with streptavidin coating was finally coated with biotin-TAT (N-terminal biotin, YGRKKRRQRRR (SEQ ID NO:4), C-terminus free GenScript) to form the multivalent TAT-peptide layer on the outside, of which biotin-TAT was added to ˜0.05 nM nanoparticle twice at 15 μM in the presence of 0.5×PBST followed by brief sonication and 30 minutes incubation at RT. The nanoparticles were then sequentially washed with assembly washing buffer and conjugation washing buffer again, and concentrated to ˜0.3 nM by centrifugation at 7000×g for 10 minutes and the pellet was resuspended in conjugation wash buffer. Particles with nucleic acid and TAT-peptide coating were stored at 4° C. prior to adding to cells.

### Particle Transfection and Femtosecond Laser Irradiation

hESC including H1 (passage 38-40), H7 (passage 40-42), H9 (passage 60-70), and H14 (passage 66-68) on 6-well plates were dissociated by rinsing and incubating with 500 μL PBS (Ca²⁺ and Mg²⁺ free, Invitrogen, Life Technologies, Thermo Fisher Scientific, Inc., Waltham, Mass.) at 37° C. in 5% CO₂ atmosphere for 10 minutes followed by manual dissection to suspend the cells. The suspended cell aggregates solution was added to 1 mL mTeSR1+10% FBS and pipetted gently with a P1000 pipette for 10-15 times to further decrease the size of the cell aggregates. Thereafter, cells in the format of single cell or small cell aggregates (5-10 cells per aggregate) were centrifuged at 145×g and resuspended in mTeSR1+10% FBS at ˜2×10⁶ cells/mL. 13 pM of coated particles (after brief sonication) were added to 200 μL of cell suspension and incubated in 1.5 mL Eppendorf tubes at 37° C. for two hours, with gentle pipetting of the solution by P1000 pipette for five times every 30 minutes. Cells were washed by adding 1.2 mL cold PBS, centrifuging at 55×g for three minutes and resuspending in 45 μL cold PBS. Tubes with ˜50 μL of cell suspension were irradiated with 2.4 W/cm² pulsed NIR laser for 15 seconds by the output of a femtosecond Ti:sapphire regenerative amplifier (SPITFIRE, Newport Inc., Irvine, Calif.) with the same setup as previously described (Braun et al., 2009, ACS Nano 3:2007-2015; Huang et al., 2014, Nano Lett. 14(4):2046-2051). The laser beam diameter was ˜4 mm with the spectral range of 800±6 nm, and the pulse duration was ˜130 fs with the repetition rate at 1 kHz. The cells were either used for fluorescence intensity measurements by flow cytometry or plated in MATRIGEL-coated 12-well plates (˜2×10⁵ cells per well) in the presence of 10 μM ROCK inhibitor over the first 24 hours.

### Imaging of Particles in Cells

hESC were dissociated into single cells and small aggregates by PBS treatment as described above followed by seeding on a MATRIGEL-coated 4-well chamber PERMANOX slide (LAB-TEK, Thermo Fisher Scientific, Inc., Waltham, Mass.) (˜4×10⁴ cells per well) in the presence of 10 μM ROCK inhibitor. The next day, coated nanoparticles were added at 13 pM after the medium switch to mTeSR1+10% FBS, and cells were incubated at 37° C. in 5% CO₂ atmosphere for two hours. After that, cells were washed in PBS twice, fixed in 4% paraformaldehyde (PFA) (VWR International, LLC, Radnor, Pa.) in PBS, and washed in PBS again. Cell nuclei were stained using Hoechst 33342 (Sigma-Aldrich, St. Louis, NO). Samples were mounted with Gel/Mount (Electron Microscopy Sciences, Hatfield, Pa.) and visualized under light and fluorescence microscopy. Dark-field scattering images were recorded using an upright microscope (BX51, Olympus America Inc., Center Valley, Pa.) with a reflection-mode high numerical aperture darkfield condenser (U-DCW, 1.2-1.4, Olympus America Inc., Center Valley, Pa.). A 100×/1.30 oil Iris objective (UPLANFL, Olympus America Inc., Center Valley, Pa.) was used to collect only the scattered light from the samples. Images were recorded using a QImaging Retiga-2000R Fast 1394 camera (Qimaging, Surrey, BC, Canada) with RGB color filter module, while fluorescence images were taken under the mono module. Laser scanning confocal microscopy was performed using a FLUOVIEW 1000 (Olympus America Inc., Center Valley, Pa.), with 405 nm and 559 nm lasers under the presets of DAPI (blue) and Quasar570 (red), in sequential linescan mode. 24 slices in Z-stack with 0.4 μm increments were obtained from a single cell scan, and images were then digitally assembled using Imaris software to generate the 3D reconstruction image of cells.

### Flow Cytometry Analysis

Fluorescence intensity of GFP expression and the immunocytochemistry staining levels of OCT4, SSEA and TRA-1-60 were measured using a flow cytometer (ACCURI C6, BD Biosciences, San Jose, Calif.) with a flow rate of 14 μL/min. Quantification of the particle internalization was achieved through flow cytometry fluorescence measurement of Quasar570. The gate was based on the lineage range of forward and side scattering plots, and 10,000 gated events were collected for each sample. To assay particle internalization from Quasar570 and GFP expression, cells were dissociated into single cells with ACCUTASE cell detachment solution (Life Technologies, Thermo Fisher Scientific, Inc., Waltham, Mass.) and collected to inject into the flow cytometer for analysis. OCT4, SSEA4 and TRA-1-60 protein quantification was performed by collecting cells in suspension and staining through immunocytochemistry as follows: cells were fixed in 4% PFA in PBS (4° C., 20 minutes) after the dissociation of cells using ACCUTASE. For OCT4 immunocytochemical staining, additional cell permeabilization was performed in 0.2% Triton-X-100 (Sigma-Aldrich, St. Louis, Mo.) with 0.1% bovine serum albumin (BSA) (Sigma-Aldrich, St. Louis, Mo.) for three minutes. After washing in PBS with 0.5% BSA, approximately 200 μL of cell suspension containing 2×10⁵ cells were incubated with primary antibodies OCT4 (#sc-5279, Santa Cruz Biotechnology, Inc., Dallas, Tex.), SSEA4 (#MAB4304, EMD Millipore, Billerica, Mass.), and TRA-1-60 (#MAB4360, EMD Millipore, Billerica, Mass.) for 30 minutes at room temperature. Cells were then collected by centrifugation, washed in 0.5% BSA and incubated with secondary antibody Alexa Fluor 488 Goat-anti-Mouse IgG, IgM (H+L) (Invitrogen A10680, Life Technologies, Thermo Fisher Scientific, Inc., Waltham, Mass.) for 30 minutes at room temperature. Finally, cells were washed, resuspended in 200 μL PBS, and injected in the flow cytometer for analysis.

### Immunocytochemistry of Attached Cells

Attached cells in culture were washed with PBS, fixed with 4% PFA in PBS (4° C., 20 minutes) and washed with PBS again. Cells were then permeabilized by incubating with the blocking solution [1% Goat Serum (Invitrogen, Life Technologies, Thermo Fisher Scientific, Inc., Waltham, Mass.)+1% BSA (Sigma-Aldrich, St. Louis, Mo.)+0.1% NP40 (Sigma-Aldrich St. Louis, Mo.)] for one hour at room temperature. Thereafter, cells were incubated with primary antibodies OCT4, (#T8660, Sigma-Aldrich, St. Louis, Mo.), α-smooth muscle actin (#A5228, Sigma-Aldrich, St. Louis, Mo.) and α-fetoprotein (#sc-166325, Santa Cruz Biotechnology, Inc., Dallas, Tex.) diluted in the blocking solution for one hour at room temperature. After three PBS washes, all samples were incubated with secondary antibody Alexa Fluor 488 Goat-anti-Mouse IgG, IgM (H+L) diluted in the blocking solution for one hour at room temperature. Following the nuclei staining using Hoechst 33342 (Sigma-Aldrich, St. Louis, Mo.), cells were imaged with an inverted microscope (IX70, Olympus America Inc., Center Valley, Pa.).

### Cell Viability Assay

The effect of particle internalization and NIR laser treatment to stem cell viability was assayed by cell coverage in wells after culturing the treated cells in plates. The treated or untreated control cells were seeded on MATRIGEL-coated 12-well plate at 2×10⁵ cells per well and cultured using mTeSR1 medium for five days (in the presence of 10 μM ROCK inhibitor in the first 24 hours). After washing with cold PBS twice, cells were stained with crystal violet (Sigma-Aldrich, St. Louis, Mo.) solution (1% in PBS) for 10 minutes, followed an additional four washes with PBS. Only the area covered by cells was stained. The wells were imaged by a digital camera and the stained areas in the images were analyzed using ImageJ software (Schneider et al., 2012, Nature methods 9(7):671-675).

### Western Blotting

Cells in 6-well plate were lysed with RIPA lysis buffer (50 mM Tris, 150 mM NaCl, 1% Triton, 0.5% deoxycholate, and 2 mM EDTA) supplemented with Complete Protease Inhibitor Cocktail (Promega Corp., Madison, Wis.) and harvested by manual dissection to dislodge the cells from the plate. The cell extract was collected in 1.5 mL Eppendorf tube and incubated on ice for 30 minutes, followed by centrifugation at approximately 12400×g, 4° C. for 20 minutes. Loading buffer (6×, 300 mM Tris-HCl, 0.01% w/v bromophenol blue, 15% v/v glycerol, 6% w/v SDS and 1% v/v beta-mercaptoethanol) was added to the supernatant and boiled at 95° C. for 10 minutes. Samples were then run on a precast 10% SDS-PAGE gel (Bio-Rad Laboratories, Inc., Hercules, Calif.). Proteins on the gel were electro-transferred onto a nitrocellulose membrane (Bio-Rad Laboratories, Inc., Hercules, Calif.). The membrane was blocked with 5% BSA in PBST at room temperature for 30 minutes followed by 4° C. overnight incubation with primary antibodies OCT4 (#sc-5279, Santa Cruz Biotechnology Inc., Dallas, Tex.) and (β-actin (#ab8227, Abcam plc, Cambridge, UK) diluted in 5% BSA-PBST buffer. The membrane was subsequently washed three times for 15 minutes using PBST and then incubated for three hours at room temperature in 5% BSA-PBST with secondary antibodies including Alexa Fluor 488 labeled goat anti-mouse IgG (#A11001, Invitrogen, Life Technologies, Thermo Fisher Scientific, Inc., Waltham, Mass.) and Alexa Fluor 647 labeled goat anti-rabbit IgG (#A21244, Invitrogen, Life Technologies, Thermo Fisher Scientific, Inc., Waltham, Mass.), followed by washing three times for 15 minutes using PBST. Images were acquired using a scanner system (TYPHOON 9400, GE Healthcare Bio-Sciences, Pittsburgh, Pa.) and the bands were analyzed using the scanner control software.

### Reverse Transcription PCR

Total cell RNA was extracted using RNeasy Mini Kit (Qiagen, Inc., Valencia, Calif.) according to the manufacturer's instructions, and RNase-Free DNase Set (Qiagen, Inc., Valencia, Calif.) was used to remove genomic DNA contamination during the extraction. ˜1 μg total RNA from each sample was subjected to reverse transcription (RT) reaction using High Capacity RNA-to-cDNA kit (Life Technologies, Thermo Fisher Scientific, Waltham, Mass.) following the manufacturer's instructions. Final RT products were diluted 10-fold in water and PCR reactions were performed using GoTag Flexi DNA Polymerase (Promega Corp., Madison, Wis.). 1 μL of cDNA was added in a total volume of 25 μL containing 2 mM MgCl₂, 1×PCR buffer, 0.2 mM deoxyribonucleotide triphosphates (dNTPs), 0.5 μM each of the primers (Table 4, Integrated DNA Technology, Coralville, Iowa) and 0.625 unit of Taq DNA polymerase. The PCR settings for all the genes were as follows: 95° C. for two minutes, 30 cycles through 95° C. for 30 seconds, 59° C. for 30 seconds, and 72° C. for 30 seconds, then extension at 72° C. for five minutes. Reaction for housekeeping gene GAPDH was run as control. 2 μL of the PCR mixture was electrophoresed on 8% Native-PAGE gel at 300 V for 1.5 hours, followed by CYBER Gold (Life Technologies, Thermo Fisher Scientific, Waltham, Mass.) staining and imaging with a scanner system (TYPHOON 9400, GE Healthcare Bio-Sciences, Pittsburgh, Pa.).

TABLE 4
Primers for RT-PCR analysis

| Primers for RT-PCR | | Sequence (5′→3′) | Germ layer |
|---|---|---|---|
| TUBB3 | Forward | GCGGATCAGCGTCTACTACAACGAG | Ectoderm |
| | Reverse | TCAGGCCTGAAGAGATGTCCAAAGG | |
| MAP2 | Forward | GTGGCGGACGTGTGAAAATTGAGAG | |
| | Reverse | ACGCTGGATCTGCCTGGGGACTGTG | |
| PAX6 | Forward | CATTATCCAGATGTGTTTGCCCGAG | |
| | Reverse | TGGTGAAGCTGGGCATAGGCGGCAG | |
| MSX1 | Forward | CGAGAGGACCCCGTGGATGCAGAG | Mesoderm |
| | Reverse | GGCGGCCATCTTCAGCTTCTCCAG | |
| BRACHYURY | Forward | CCCTCTCCCTCCCCTCCACGCACAG | |
| | Reverse | GGCGCCGTTGCTCACAGACCACAGG | |
| FOXA2 | Forward | TGGGAGCGGTGAAGATGGAAGGGCAC | Endoderm |
| | Reverse | TCATGCCAGCGCCCACGTACGACGAC | |
| SOX17 | Forward | CGCTTTCATGGTGTGGGCTAAGGACG | |
| | Reverse | TAGTTGGGGTGGTCCTGCATGTGCTG | |
| AFP | Forward | GAATGCTGCAAACTGACCACGCTGGAAC | |
| | Reverse | TGGCATTCAAGAGGGTTTTCAGTCTGGA | |
| CK8 | Forward | CCTGGAAGGGCTGACCGACGAGATCAA | |
| | Reverse | CTTCCCAGCCAGGCTCTGCAGCTCC | |
| CK18 | Forward | AGCTCAACGGGATCCTGCTGCACCTTG | |
| | Reverse | CACTATCCGGCGGGTGGTGGTCTTTTG | |
| CDX2 | Forward | GCAGAGCAAAGGAGAGGAAA | |
| | Reverse | CAGGGACAGAGCCAGACACT | |
| GAPDH | Forward | GTGGACCTGACCTGCCGTCT | House-keeping |
| | Reverse | GGAGGAGTGGGTGTCGCTGT | |

### Statistical Analysis

Data with error bars are from at least 3 replicate experiments (except for FIG. 28a from duplicate experiments), and are presented as the mean±standard deviation (SD). Statistical analyses were done using the statistical package Instat (GraphPad Software, Inc., La Jolla, Calif.). The means of triplicate samples were evaluated using t-test or one-way ANOVA as indicated in the figure legends.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-6. (canceled)

7. A method comprising:
administering to a subject a composition comprising:
a substrate comprising a surface;
a cleavable linker affixed to at least a portion of the surface; and
a cargo molecule bound to the cleavable linker; and
cleaving the cleavable linker, thereby releasing the cargo molecule from the substrate.

8. The method of claim 7 wherein cleaving the cleavable linker comprises exposing the cleavable linker to near infrared radiation.

9. The method of claim 7 wherein the cargo molecule comprises a polypeptide.

10. The method of claim 7 wherein the cargo molecule comprises a polynucleotide.

11. A method comprising:
introducing into a cell a composition comprising:
a substrate comprising a surface;
a cleavable linker affixed to at least a portion of the surface; and
a cargo molecule bound to the cleavable linker; and
cleaving the cleavable linker, thereby releasing the cargo molecule from the substrate.

12. The method of claim 11 wherein cleaving the cleavable linker comprises exposing the cleavable linker to near infrared radiation.

13. The method of claim 11 wherein the cargo molecule comprises a polypeptide.

14. The method of claim 11 wherein the cargo molecule comprises a polynucleotide.

15. The method of claim 11 wherein the cell comprises a tumor cell.

16. The method of claim 11 wherein the cell comprises a stem cell.


SEQUENCE LISTING (24 sequences)

SEQ ID NO: 1 - protein, 7 aa, Artificial
  cell targeting peptide
RPARPAR

SEQ ID NO: 2 - protein, 6 aa, Artificial
  transcription peptide
  MISC_FEATURE (2)..(2): X at position 2 is Y or F
  MISC_FEATURE (6)..(6): X at position 6 is K or R
LXPWMX

SEQ ID NO: 3 - protein, 6 aa, Artificial
  transcription peptide
TLKIVW

SEQ ID NO: 4 - protein, 11 aa, Artificial
  human immunodeficiency virus 1
YGRKKRRQRRR

SEQ ID NO: 5 - DNA, 25 bp, Artificial
  anchor oligonucleotide
gccaccacgtctacttgaagtccca

SEQ ID NO: 6 - RNA, 21 nt, Artificial
  anchor oligonucleotide
gggcggcuuugccaagugcuu

SEQ ID NO: 7 - RNA, 23 nt, Artificial
  siRNA
aagcacuuggcaaagccgcccuu

SEQ ID NO: 8 - RNA, 21 nt, Artificial
  siRNA
gggcggcuuugccaagugcuu

SEQ ID NO: 9 - RNA, 23 nt, Artificial
  siRNA
aagcacuuggcaaagccgcccuu

SEQ ID NO: 10 - RNA, 50 nt, Artificial
  oligonucleotide
ugggacuucaaguagacgugguggcuuaagcacuuggcaaagccgcccuu

SEQ ID NO: 11 - RNA, 50 nt, Artificial
  oligonucleotide
ugggacuucaaguagacgugguggcuuaagggcggcuuugccaaguguuu

SEQ ID NO: 12 - RNA, 21 nt, Artificial
  oligonucleotide
gggcggcuuugccaagugcuu

SEQ ID NO: 13 - RNA, 21 nt, Artificial
  oligonucleotide
gcacuuggcaaagccgcccuu

SEQ ID NO: 14 - DNA, 25 bp, Artificial
  oligonucleotide
accctgaagttcatctgcaccaccg

SEQ ID NO: 15 - DNA, 25 bp, Artificial
  oligonucleotide
cggtggtgcagatgaacttcagggt

SEQ ID NO: 16 - DNA, 25 bp, Artificial
  oligonucleotide
accctgaagttcatctgcaccaccg

SEQ ID NO: 17 - DNA, 25 bp, Artificial
  oligonucleotide
gccaccacgtctacttgaactccca

SEQ ID NO: 18 - RNA, 23 nt, Artificial
  oligonucleotide
gcuuguucguugguaacuacauu

SEQ ID NO: 19 - RNA, 25 nt, Artificial
  siRNA
acccugaaguucaucugcaccaccg

SEQ ID NO: 20 - RNA, 27 nt, Artificial
  siRNA
cgguggugcagaugaacuucaggguca

SEQ ID NO: 21 - RNA, 25 nt, Artificial
  siRNA
ggaugugguccgagugugugguucg

SEQ ID NO: 22 - RNA, 25 nt, Artificial
  siRNA
uuaaccacacucggaccacauccuu

SEQ ID NO: 23 - DNA, 25 bp, Artificial
  oligonucleotide
accctgaagttcatctgcaccaccg

SEQ ID NO: 24 - DNA, 25 bp, Artificial
  oligonucleotide
cggtggtgcagatgaacttcagggt